(19) United States
(12) Patent Application Publication
(10) Pub. No.: US 2020/0262916 A1
(43) Pub. Date: Aug. 20, 2020

(54) Mammalian Atg8 Proteins Control Autolysosomal Biogenesis Through SNARES

(71) Applicant: STC. UNM, ALBUQUERQUE, NM (US)

(72) Inventors: Vojo P. Deretic, Placitas, NM (US); Yuexi Gu, Albuquerque, NM (US); Graham Timmins, Albuquerque, NM (US)

(21) Appl. No.: 16/791,520

(22) Filed: Feb. 14, 2020

Related U.S. Application Data
(60) Provisional application No. 62/806,004, filed on Feb. 15, 2019.

(51) Int. Cl. C07K 16/28; A61K 31/4439; A61K 31/5025; A61K 31/502; A61K 31/53

(52) U.S. Cl. CPC C07K 16/28; A61K 31/4439; A61K 45/06; A61K 31/502; A61K 31/53; A61K 31/5025

(57) ABSTRACT

The present invention is directed to the elucidation of the mechanism that human Atg8 protein regulates endolysosomal systems in the cell and the role this protein plays in mediating autophagy. Methods of treating autophagy-mediated disease states with agonists/antagonists of Atg8, STX16 and STX17 proteins are disclosed as are pharmaceutical compositions.

### RELATED APPLICATIONS

This application claims the benefit of priority of priority of U.S. provisional application No. 62/806,004, filed Feb. 15, 2019 of identical title, the entire contents of which application is incorporated by reference in this application.

### GOVERNMENT SUPPORT

This invention was made with government support under R37AI042999, R0IA142999 and P20GM121176 awarded by the National Institutes of Health (NIH). The government has certain rights in the invention.

### FIELD OF THE INVENTION

The present invention is directed to the elucidation of the mechanism that human Atg8 protein regulates endolysosomal systems in the cell and the role this protein plays in mediating autophagy. Additional embodiments are directed to making use of these findings to provide therapies, including the use of ATg8, STX16 and/or STX17 modulators (inhibitors or agonists) for the treatment of autophagy mediated disease states and/or conditions.

### BACKGROUND AND OVERVIEW OF THE INVENTION

The autophagy pathway, controlled by a conserved set of ATG genes (Levine & Kroemer, 2019, Mizushima et al., 2011), is a cytoplasmic homeostatic process at an interface between quality control and metabolism. This pathway can be activated by metabolic and stress inputs (Garcia & Shaw, 2017, Marino et al., 2014, Noda & Ohsumi, 1998, Saxton & Sabatini, 2017, Scott et al., 2004) or triggered by cargo recognition leading to in situ assembly of ATG factors via receptor-regulators (Kimura et al., 2016, Lazarou et al., 2015, Mandell et al., 2014). Autophagy can be bulk or selective (Birgisdottir et al., 2013, Randow & Youle, 2014, Stolz et al., 2014), whereas the multiple biological outputs of autophagy depend on its completion and the type of termination of the autophagic pathway, e.g. degradation or secretion (Levine & Kroemer, 2019, Ponpuak et al., 2015). Bulk autophagy, whereby portions of the cytoplasm of heterogeneous composition are sequestered, occurs during starvation, and can be quantified by imaging (An & Harper, 2018) or biochemically (Engedal & Seglen, 2016, Seglen et al., 2015) by following sequestration of the cytosolic enzyme LDH, or ultrastructurally by enumerating partially degraded electron-dense ribosomes (Eskelinen, 2008, Tanaka et al., 2000). Autophagy can be selectively guided to specific cargo via a number of sequestosome l/p62-like receptors (SLRs) (Birgisdottir et al., 2013, Bjorkoy et al., 2005, Stolz et al., 2014) or other classes of receptors besides SLRs (Kimura et al., 2016, Levine & Kroemer, 2019). Autophagy machinery is often recruited to damaged or dysfunctional targets after they are marked for autophagic degradation by ubiquitin and galectin tags that in turn are recognized by cytosolic autophagic receptors such as SLRs (Randow & Youle, 2014, Stolz et al., 2014). Autophagy apparatus can also be directly recruited via receptors resident to organelles if they become exposed following organellar membrane damage or depolarization, or are modified downstream of physiological or developmental signals (Bhujabal et al., 2017, Liu et al., 2012, Sandoval et al., 2008, Wei et al., 2017, Wild et al., 2011).

Degradative autophagy terminates in fusion of autophagosomes with lysosomes whereby the sequestered cargo is degraded. Examples of selective degradative autophagy include autophagy of mitochondria (mitophagy), peroxisomes (pexophagy), intracellular microbes (xenophagy), ribosomes (ribophagy), protein aggregates (aggrephagy), specific intracellular multiprotein complexes (precision autophagy) (An & Harper, 2018, Birgisdottir et al., 2013, Kimura et al., 2016, Randow & Youle, 2014). Given the diversity of cargo, complexity of the protein components and membrane compartments engaged, as well as the exquisite responsiveness to a variety of cargo-triggers (e.g. damaged organelles, aggregates) and stress conditions (e.g. starvation, hypoxia), autophagy is controlled by a collection of subsystems that have to come together in a modular fashion and cooperate in the initiation and execution of autophagy (Mizushima et al., 2011). These include: (i) formation of a complex between the ULK1 kinase, FIP200, ATG13 and ATG101, transducing mTOR inhibition (Ganley et al., 2009, Hosokawa et al., 2009, Jung et al., 2009) and AMPK activation (Kim et al., 2011) to induce autophagy; (ii) generation of PDP by the ATG14-endowed Class in PI3-Kinase Complex VPS34 (Baskaran et al., 2014, Chang et al., 2019, Petiot et al., 2000) that includes Beclin 1 (He & Levine, 2010), which can also be modified by AMPK to specifically activate the ATG14 form of VPS34 (Kim et al., 2013); (iii) the ubiquitin-like conjugation system with ATG5-ATG12/ATG16L1 (Mizushima et al., 1998a, Mizushima et al., 1998b) acting as an E3 ligase to lipidate mammalian homologs of yeast Atg8 (mAtg8s), some of which like LC3B have become key markers for autophagosomal membranes (Kabeya et al., 2000); (iv) the only integral membrane ATG protein, ATG9, and the ATG2-WIPI protein complexes, of still unknown but essential functions (Bakula et al., 2017, Velikkakath et al., 2012, Young et al., 2006). These modules are for the most part interconnected, with FIP200 physically bridging via ATG16L1 the ULK1/2 complex with the mAtg8s conjugation system (Fujita et al, 2013, Gammoh et al., 2013, Nishimura et al., 2013), ATG16L1 and WIPI directly interacting (Dooley et al., 2014), ATG13 connecting the ULK1/2 complex with VPS34 via ATG13's HORMA domain binding to ATG14 of the VPS34 complex (Jao et al., 2013, Park et al., 2016), and PI3P, the product of VPS34 (Petiot et al., 2000), being detected on membranes by WIPIs (Bakula et al., 2017).

hi yeast, a component of the above systems morphologically equated with autophagy is Atg8, with its appearance as a single punctum defining the pre-autophagosomal structure (Kirisako et al., 1999). In mammals, this function is spread over a set of six mAtg8s (Mizushima et al., 2011, Weidberg et al., 2010), LC3A,B,C, GABARAP, -L1, and -L2. Although it is generally accepted that mAtg8s are important for autophagy, and that mAtg8 lipidation and puncta formation herald autophagy induction and isolation membrane formation, tire core function of mAtg8s and possibly a direct role in autophagosomal membrane remodeling remains to be defined. Several models, including a direct role of Atg8/mAtg8s in catalyzing membrane fusion (Nakatogawa et al., 2007, Weidberg et al., 2011, Weidberg et al., 2010) have been considered but later challenged (Nair et al., 2011). Nevertheless, many of the cargos, selective autophagy receptors, and regulatory components (ULK, ATG13, FIP200, ATG14, Beclin 1, VPS34, ATG4) contain LC3-interacting regions (LIRs) and can either recruit cargo (Birgisdottir et al., 2013, Bjorkoy et al., 2005) or organize core autophagy machinery (Alemu et al., 2012, Birgisdottir et al., 2019, Skytte Rasmussen et al., 2017). Furthermore, recent studies show that even in the complete absence of mAtg8s, autophagosome formation proceeds, albeit with a lower sequestration volume, and that mAtg8s are important for autolysosomal formation (Nguyen et al., 2016a). In keeping with the latter findings, depletion of components participating in mAtg8 conjugation delays autophagic flux progression (Tsuboyama et al., 2016). It remains to be understood precisely how mAtg8s control membrane trafficking and remodeling during phagophore formation and elongation (Xie et al., 2008) and during autophagosome-lysosome fusion (Nguyen et al., 2016a, Weidberg et al., 2010).

Degradative aspects of autophagy depend on autophagosomes fusion with organelles of the endolysosomal system, often referred to as autophagic flux or autophagosome-lysosome fusion (Klionsky et al., 2016, Tanaka et al., 2000). Much of the intracellular membrane trafficking and fusion processes are catalyzed by SNARE proteins, which ensure compartment/organelle specificity through pairing of cognate Qa-, Qb-, Qc- and R-SNARE partners (Jahn & Scheller, 2006). SNAREs are often found in compartments where they function but also transit through other membranes, and thus their fusion activities are tightly regulated by tethers, SM (Sec1/Munc18) proteins, Rabs, and additional factors that modulate their recruitment, activation, cycles of reuse, etc., (Hong & Lev, 2014). In the context of autophagy, SNAREs have been studied at different stages along the autophagy pathway (Itakura et al., 2012, Kimura et al., 2017, Moreau et al., 2013, Nair et al, 2011). At the autophagosome-lysosome fusion stage, an initially identified SNARE was the Qa-SNARE Stx17, forming complexes with Qbc-SNARE SNAP29 and R-SNARE VAMP8 (Diao et al., 2015, Guo et al., 2014, Itakura et al., 2012, Takats et al., 2013, Wang et al., 2016). Recent studies have indicated that additional SNAREs may be required or even be dominant in tins process (Matsui et al., 2018, Takats et al., 2018). Thus, redundant, complementary or synergistic SNARE-driven fusion and regulatory events may be at work during auto-lysosomal fusion. We have recently shown that Stx17 interacts via its LIR motif with mAtg8s, which functions in Stx17's recruitment to autophagosomes (Kumar et al., 2018), and that TBK1-phosphorylated Stx17 plays a role in autophagy initiation (Kumar & al., 2019), in keeping with studies by others (Arasaki et al., 2018, Arasaki et al., 2015, Hamasaki et al., 2013). Thus, Stx17 contributes to the autophagy pathway at several stages from initiation to maturation.

A report of direct interaction between mAtg8s and Stx17 (Kumar et al, 2018) has set an unanticipated precedent, and in the present application the inventors explored whether there is a broader range of interactions between mAtg8s and mammalian SNAREs. Using bioinformatics and biochemical approaches, we found additional candidate SNAREs with LIR motifs that bind mAtg8s. Among the candidates, the inventors focused on a cognate set of SNAREs, Stx16, Vti1a and Stx6, previously implicated in retrograde transport between endosomes and the trans-Golgi network (TGN). Through mutational and functional analyses, the inventors unexpectedly found that Stx16 acts synergistically with Stx17 and is important for diverse types of autophagy, including mitophagy, pexophagy, ribophagy, and elimination of intracellular Mycobacterium tuberculosis. The inventors have further found that Stx16 is important for cellular lysosomal content and function, and that mAtg8s modulate Stx16 localization on endolysosomal organelles. This uncovers a mechanism different from the previous views of how mAtg8s help grow autophagosomal and complete autolysosomal membranes. We conclude that mAtg8s control autophagy at least in part by directly binding to SNARE proteins engaged in the biogenesis of the endolysosomal and autolysosomal organelles.

The autophagy pathway terminates in autophagosome fusion with organelles of the endolysosomal pathway to generate autolysosomes where the captured cargo is degraded. Fusion events leading to transformation of autophagosomes into autolysosomes has become a focus of attention; however, a unified understanding of key events is still not fully available. Mammalian homologs of the yeast Atg8 protein (mAtg8s) play a central role in autophagy, but their potential functions in autophagosomal maturation has not been defined.

Our studies on the mechanisms of Stx16 suggest that Stx16 functions in delivery of lysosomal membrane proteins, likely from TGN via late endosomes. Furthermore, mAtg8s proteins control proper localization of Stx16 in late endosome and lysosomes through their interactions mediated by the LIR motif on Stx16. This may partially account for the mechanisms as to how mAtg8s regulate endolysosomal system in the cell.

### BRIEF DESCRIPTION OF THE INVENTION

The present inventors employed peptide arrays and bioinformatics to identify LC3-interacting regions (LIRs) in the SNARE family proteins, which are known to regulate intracellular membrane fusion and trafficking. A Qa SNARE, syntaxin 16 (Stx16), as well as its cognate Qb and Qc SNAREs, Vti1a and Stx6, all bear LIR motifs upstream of their SNARE domains. Using CRISPR/Cas9-mediated knockout, the inventors confirmed that autophagic flux still continues in a panel of STX17-knockout cells, suggesting Stx17 alone is insufficient to drive autophagosome fusion with lysosomes/endosomes. Knockout of both STX16 and STX17 blocked autophagic flux as shown by accumulation of lipidated LC3B, as well as decreased mitophagy. Further analysis of the mechanisms revealed that Stx16 is required for the efficient delivery of lysosomal membrane proteins from the trans-Golgi network; similar effects were observed in cells knocked out for all six mATG8s (hexa KO). These observations suggest that mAtg8s control the Stx16 SNARE complex to regulate autolysosomal biogenesis and endolysosomal homeostasis. Thus, Atg8 or an agonist of Atg8 and/or STX16 and/or STX17 or an agonist thereof may be used to upregulate autophagy and influence disease states and/or conditions in which autophagy mediates the disease slate and/or condition. Likewise, an inhibitor of Atg8 (such as an anti-Atg8 antibody or a short Atg3 peptide) or a related inhibitor as described herein and/or an inhibitor of STX16 and/or STX17 may be used in the treatment of autophagy mediated diseases where autophagy is upregulated and would benefit from inhibition.

The inventors' data and other's data (Nguyen et al. 2016) have shown that knockout of STX17 alone does not block autophagic flux completely, suggesting additional SNARE complexes in mediating autophagosomal fusion with lysosomes/endosomes. Here the inventors identify Stx16/Vti1a/Stx6 complex as additional SNAREs that synergize with Stx17 for autophagosomal maturation. Knockout of both STX16 and STX17 effectively blocks autophagic flux dining starvation-induced bulk autophagy, and inhibits autophagic clearance of damaged mitochondria.

Accordingly, the present invention is directed to a method of therapy of an autophagy disease state and/or conditions, the method comprising administering to a patient in need an effective amount of at least one Atg8 modulator (e.g. an agonist or inhibitor) in order to treat an autophagy mediated disease state and/or conditions. Additional embodiments are directed to the use of a Syntaxin 16 (Stx16) modulator, alone or preferably in combination with a Syntaxin 17 (Stx17) modulator in order to treat an autophagy disease state and/or condition. Embodiments of the present invention are directed to the unexpected discovery that modulators of Stx 16 and stx 17, when combined and used in therapy (as combined agonists or inhibitors of Stx 16 and Stx17, respectively) can provide a synergistically favorable therapeutic effect on those autophagy-mediated disease states and/or conditions which respond favorable to inhibition and/or upregulation of autophagy. Thus, in those disease states and/or conditions which are mediated through autophagy and which favorably respond to the inhibition of Stx 16 and Stx 17 (and the inhibition of autophagy), there will accompany a synergistic effect when at least one inhibitor of each of Stx 16 and Stx 17 and optionally Atg8 are combined to treat autophagy disease states and conditions, especially cancer and autoimmune diseases such as rheumatoid arthritis, malaria, antiphospholipid antibody syndrome, lupus, chronic urticarial, Sjogren's disease, autoimmune-related Type 1 diabetes, rheumatoid arthritis (RA), psoriasis/psoriatic arthritis, multiple sclerosis, inflammatory bowel disease (IBD) including Crohn's disease and ulcerative colitis, Addison's disease, Grave's disease, Hashimoto's thyroiditis. Myasthenia gravis, autoimmune vasculitis, pernicious anemia and celiac disease, hi those disease states and conditions which are mediated through autophagy and which favorably respond to the upregulation of Stx 16 and Stx 17 (and the upregulation of autophagy), there will accompany a synergistic effect when at least one modulator (preferably, an agonist) of each of Stx 16 and Stx 17 and optionally Atg8 are combined and administered to a patient to treat autophagy disease states and conditions, especially disease states and conditions associated with mitophagy, pexophagy, ribophagy and xenophagy, among numerous other autophagy-mediated disease states and/or conditions.

In embodiments, the present invention is also directed to pharmaceutical compositions comprising an effective amount of at least one ATg8 modulator optionally in combination with a pharmaceutically acceptable carrier, additive or excipient. Additional pharmaceutical compositions comprise a combination of a STX16 and STX17 inhibitor and optionally, an inhibitor of Atg8 in combination with a pharmaceutically acceptable carrier, additive and/or excipient for the treatment of autophagy mediated disease states and conditions which respond favorably to autophagy inhibition, especially including cancer and autoimmune diseases such as rheumatoid arthritis, malaria, antiphospholipid antibody syndrome, lupus, chronic urticarial, Sjogren's disease, autoimmune-related Type 1 diabetes, rheumatoid arthritis (RA), psoriasis/psoriatic arthritis, multiple sclerosis, inflammatory bowel disease (IBD) including Crohn's disease and ulcerative colitis, Addison's disease, Grave's disease, Hashimoto's thyroiditis, Myasthenia gravis, autoimmune vasculitis, pernicious anemia and celiac disease. These compositions show a synergistic therapeutic effect in treating autophagy-mediated disease states and/or conditions which respond to autophagy inhibition. In other embodiments, the present invention is directed to pharmaceutical compositions comprising an effective amount of a combination of a STX 16 and STX 17 agonist and optionally, an agonist of Atg8 in combination with a pharmaceutically acceptable carrier, additive and/or excipient for the treatment of autophagy mediated disease states and/or conditions which respond favorably to autophagy upregulation including disease states associated with mitophagy, pexophagy, ribophagy and xenophagy, among other disease states and/or conditions as otherwise described and identified herein.

In another embodiment, tire present invention is directed to the use of an effective amount of at least one agonist of STX17 for treating coronavirus infections in an infected patient in need. In this embodiment, a patient who has been diagnosed with coronavirus or who is suspected of being infected with coronavirus is administered an effective amount of at least one STX17 agonist, optionally in combination with an effective amount of at least one STX16 agonist and/or ATg8 agonist as described herein. Pharmaceutical compositions useful for the treatment of coronavirus comprise a therapeutically effective amount of at least one STX17 agonist and optionally at least one STX16 agonist and/or at least one ATg8 agonist in combination with a pharmaceutically acceptable carrier, additive and/or excipient.

Additional embodiments of the present invention may be readily gleaned from a review of the detailed description of the invention, including the examples, which follows.

### BRIEF DESCRIPTION OF THE FIGURES

FIG. 1 shows that many SNAREs bind mammalian Atg8 proteins. (A) shows a peptide array dot blot analysis to identify LIR motifs in the indicated SNARE proteins. Amino acids for the identified LIRs from positive signals are marked on each SNARE. Amino acids are denoted as amino acid single letter codes in the blot. (B) shows the schematics of the functional domains of Stx16/Vti1a/Stx6 cognate SNAREs with the positions of LIR motifs. The LIR motif marked in green in syntaxin 16 indicates established LIR based on following analyses. (C) shows the co-immunoprecipitation (Co-IP) analysis of interactions between FLAG-tagged Stx16/Vti1a/Stx6 and EGFP-tagged LC3B or GABARAP overexpressed in HEK293T cells. * indicates mouse IgG heavy chain of precipitated mouse-anti-FLAG antibody. (D) shows GST pull-down analysis of interactions between radiolabeled Myc-Stx16 and GST-tagge+d mAtg8 proteins. (E) shows the GST pull-down analysis of interactions between wild type (WT) or LIR-mutant (L219A-V222A) Stx16 and GST-tagged LC3C or GABARAP. Lower panel shows percentages of WT or LIR-mutant Stx16 bound to GST-LC3C or GST-GABARAP. Data shown as means±SEM of precipitated Stx16, n=3. (F) shows GST pull-down analysis of interactions between WT or different types of LIR-mutant Stx16 and GS+T-tagged LC3C or GABARAP. Lower panel shows percentages of WT or +LIR-mutant Stx16 bound to GST-LC3C or GST-GABARAP. (F) shows GST pull-down analysis of interactions between radiolabeled Myc-Stx16 and WT or LDS-mutant GABARAP (GABARAP-Y49A and -Y49A/F104A). Lower panel shows percentages of Stx16 bound to WT or LDS-mutant GABARAP. Data shown as means±SEM of precipitated Stx16, n=3.

FIG. 2 shows that mAtg8-interacting SNAREs Stx16 and Stx17 are required for efficient bulk autophagic flux. (A) shows WT or STX17-knockout (STX17^{KO}) HeLa cells were starved with or without the presence of bafilomycin A1 (Baf A1, 100 nM) for 2 h, and cell lysates were subjected to Western blot analysis of LC3B and p62. (B) shows the quantifications of LC3B-II levels normalized to β-actin from cells treated as in (A); Data shown as means±SEM of LC3B-II and β-actin ratios, u=3; *, p<0.05 (one-way ANOVA). (C) shows that WT, STX16^{KO}, STX17^{KO} or STX16/STX17 double KO (STX16/STX17^{DKO}) HeLa cells were starved with or without the presence of Baf A1 (100 nM) for 2 h, and cell lysates were subjected to Western blot analysis of LC3B. (D) shows the Quantifications of LC3B-II levels normalized to β-actin from (C); Data shown as means±SEM of LC3B-II and β-actin ratios, n=3; †, not significant; **, p<0.01 (one-way ANOVA). (E) shows WT or STX16/STX17^{D+KO} HeLa cells were starved in EBSS for 2 h, and subjected to ultrastructural analysis of the autophagic vesicles (AV) with electron microscopy. AVi: initial autophagic vacuoles; AVd: degradative autophagic vacuoles; G: Golgi apparatus. Image acquisition and counting as in Methods. Scale bars, 1 μm and 0.5 μm (top sections). (F) shows quantifications of autophagic vesicles in WT and STX16/STX17^{DKO} HeLa cells treated as (E); Data shown as means±SEM of AV profiles relative to cytoplasmic area; †, not significant; *, p<0.05 (two-way ANOVA). AV profiles from 47 images of each samples were counted.

FIG. 3 shows that Stx16 and Stx17 are required for selective autophagy of mitochondria, peroxisomes, and M. tuberculosis. (A) shows WT or STX16/STX17^{DKO} HeLa-YFP-Parkin cells were treated with CCCP (10 μM) o+r oligomycin A (5 μM) and antimycin A (10 μM) (OA) for 16 hours, and subjected to high-content microscopy (HCM) analysis of mitochondria clearance. Masks: blue, nuclei; red, mitochondria stained with mitochondrial DNA (mtDNA) antibody. Scale bar: 20 μm. (B) shows quantifications of mtDNA by object count or total area per cell in WT or STX16/S+TX17^{DKO} HeLa-YFP-Parkin cells treated as in (A); Data shown as means±SEM of m+tDNA object count (upper panel) or object total area (lower panel) per cell; minimum 500 cells were counted each well from at least 12 wells, 3 independent experiments; **, p<0.01 (two-way ANOVA). (C) shows WT or STX16/STX17^{DKO} Huh7 cells were treated with H₂O₂ (0.4 mM) for indicated time points, and autophagic clearance of peroxisomes was measured by the protein levels of PMP70, PEX14 and p62. (D) shows quantifications of peroxisomal proteins PMP70 and PEX14 for WT or STX16/STX17^{DKO} Huh7 cells treated as in (C); Data shown as means±SEM of PEX14 or PMP70 and β-actin ratios, n=3; †, not significant *, p<0.05; **, p<0.01 (two-way ANOVA). (E) shows WT, STX16^{KO}, STX17^{KO} or STX16/STX17^{DKO} THP-1 cells were infected with Mycobacterium tuberculosis (Mtb), followed by starvation to induce autophagy. Autophagic clearance of infected Mtb was measured by Mtb colonies grown on Middlebrook 7H11 agar plates; Data shown as means±SEM of Mtb colonies, n=4; *, p<0.05; **, p<0.01 (two-way ANOVA). (F) shows schematics for the requirement of Stx16 and Stx17 in autophagic clearance of damaged mitochondria, surplus peroxisomes and invaded bacterial pathogens.

FIG. 4 shows that Stx16 and Stx17 cooperate in ribophagy. (A) shows a schematic of ribophagy detection with Keima-based reporter system; FP: fluorescent protein. (B) shows the validation of STX16/STX17 DKO by Western blot analysis in HEK293 cells stably expressing RPS3-Keima (left panel) and HCT116 cells stably expressing RPL28-Keima (right panel). (C) shows WT or STX16/STX17^{DKO} HEK293 RPS3-Keima cells were cultured in full medium or starved for 8 hours, and subjected to HCM analysis of Keima puncta accumulated in autolysosomes with the excitation wavelength at 560 nm. Masks: white, cells identified based on nuclei; red, Keima puncta detected with the excitation and emission wavelengths of 560 nm and 620 nm, respectively; lower panel shows Keima puncta without masks. Scale bar: 20 μm. (D) shows quantifications of autolysosomal Keima puncta in WT or STX16/STX17^{DKO} HEK293 RPS3-Keima cells treated as in (C). Data shown as means±SEM of Keima puncta per cell, minimum 1000 cells were counted each well from at least 12 wells, 3 independent experiments; **, p<0.01 (two-way ANOVA). (E) shows WT or STX16/STX17^{DKO} HCT116 RPL28-Keima cells were cultured in full medium or starved for 8 hours, and subjected to HCM analysis of Keima puncta accumulated in autolysosomes with the excitation wavelength at 560 nm. Masks: white, cells identified based on nuclei; red, Keima puncta detected with the excitation and emission wavelengths of 560 nm and 620 nm, respectively; lower panel shows Keima puncta without masks. Scale bar: 20 μm. (F) shows quantifications of autolysosomal Keima puncta in WT or STX16/STX17^{DKO} HCT116 RPL28-Keima cells treated as in (E). Data shown as means±SEM of Keima puncta per cell, minimum 1000 cells were counted each well from at least 12 wells, 3 independent experiments; †, not significant; **, p<0.01 (two-way ANOVA).

FIG. 5 shows that Stx16 is required for the maintenance of lysosomal homeostasis and mTOR activity. (A) shows WT or STX16^{KO} HeLa cells were starved in HBSS for 2 h and subjected to HCM analysis of LAMP2 and LC3 puncta. Masks: white, cells identified based on nuclei; red, LC3 puncta; green, LAMP2 puncta: yellow, overlap of LC3 and LAMP2. Scale bar: 20 μm. (B-D) show the quantifications of LC3 puncta per cell (B), LC3 and LAMP2 overlap area per cell (C), and LAMP2 puncta per cell (D) in WT or STX16^{KO} cells treated as in (A). Data shown as means±SEM of puncta or overlap area per cell, minimum 500 cells were counted each well from at least 12 wells, 3 independent experiments; *, p<0.05; **, p<0.01 (two-way ANOVA). (E) shows western blot analysis of LAMP1 and LAMP2 protein levels in WT and separate clones of STX16^{KO} HeLa, Huh7, and U2OS cells. (F) shows western blot analysis of LAMP2 protein levels in WT and separate clones of STX6^{KO} or VT11A^{KO} HeLa cells. (G) Time course of mTOR inactivation during EBSS starvation in WT vs. STX16^{KO} HeLa cells analyzed by Western blotting for mTOR substrates ULK1 and 4E-BP1. (H) shows WT or STX16^{KO} HeLa cells were starved in EBSS for 1 h and subjected to HCM analysis of mTOR puncta. Masks: white, cells identified based on nuclei; red, mTOR puncta. Scale bar: 20 μm. (I) shows quantifications of mTOR puncta in WT or STX16^{KO} cells treated as in (H). Data shown as means±SEM of mTOR puncta per cell, minimum 500 cells were counted each well from at least 12 wells, 3 independent experiments; **, p<0.01 (two-way ANOVA).

FIG. 6 shows that Stx16 regulates proper distribution of acidified compartments in the cell. (A) shows that WT or STX16^{KO} HeLa cells were starved in EBSS for 1 h, followed by starvation with the presence of LysoTracker Red DND-99 (LTR) (100 nM) for additional 30 min, and subjected to HCM analysis of LTR. Scale bar: 20 μm. (B) shows quantifications of LTR count per cell or total area per cell in WT and STX16^{KO} HeLa cells treated as in (A). Data shown as means±SEM of LTR puncta per cell or object total area per cell, minimum 500 cells were counted each well from at least 12 wells, 3 independent experiments; †, not significant (two-way ANOVA). (C) shows that WT or STX16^{KO} HeLa cells were starved in EBSS for 1 h, followed by starvation with the presence of LTR (100 nM) for additional 30 min, and subjected to HCM analysis of the colocalization between LTR and TGN46. Masks: white, cells identified based on nuclei; red, LTR puncta; green, TGN46 puncta; yellow, overlap between LTR and TGN46. Scale bar: 20 μm. (D) shows quantifications of overlaps between LysoTracker Red and TGN46 in WT and STX16^{KO} cells treated as in (C). Data shown as means±SEM of LTR and TGN46 overlap area per cell, minimum 500 cells were counted each well from at least 12 wells, 3 independent experiments; **, p<0.01 (two-way ANOVA). (E) shows WT or STX16/STX17^{DKO} HeLa cells were starved in EBSS for 2 h, and subjected to ultrastructural analysis of the Golgi apparatus with election microscopy. VF of Golgi: volume fraction of Golgi relative to total cytoplasmic area. Scale bar, 2 μm. Red arrows indicate Golgi apparatus.

FIG. 7 shows that mammalian Atg8s regulate Stx16 localization and acidification of endolysosomal organelles. (A) shows WT, LC3^{TKO}, GABARAP^{TKO} or Hexa^{KO} HeLa cells were starved in EBSS for 90 min and subjected to HCM analysis of overlaps between LAMP2 and Stx16. Masks: white, cells identified based on nuclei; green, LAMP2 puncta; red, Stx16 puncta; yellow, overlap of LAMP2 and Stx16. Scale bar: 20 μm. (B) shows quantifications of overlaps between LAMP2 and Stx16 in WT, LC3^{TKO}, GABARAP^{TKO} or Hexa^{KO} HeLa cells treated as in (C). Data shown as means±SEM of LAMP2 and Stx16 overlap area per cell, minimum 500 cells were counted each well from at least 12 wells, 3 independent experiments; *, p<0.05; **, p<0.01 (one-way ANOVA). (C) shows quantifications of overlaps between LBPA and Stx16 in WT, LC3^{TKO}, GABARAP^{TKO} or Hexa^{KO} HeLa cells grown in frill medium or treated as in (A). Data shown as means±SEM of LBPA and Stx16 overlap area per cell, minimum 500 cells were counted each well from at least 12 wells, 3 independent experiments; *, p<0.05; **, p<0.01 (one-way ANOVA). Representative images are shown in Appendix Fig. S5A. (D) shows WT, LC3^{TKO}, GABARAP^{TKO} or Hexa^{TKO} HeLa cells were starved in EBSS for 1 hour, followed by starvation with the presence of LTR (100 nM) for additional 30 min, and subjected to HCM analysis of LTR. Scale bar: 20 pin. (E) shows quantifications of LTR puncta in WT, LC3^{KO}, GABARAP^{TKO} or Hexa^{KO} HeLa cells treated as in (D). Data shown as means±SEM of LTR puncta per cell, minimum 500 cells were counted each well from at least 12 wells, 3 independent experiments; †, not significant; *, p<0.05; **, p<0.01 (one-way ANOVA). (F) shows a schematic model of this study. Mammalian Atg8 proteins directly bind Stx16 through its LIR motif, which controls the proper localization of the Stx16 SNARE complex at the endolysosomal compartments. The Stx16 SNARE complex functions in the maintenance of lysosomal homeostasis upon autophagy induction, thus controlling mTOR activity.

FIGURE EV1 shows peptide arrays identify a set of SNAREs bearing LIR motifs. (A) shows schematics of the functional domains of SNAREs bearing LIR motifs identified through bioinformatic and peptide array analyses. LIR motifs with the amino acid sequences are marked as red bars in approximate positions on each SNARE protein. The LIR motifs in Stx16 and Stx17 are marked green as they are validated LIRs through biochemical and functional analyses. (B) shows endogenous Co-IP analysis of the interactions between LC3 and Stx16, Vti1a in HeLa and U2OS cells. * indicates IgG heavy chain of precipitated anti-LC3 antibody.

FIGURE EV2 shows that Stx16 and Stx17 are required for efficient autophagic flux induced by starvation. (A) shows sequences of guide RNAs (gRNA) targeting STX17 and STX16. Protospacer adjacent motif (PAM) sequences are shown in orange on the 3′ side of each gRNA. The exon numbers targeted by the gRNAs are shown below each gRNA. (B) shows validation of STX16 KO, STX17 KO and STX16/STX17 DKO by Western blot analysis in HeLa and Huh7 cell lines. (C) shows WT or STX17KO Huh7 cells were starved with or without the presence of bafilomycin A1 (Baf A1, 100 nM) for 2 h, and cell lysates were subjected to Western blot analysis of LC3B and p62. (D) shows quantifications of LC3B-II levels normalized to β-actin from cells treated as in (C). Data shown as means±SEM of LC3B-II and β-actin ratios, n=3; †, not significant (one-way ANOVA). (E) shows STX16/STX17DKO HeLa cells were transfected with 3×FLAG-STX16, starved with or without the presence of Baf A1 for 2 h, and cell lysates were subjected to Western blot analysis of LC3B. Numbers below the panels are the average of LC3B-II/β-actin ratios normalized to the third lane (HeLa WT treated with EBSS plus Baf A1) from 2 independent experiments. (F) shows WT or STX16/STX17DKO HeLa cells were transfected with mCherry-EYFP-GABARAP (tandem GABARAP), starved in EBSS or EBSS plus Baf A1 for 2 h, and subjected to high-content microscopic (HCM) analysis of autophagic flux for tandem GABARAP. Data shown as means±SEM of the overlap area per cell between mCherry and EYFP, minimum 200 transfected cells were counted each well from at least 12 wells, 3 independent experiments; *, p<0.05; **, p<0.01 (two-way ANOVA). (G) shows representative images of tandem GABARAP transfected into WT or STX16/STX17DKO HeLa cells treated as in (F). Masks: white, cells successfully transfected with tandem GABARAP identified based on average intensity of mCherry; red, mCherry puncta in transfected cells; green, EYFP puncta in transfected cells; yellow, overlap area between mCherry and EYFP. Scale bar: 20 μm.

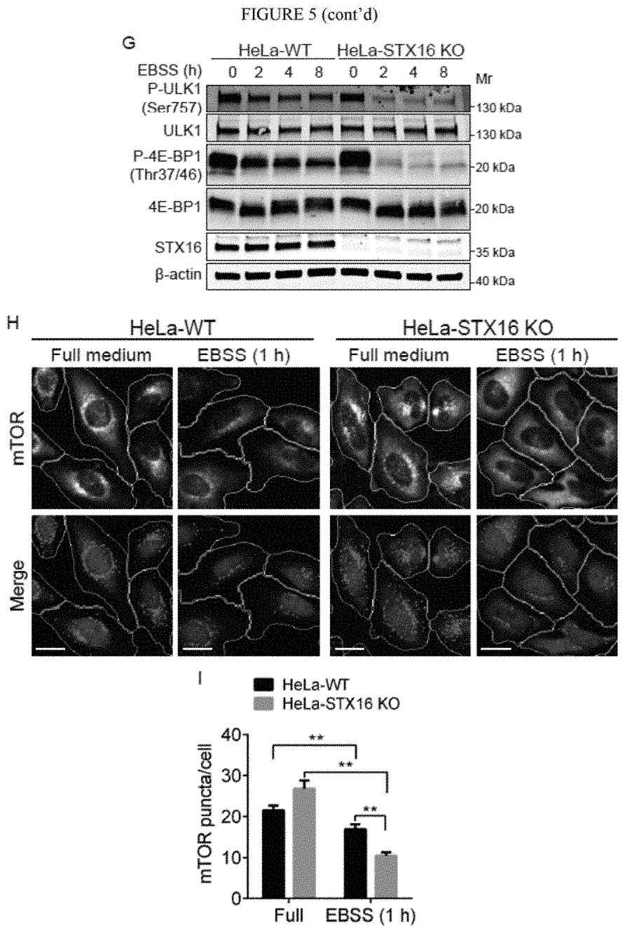

FIGURE EV3 shows that Stx16 is required for lysosomal biogenesis. (A) shows validation of STX16 and STX17 double knockout in HeLa-YFP-Parkin cells by Western blot analysis. Expression of YFP-Parkin was detected by rabbit anti-GFP antibody (ab290). (B) shows western blot analysis of LKB1 expression and AMPKα phosphorylation (Thr172) in response to H₂O₂ treatment in Huh7 cells. (C) shows western blot analysis of Stx16 and Stx17 protein levels in THP1 cells infected with lentiviruses containing STX16, STX17 or STX16 and STX17 CRISPR gRNAs. (D) shows WT car STX16KO HeLa cells were starved in HBSS for 2 h and subjected to HCM analysis of the overlap between TGN46 and LAMP2. Masks: white, cells identified based on nuclei; green, TGN46 indicating trans-Golgi network (TGN); red, LAMP2 puncta; yellow, overlap between TGN46 and LAMP2. Scale bar: 20 μm. (E) shows quantifications of overlaps between TGN46 and LAMP2 in WT and STX16KO HeLa cells treated as in (A). Data shown as means±SEM of percentages of LAMP2 overlapping with TGN46 to compensate reduced LAMP2 puncta in STX16KO cells (see FIG. 5D), minimum 500 cells were counted each well 60m at least 12 wells, 3 independent experiments; **, p<0.01 (two-way ANOVA). (F) shows co-IP analysis of the interactions between overexpressed FLAG-tagged Stx6/Stx16 and endogenous VAMP3/VAMP4/VAMP8/VPS41 in HEK293T cells under full or starved (EBSS for 1 hour) conditions.

FIGURE EV4 shows that Stx16 affects lysosomal homeostasis and mTOR activity. (A) shows WT or STX16KO HeLa cells were starved in EBSS for 6 h and subjected to HCM analysis of the overlap between LAMP2 and mTOR. Masks: white, cells identified based on nuclei; green, LAMP2 puncta; red, mTOR puncta; yellow, overlap between LAMP2 and mTOR. Scale bar 20 μm. (B) shows quantifications of overlaps between LAMP2 and mTOR in WT and STX16KO HeLa cells treated as in (D). Data shown as means±SEM of overlap area per cell between LAMP2 and mTOR (upper panel), and percentages of LAMP2 overlapping with mTOR to compensate reduced LAMP2 puncta in STX16KO cells (see FIG. 5D), minimum 500 cells were counted each well from at least 12 wells, 3 independent experiments: *, p<0.05; **, p<0.01 (two-way ANOVA). (C) shows WT or STX16KO HeLa cells were starved in EBSS for 90 min, fixed and stained with GM130 and TGN46 antibodies, followed by confocal microscopy analysis of the GM130 (Golgi marker, green) and TGN46 (TGN marker, red) profiles. Scale bar, 10 μm (D) shows HCM quantifications of overlaps between GM130 and TGN46 in WT and STX16KO HeLa cells treated as in (A). Data shown as means±SEM of overlap area per cell between GM130 and TGN46; minimum 500 cells were counted each well from at least 12 wells, 3 independent experiments; †, not significant (two-way ANOVA). (E) shows WT or STX16KO HeLa cells were starved in HBSS for 90 min, fixed and stained with M6PR and TGN46 antibodies, followed by HCM analysis of the M6PR and TGN46 profiles. Masks: white, cells identified based on nuclei; green, M6PR puncta; red, TGN46; yellow, overlap between M6PR and TGN46. Scale bar: 20 μm. (F) shows HCM quantifications of overlaps between M6PR and TGN46 in WT and STX16KO HeLa cells treated as in (C). Data shown as means±SEM of overlap area per cell between M6PR and TGN46, minimum 500 cells were counted each well from at least 12 wells, 3 independent experiments; †, not significant (two-way ANOVA).

FIGURE EV5 shows that mAtg8s affect acidification of the endolysosomal system and mTOR activity. (A) shows Western blot analysis of LC3 (LC3A,B,C), GABARAP, GABARAPL1 and GABARAPL2 in WT HeLa or cells knocked out for LC3A,B,C (LC3 TKO), GABARAP, -L1, -L2 (GBRP TKO) or all 6 mATG8s (Hexa KO). (B) shows HeLa cells were transfected with WT or LIR-mutant EGFP-tagged Stx16, and subjected to HCM analysis of overlaps between Stx16 and LAMP2. Masks: white, cells transfected with Stx16 identified based on the average intensity of EGFPStx16; green, EGFP-Stx16 puncta; red, LAMP2 puncta; yellow, overlap between EGFP-Stx16 and LAMP2. Scale bar: 20 μm. (C) shows HCM quantifications of overlaps between LAMP2 and WT or different types of LIRmutant EGFP-Stx16 transfected into HeLa cells and treated as in (B). Data shown as means±SEM of percentages of EGFP-Stx16 overlapping with LAMP2, minimum 200 transfected cells were counted each well from at least 12 wells, 3 independent experiments; **, p<0.01 (one-way ANOVA). (D) shows endogenous Co-IP analysis of the interactions between Stx16, Vti1a and Stx6 in WT, LC3 TKO, GABARAP TKO or Hexa KO HeLa cells. * indicates mouse IgG of precipitated mouse anti-Stx6 antibody. (E) shows endogenous Co-IP analysis of the interactions between VPS41, Stx16, Vti1a and Stx6 in WT, or ATG3 KO cells. * indicates mouse IgG of precipitated mouse anti-Stx6 antibody. (F) shows WT or Hexa KO HeLa cells were starved in EBSS for indicated time points, and cell lysates were subjected to Western blot analysis of mTOR activity by mTOR substrate phosphorylation.

FIGURE S1 shows that Stx16 is required for lysosomal biogenesis. (A) shows STX16/STX17^{DKO} HeLa cells were transfected with EGFP vector or EGFP-Stx16 (WT or LIR-mutant) and starved in HBSS for 2 h, and subjected to HCM analysis of LAMP2 puncta. Masks: white, cells expressing EGFP or EGFP-Stx16 identified based on average intensity of EGFP; green, EGFP-Stx16 puncta; red, LAMP2 puncta; yellow, overlap between EGFP-Stx16 and LAMP2. Scale bar: 20 μm. (B) shows quantifications of LAMP2 puncta in WT or STX16/STX17^{DKO} HeLa cells transfected with WT or LIR-mutant EGFP-Stx16 and treated as in (A). Data, means±SEM of LAMP2 puncta per cell, minimum 200 transfected cells were counted each well from at least 12 wells, 3 independent experiments; **, p<0.01 (one-way ANOVA). (C) shows endogenous Co-IP analysis of the interactions between VPS41, VPS33A, Vti1a and Stx6 in HeLa cells. * indicates mouse IgG of precipitated mouse anti-Stx6 antibody.

FIGURE S2 shows that Stx16 is required for mTOR puncta reformation after long-term starvation. (A) shows WT or STX16^{KO} HeLa cells were starved in EBSS for 6 h and subjected to HCM analysis of mTOR puncta. Masks: white, cells identified based on nuclei; red, mTOR puncta. mTOR puncta and quantifications for 1 h starvation are shown in FIGS. 5H and I. Scale bar: 20 μm. (B) shows quantifications of mTOR puncta in WT and STX16^{KO} cells treated as in (A). Data, means±SEM of mTOR puncta per cell, minimum 500 cells were counted each well from at least 12 wells, 3 independent experiments; †, not significant; **, p<0.01 (two-way ANOVA).

FIGURE S3 shows Stx16 knockout does not affect acidification of the endolysosomal system. (A) shows WT or STX16^{KO} HeLa cells were starved in EBSS for 1 h, followed by starvation in the presence of LysoTracker Green DND-26 (LTG) for additional 30 min, and subjected to ImageStream flow cytometry (Amnis) analysis of LTG profiles. (B) shows quantifications of LTG counts per cell in WT and STX16^{KO} HeLa cells treated as in (A) by IDEAS software from Amnis analysis. (C) shows representative images of LTG profiles in WT and STX16^{KO} HeLa cells treated as in (A). Channel 01 shows bright field, and Channel 02 shows LysoTracker Green with the excitation wavelength at 488 nm.

FIGURE S4 shows the effects of mATG8s knockout on lysosomal biogenesis. (A) shows HCM analysis of LAMP2 puncta in WT, LC3^{TKO}, GABARAP^{TKO} or Hexa^{KO} HeLa cells under frill medium or starved conditions (EBSS for 2 h). Masks: white, cells identified based on nuclei; green, LAMP2 puncta. Scale bar: 20 μm. (B) shows quantifications of LAMP2 puncta in WT, LC3^{TKO}, GABARAP^{TKO} or Hexa^{KO} HeLa cells treated as in (A). Data, means±SEM of LAMP2 puncta (upper panel) or LAMP2 total area (lower panel) per cell, minimum 600 cells were counted each well from at least 12 wells, 3 independent experiments; †, not significant; *, p<0.05 (two-way ANOVA). (C) shows western blot analysis of LAMP1 and LAMP2 protein levels in WT, LC3^{TKO}, GABARAP^{TKO} and Hexa^{KO} HeLa cells starved in HBSS for 2 h. (D) shows western blot analysis of LAMP1 and LAMP2 protein levels in WT, LC3^{TKO}, GABARAP^{TKO} and Hexa^{TKO} HeLa cells starved in HBSS for 8 h.

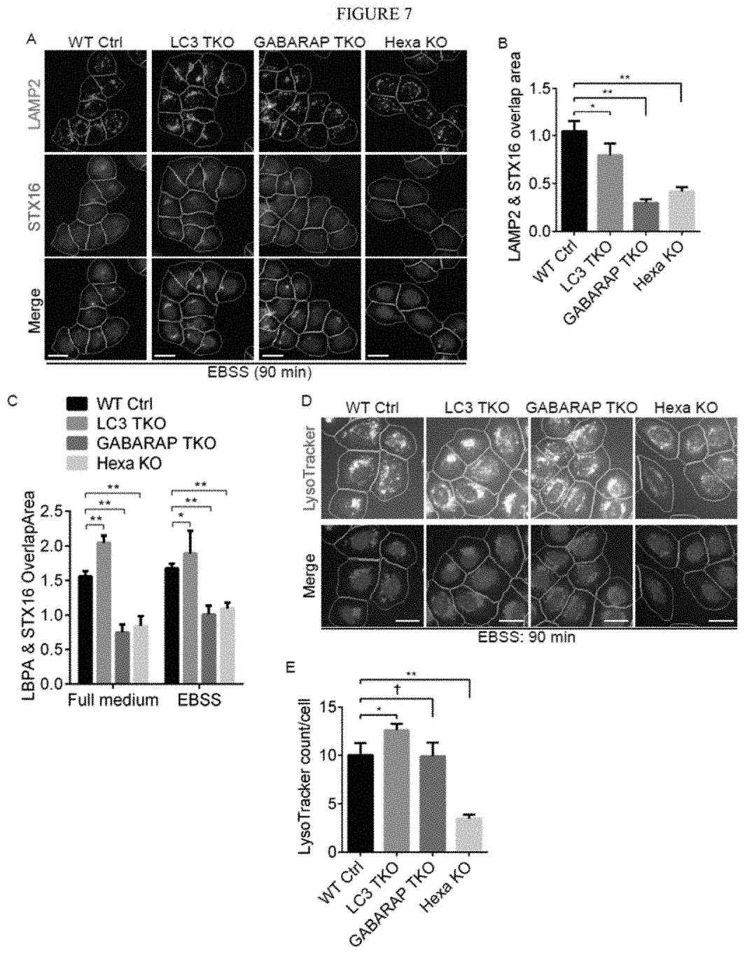

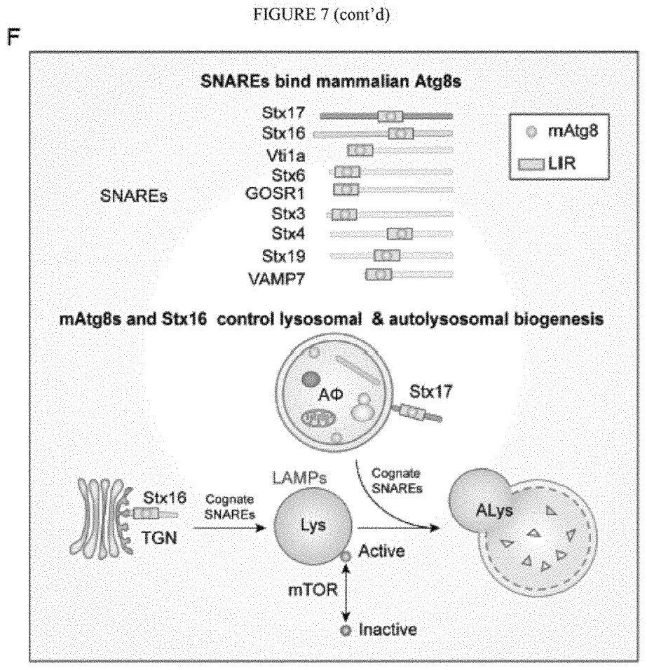

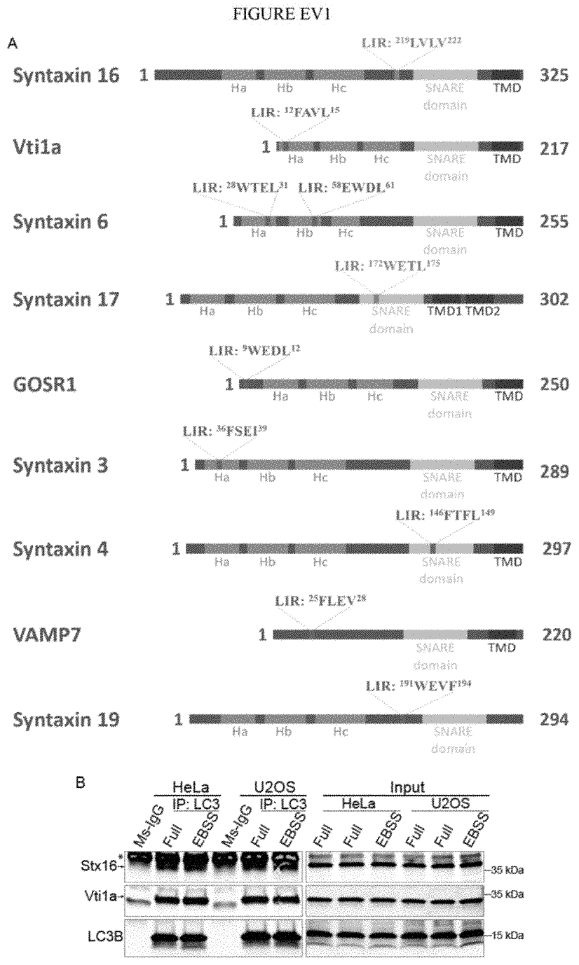

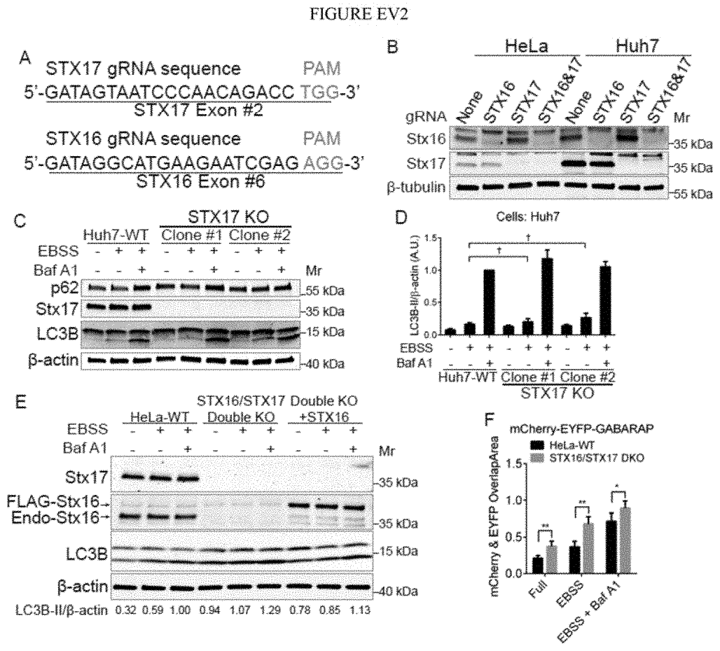

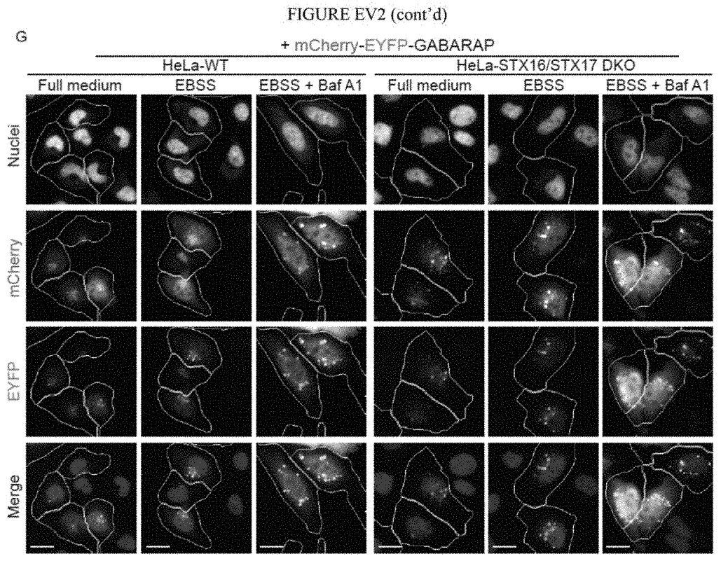

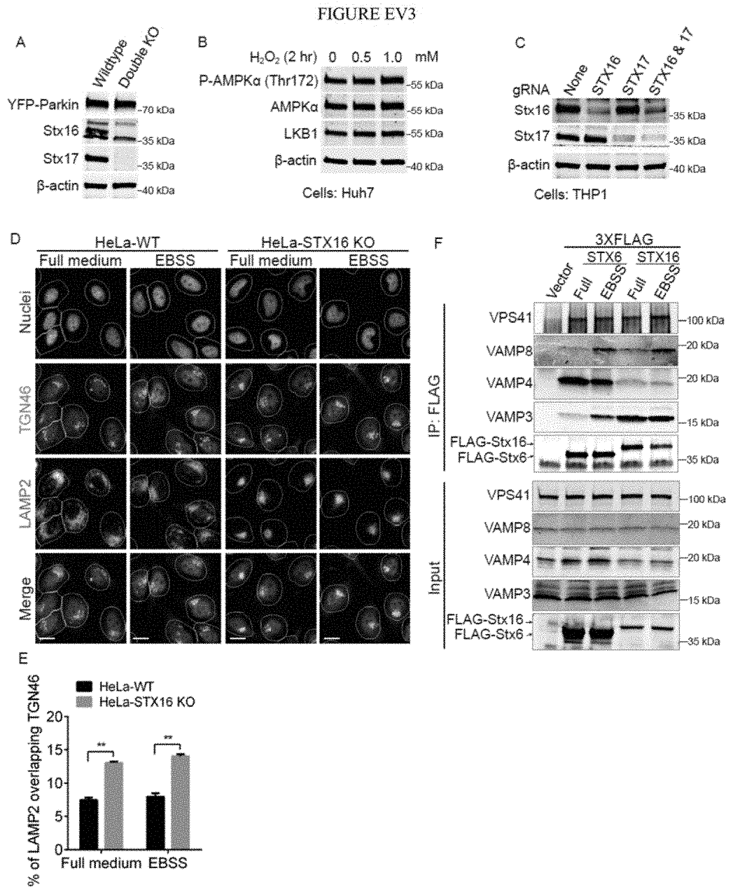

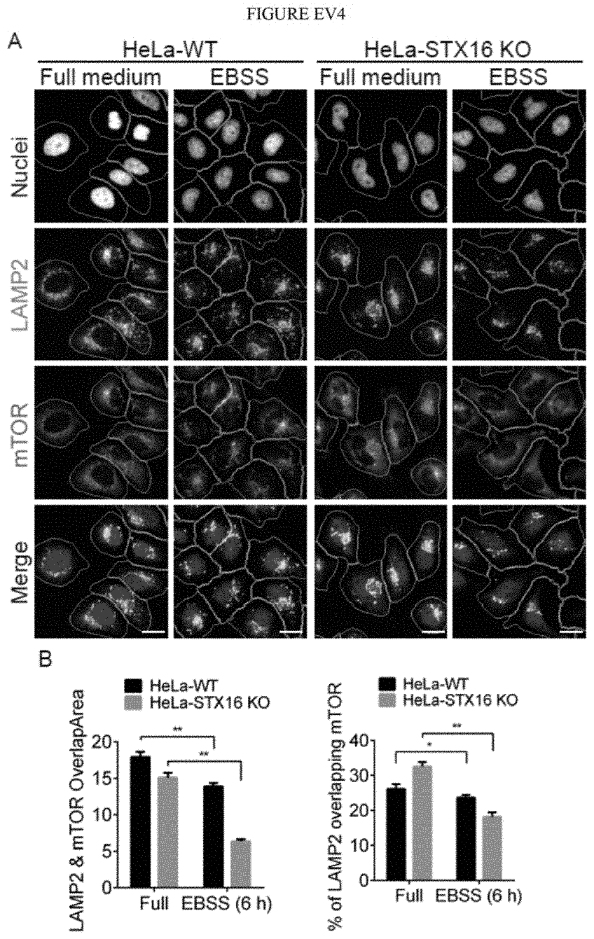

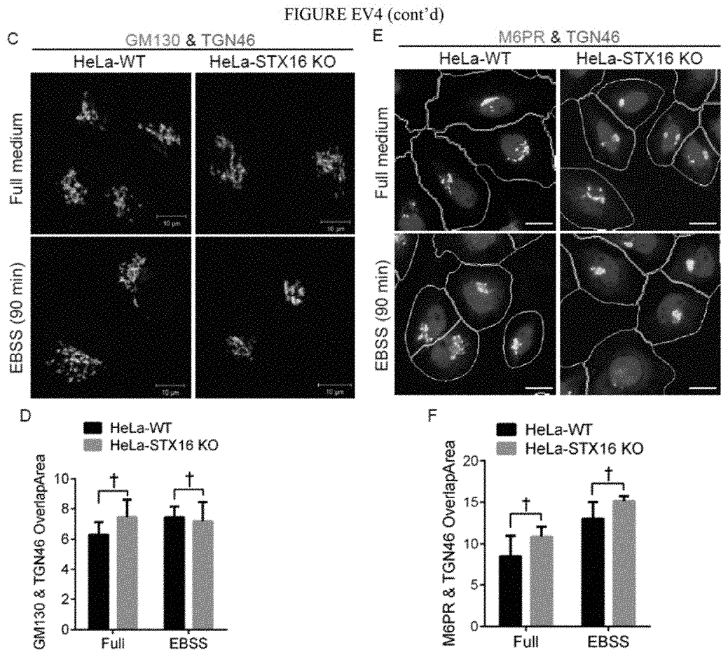

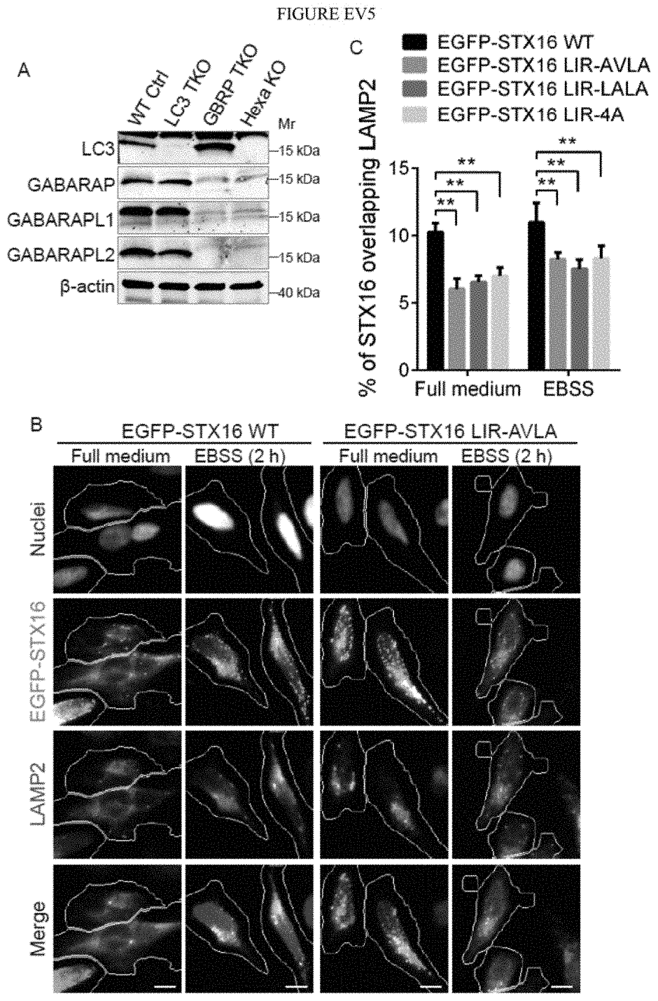

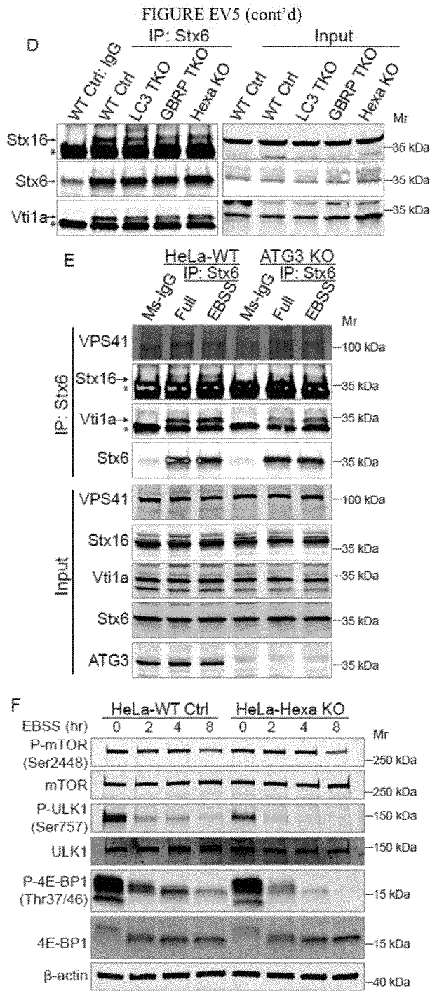

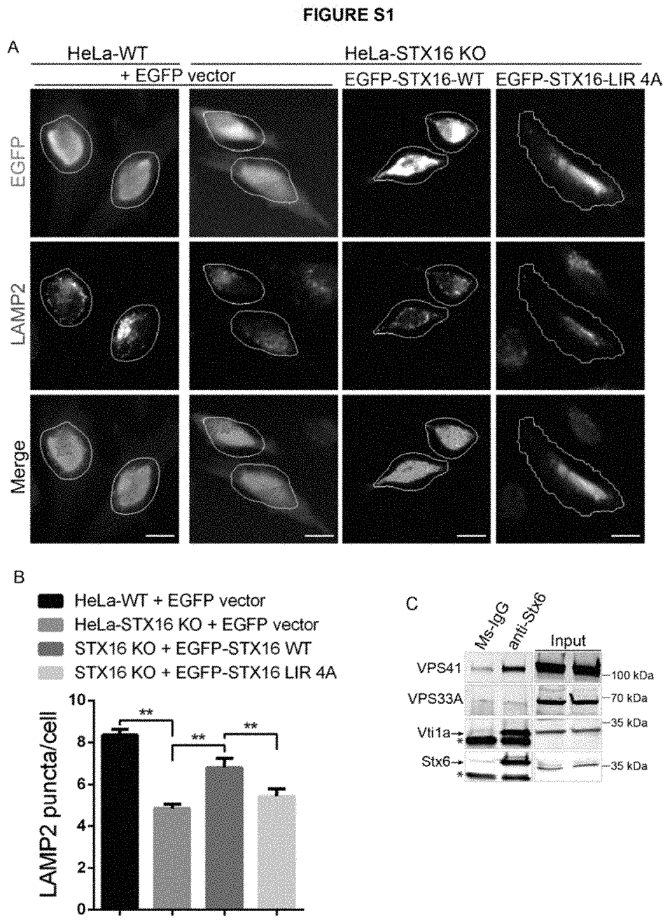

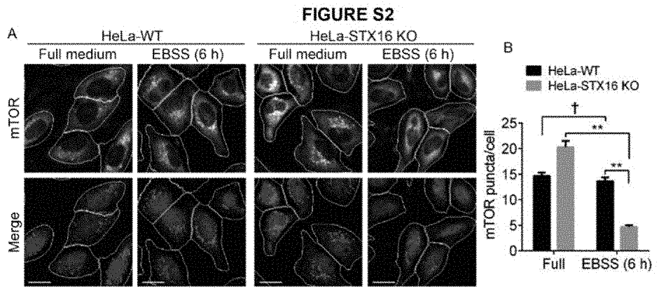

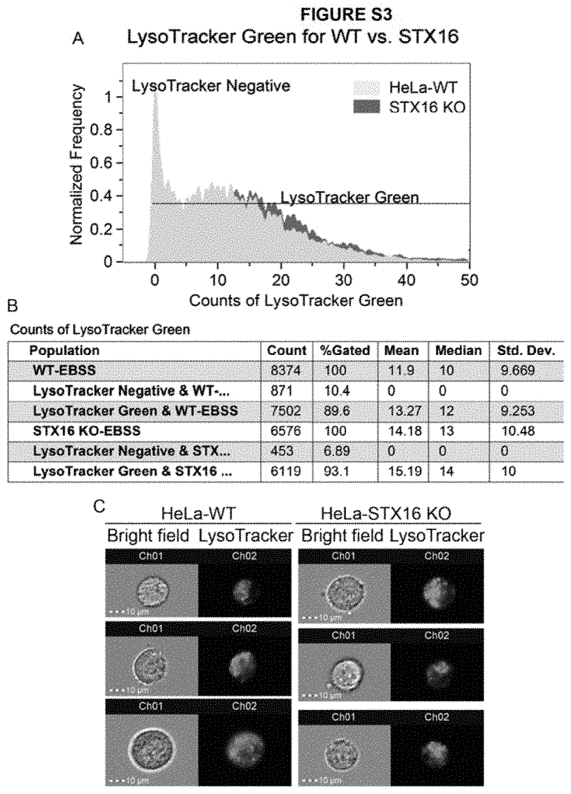

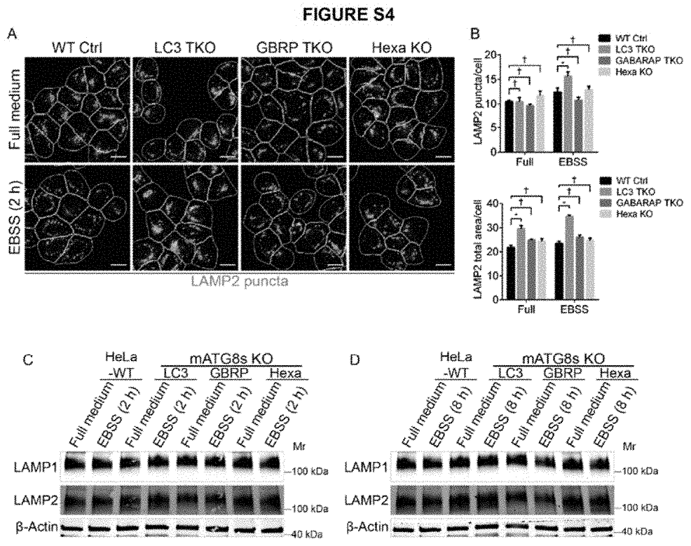

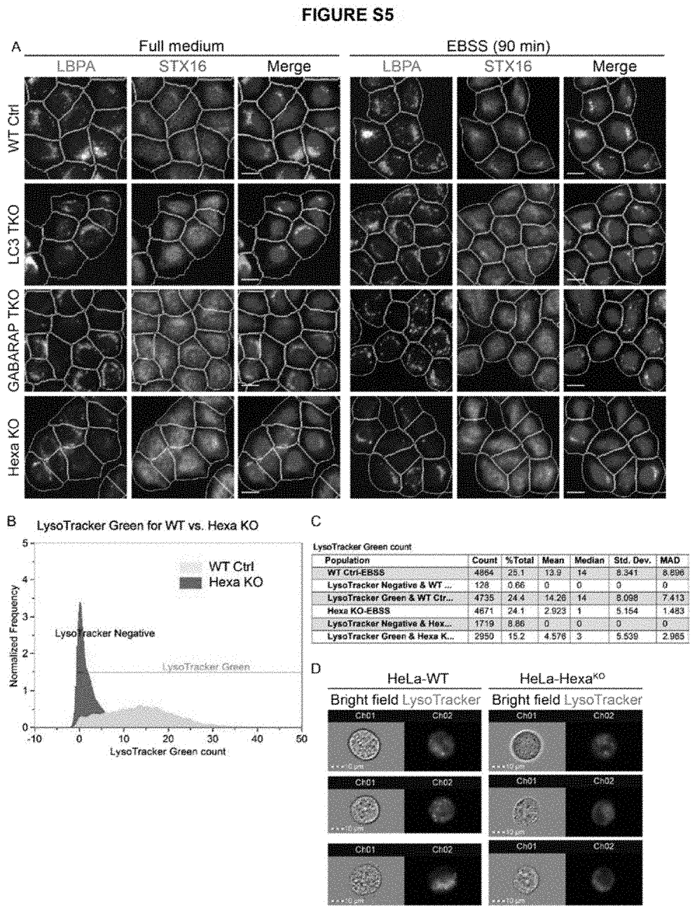

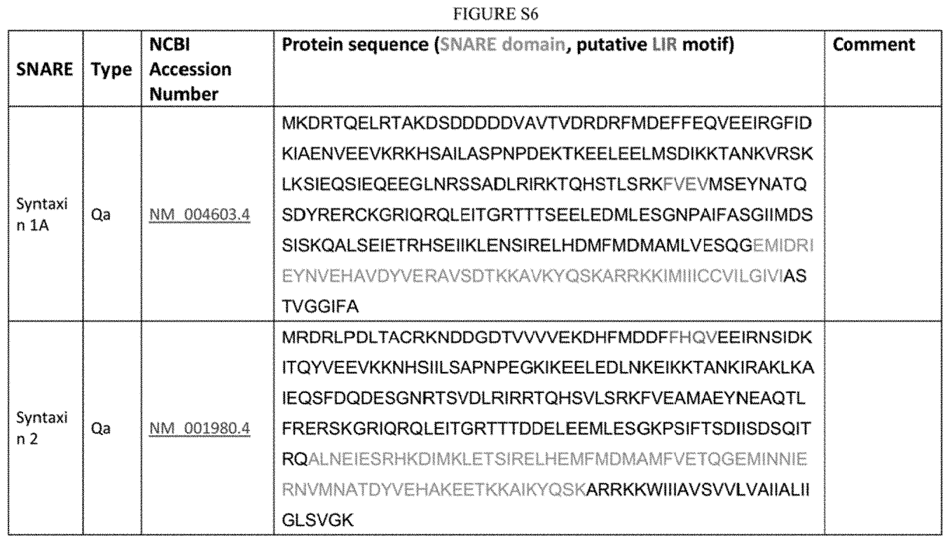

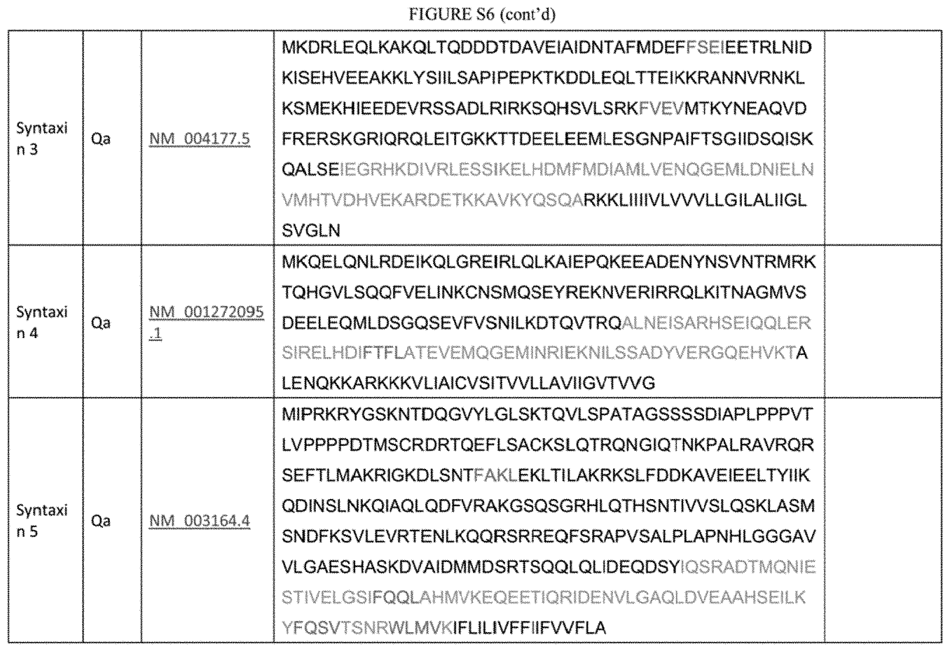

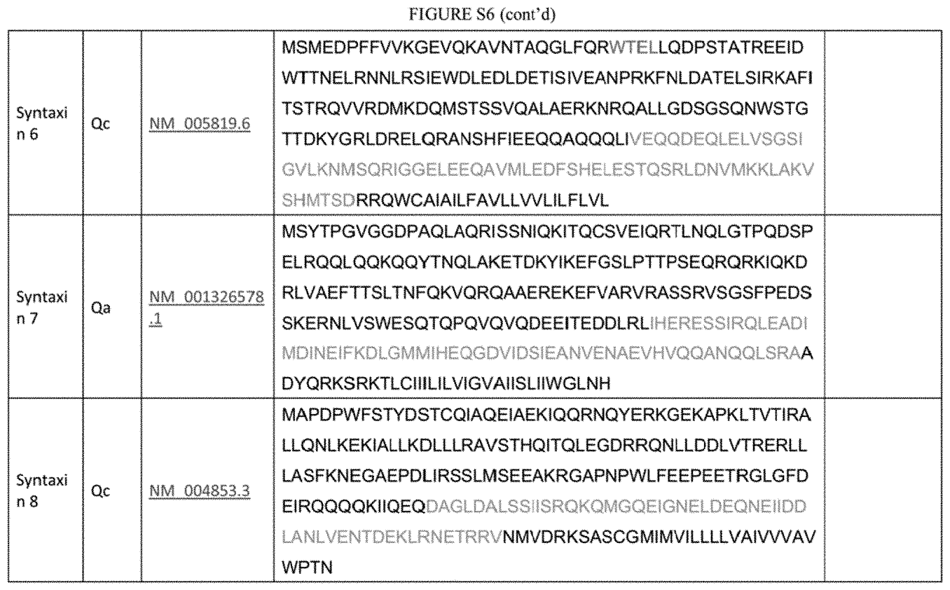

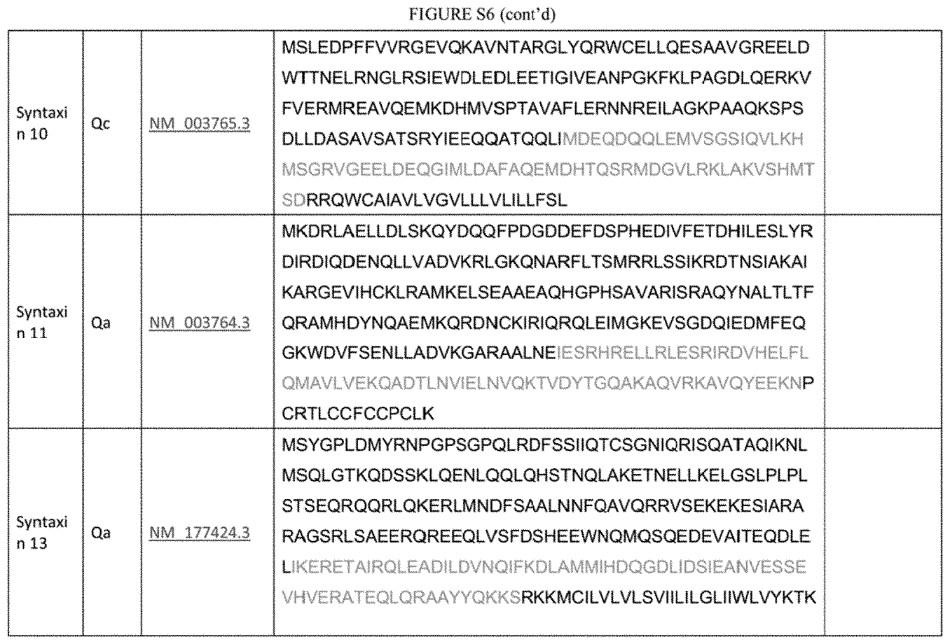

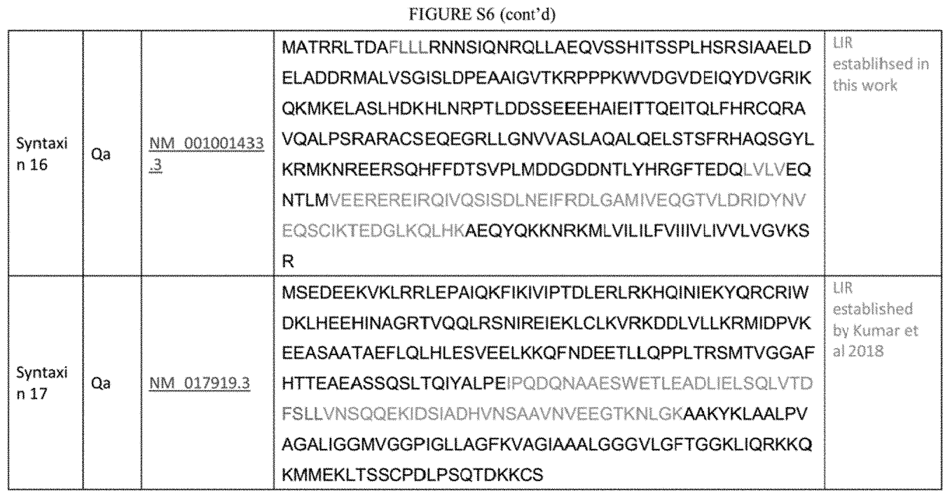

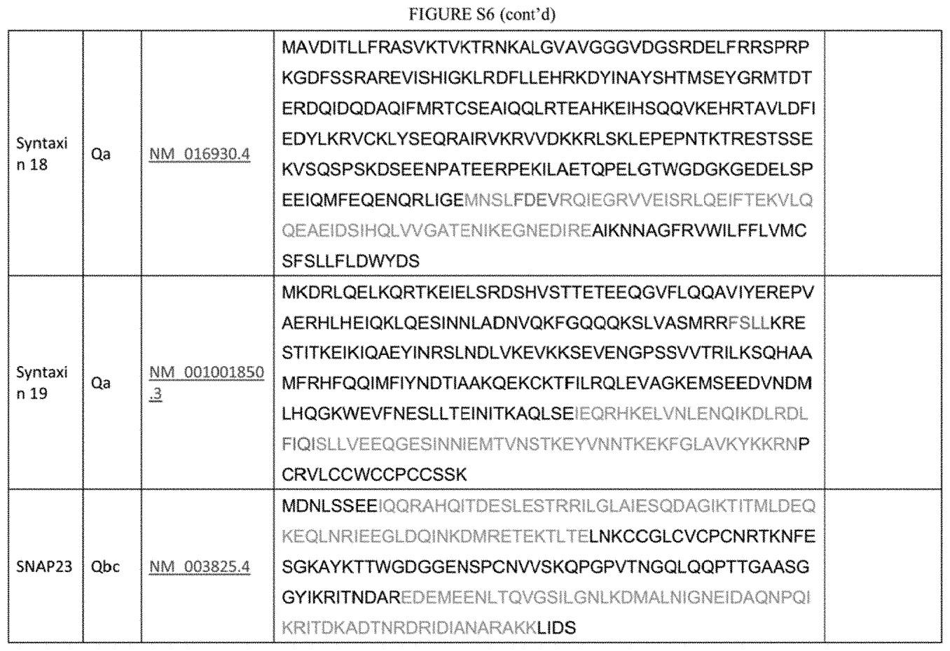

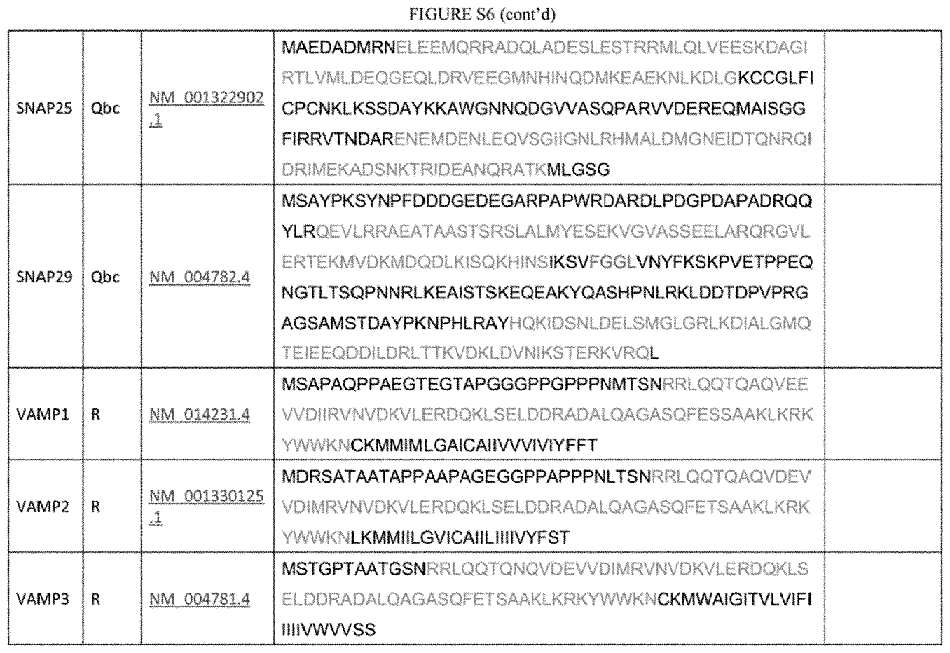

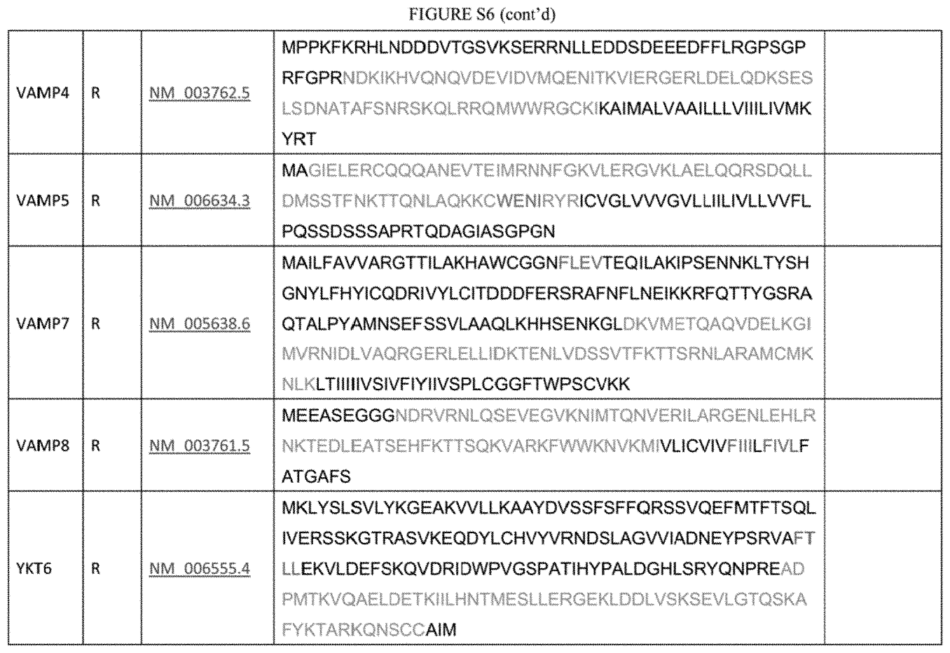

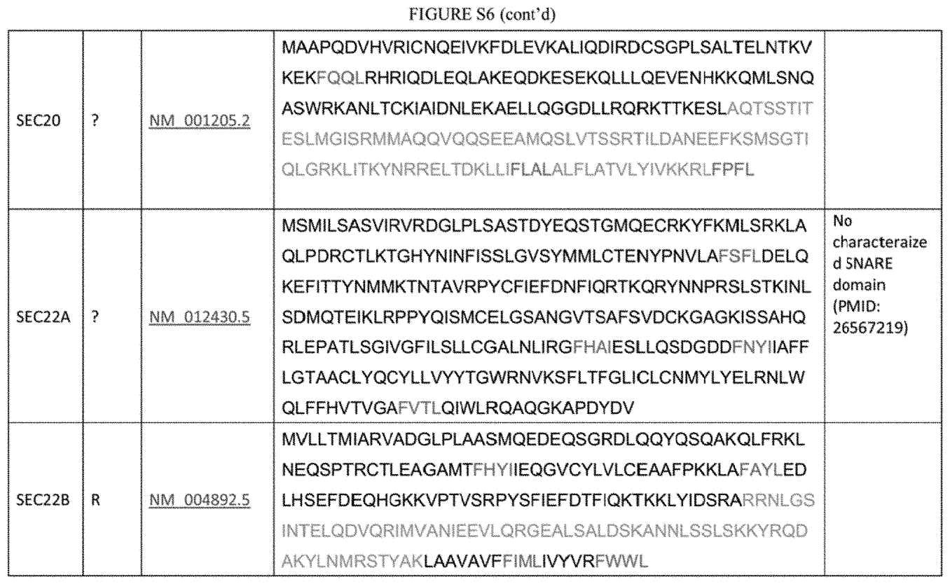

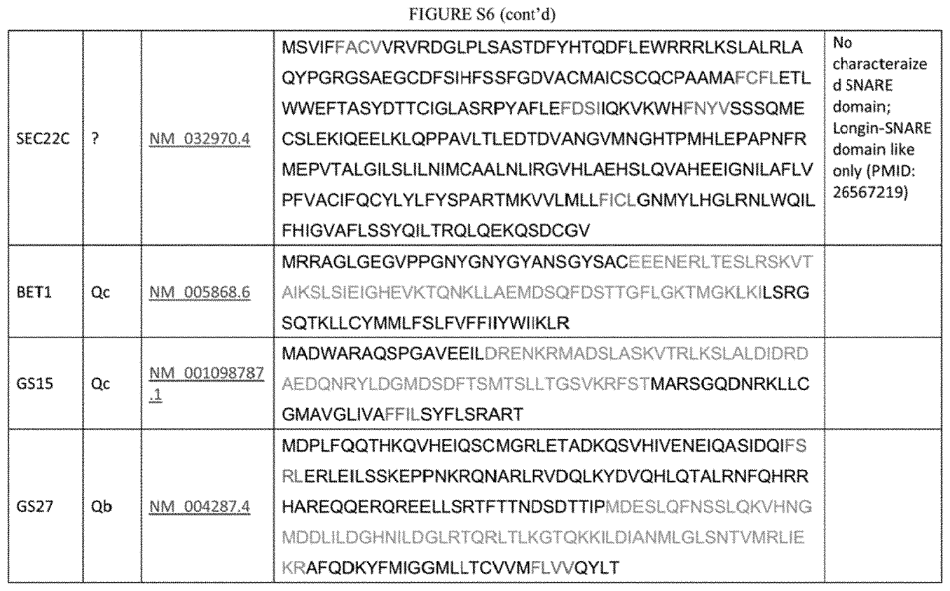

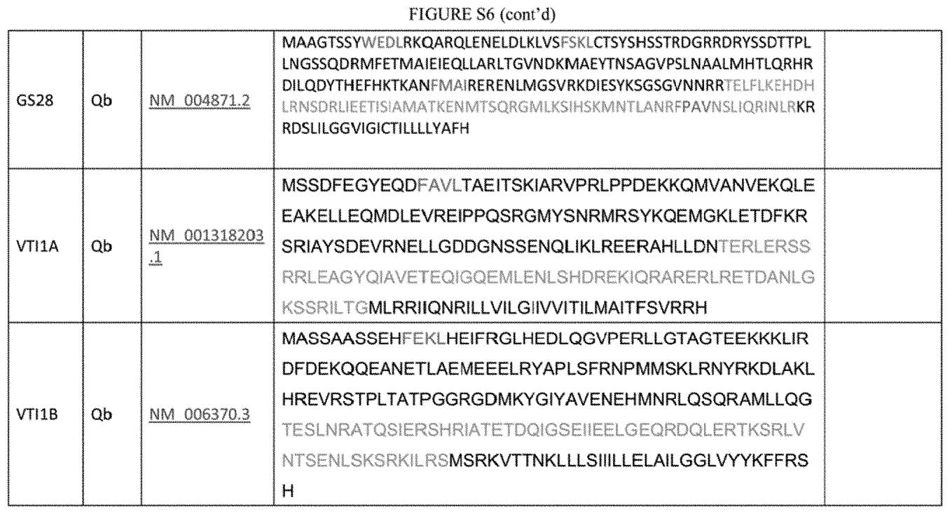

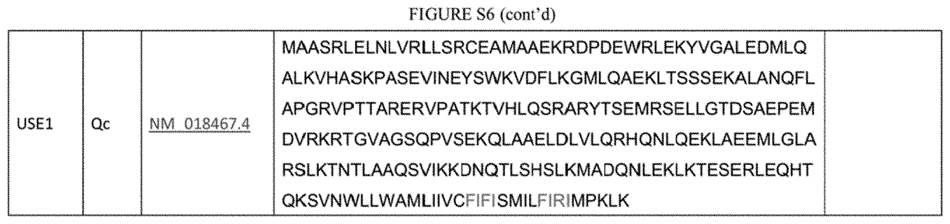

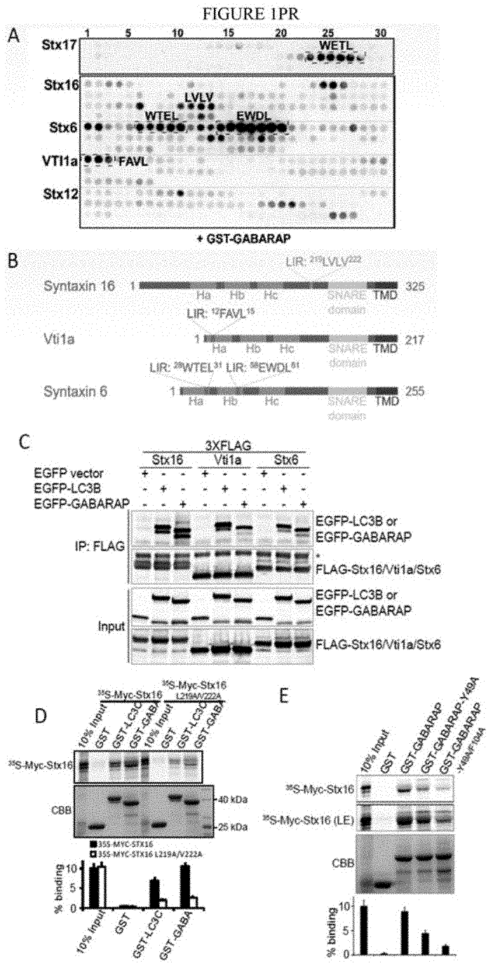

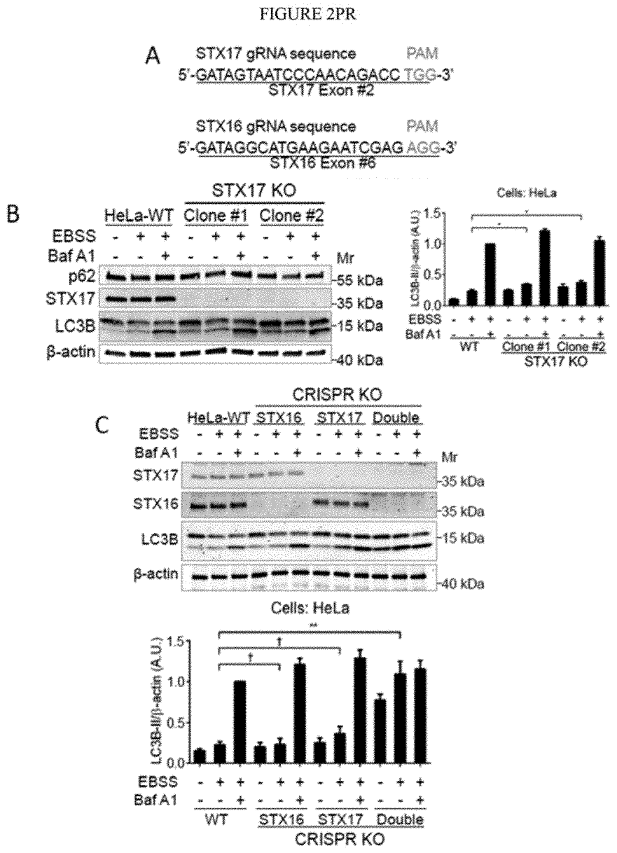

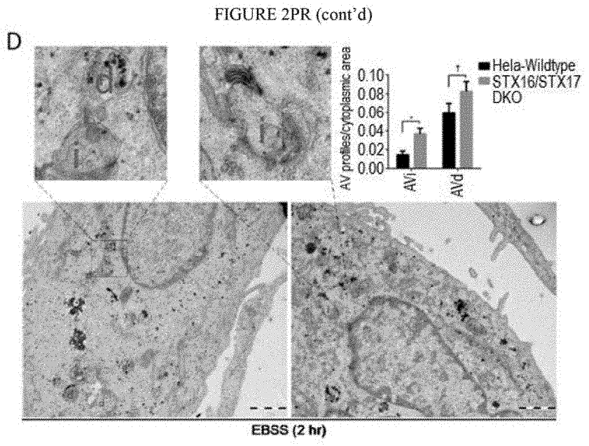

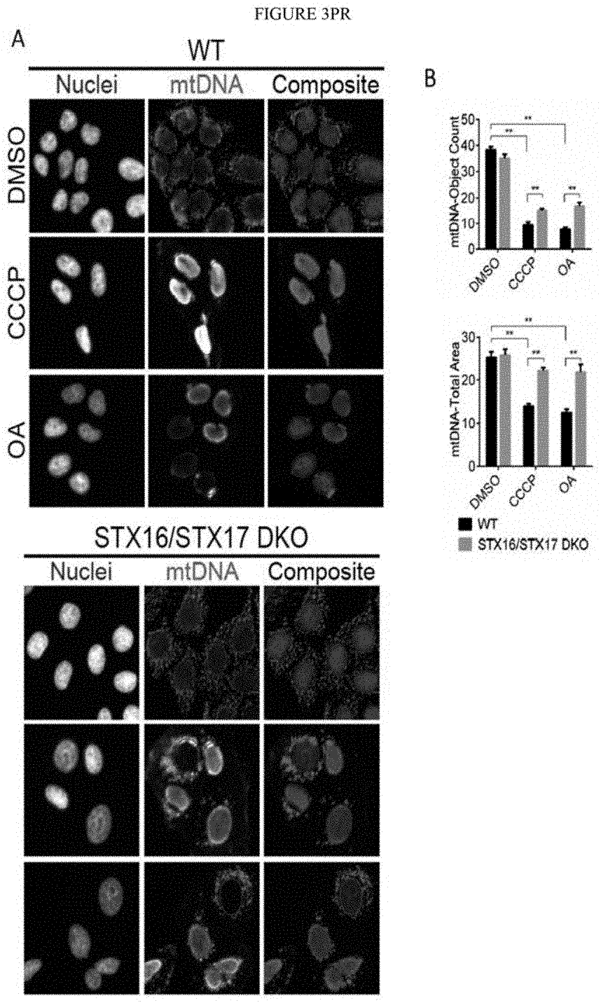

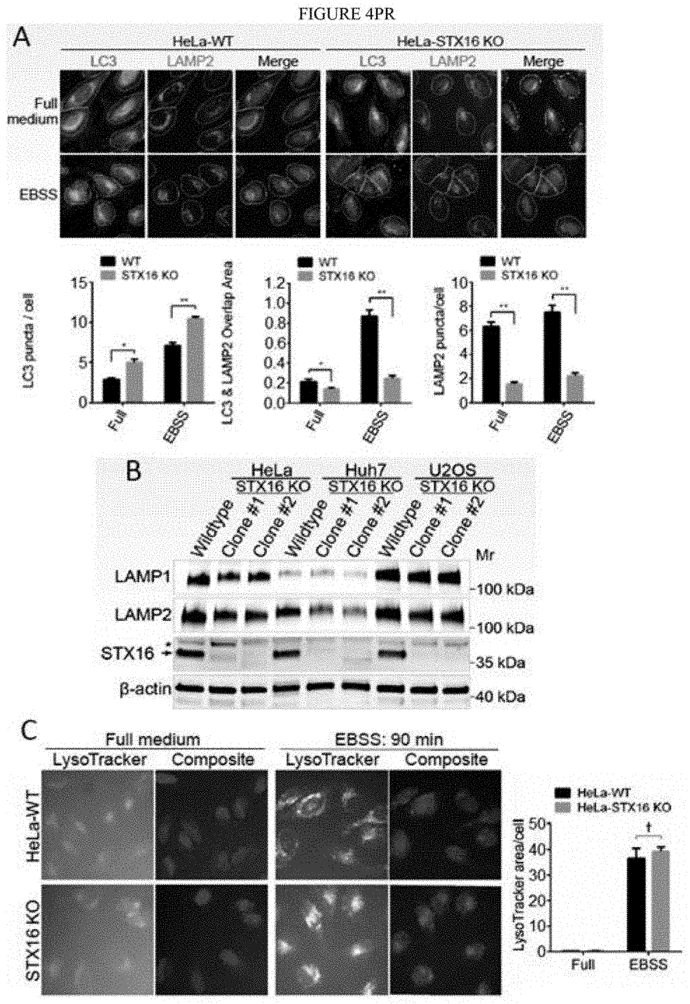

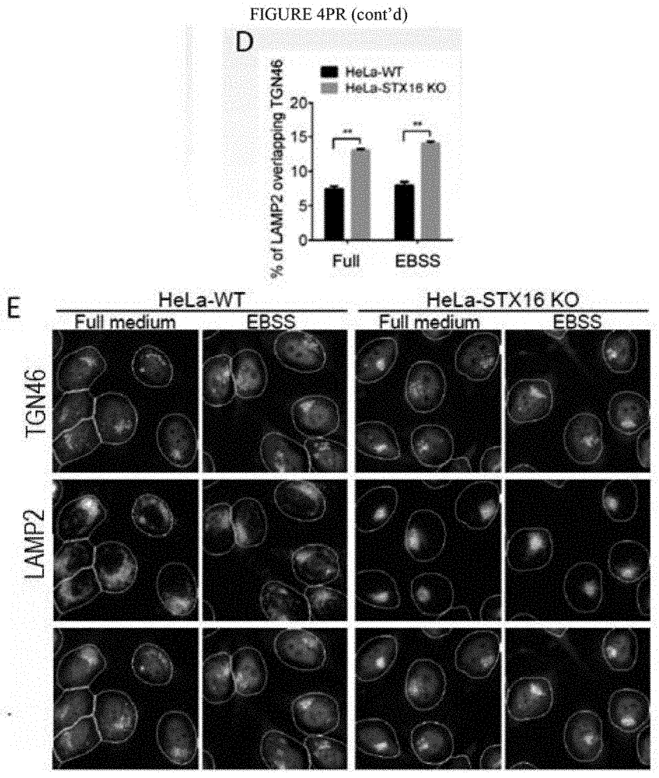

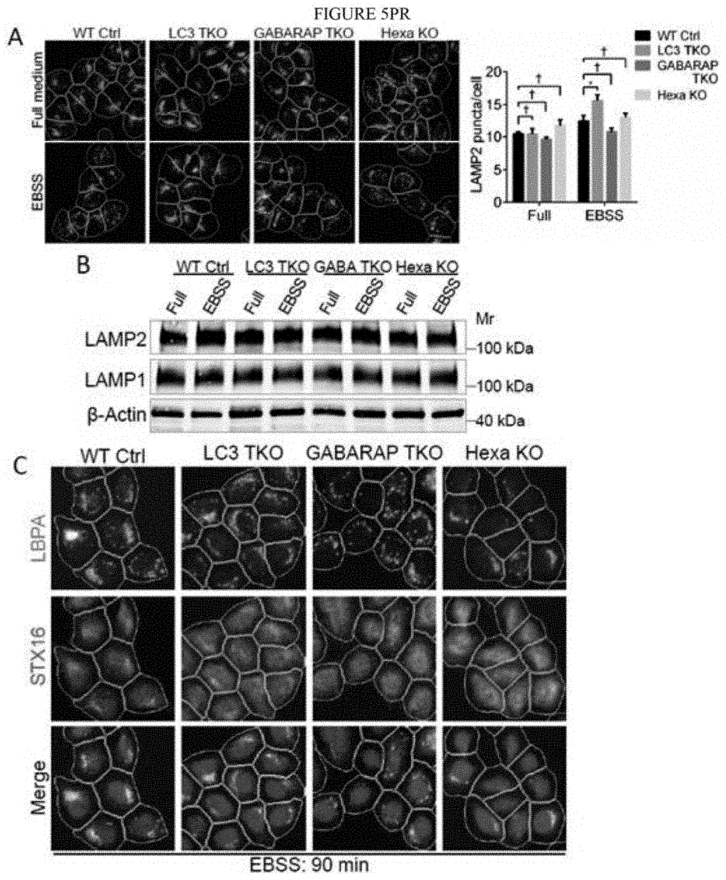

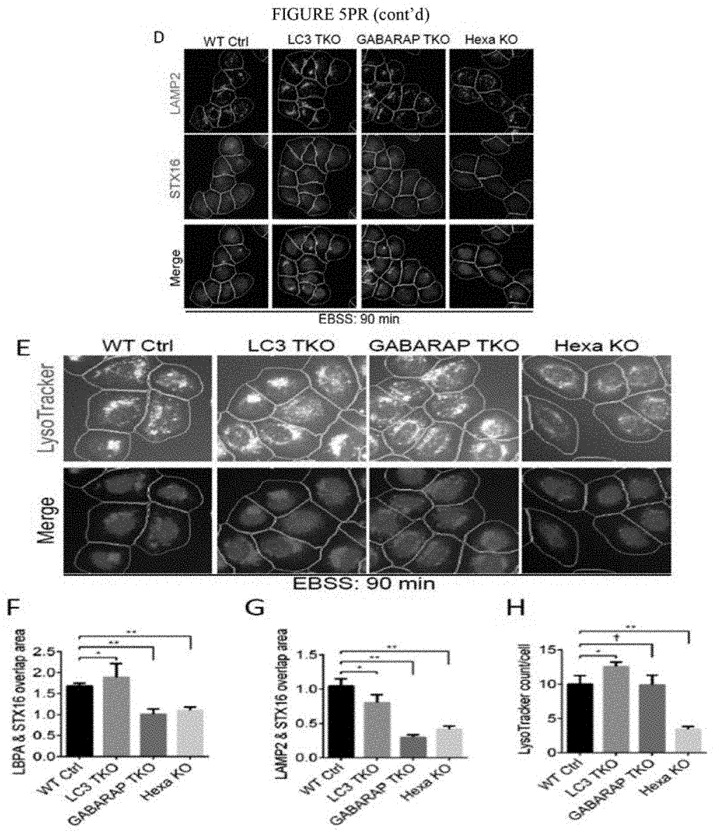

FIGURE SS shows that mAtg8 proteins regulate proper localization of Stx16 and acidification of the endolysosomal system. (A) shows WT, LC3^{TKO}, GABARAP^{TKO} or Hexa^{TKO} HeLa cells were grown in full medium or starved in EBSS for 90 min and subjected to HCM analysis of overlaps between LBPA and Stx16. Masks: white, cells identified based on nuclei; green, LBPA puncta; red, Stx16 puncta; yellow, overlap of LBPA and Stx16. Quantifications of the overlaps between LBPA and Stx16 are shown in FIG. 7C. Scale bar: 20 μm. (B) shows WT or Hexa^{KO} HeLa cells were starved in EBSS for 1 hour, followed by starvation in the presence of LysoTracker Green DND-26 (LTG) for additional 30 min, and subjected to ImageStream flow cytometry (Amnis) analysis of LTG profiles. (C) shows quantifications of LTG counts/cell in WT and Hexa^{KO} HeLa cells treated as in (B) by IDEAS software from Amnis analysis. (D) shows representative images of LTG profiles in WT and Hexa^{KO} HeLa cells treated as in (B). Channel 01 shows bright field, and Channel 02 shows LysoTracker Green with the excitation wavelength at 488 nm.

FIGURE S6 shows candidate LIR (LC3-interacting region) motifs. This Table provides a global SNARE sequence analysis for the presence of typical LIR motifs according to the consensus sequences described in Birgisdottir et al., 2013. Blue foot, SNARE domain; Red font, putative LIR motif; Green, experimentally established LIR.

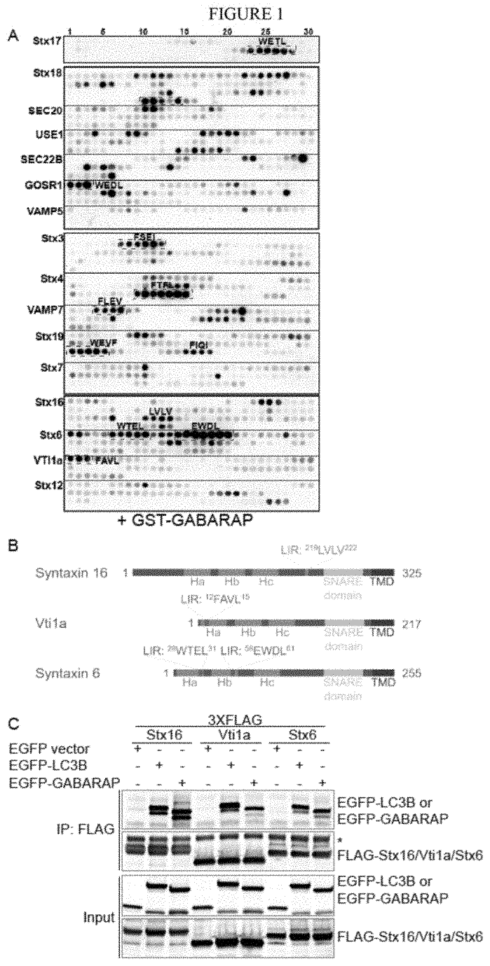

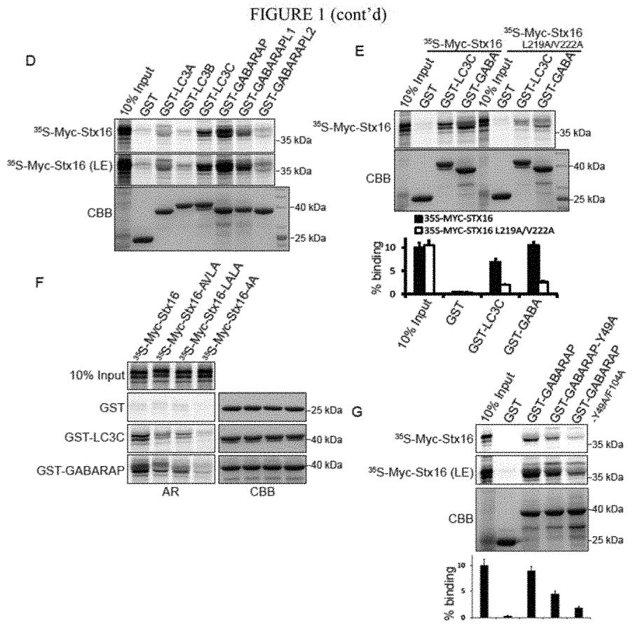

FIG. 1PR shows that Stx16 interacts with mAtg8s through LIR motif. (A) Peptide arrays identify LIR motifs in the indicated SNARE proteins. (B) Schematics of the functional domains of Stx16/Vti1a/Stx6 cognate SNAREs with the positions of LIR motifs. (C) Co-IP analysis of the interactions between Stx16/Vti1a/Stx6 and LC3B or GABARAP. (D) GST pull-down analysis of interactions between WT or LIR-mutant Stx16 and GST-tagged LC3C or GABARAP. (E) GST pull-down analysis of interactions between Stx16 and WT or LDS-mutant GABARAP.

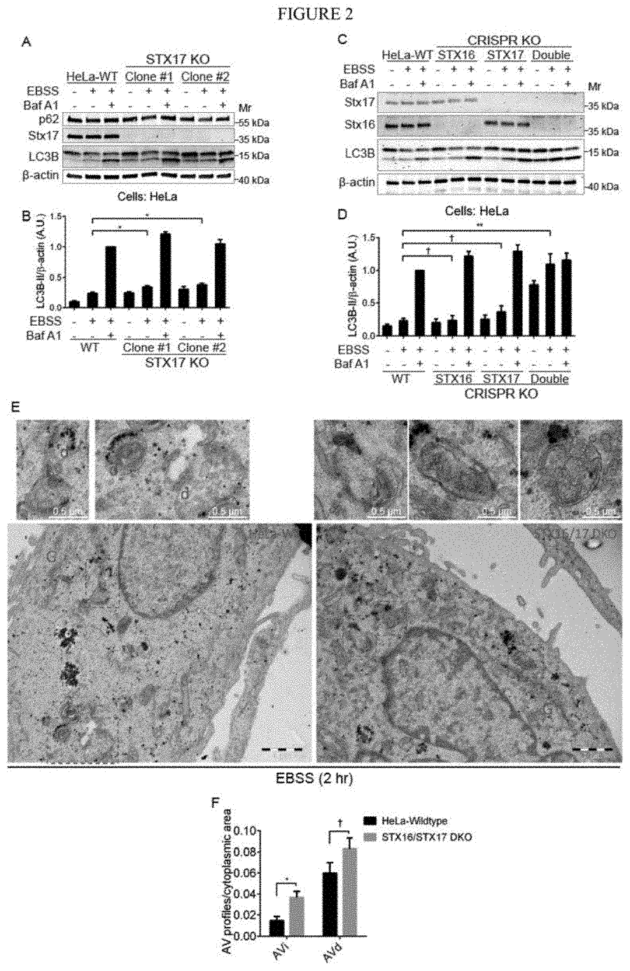

FIG. 2PR shows that Stx16 and Stx17 cooperate to promote autophagic flux induced by bulk autophagy. (A) Sequences for gRNAs targeting STX16 and STX17. (B) Autophagic flux continues in STX17 KO cells; right graph shows quantification for LC3B-II/β-Actin. (C) STX16/STX17 DKO inhibits starvation-induced autophagic flux. (D) Electron microscopy showing increased accumulation of initial autophagic vesicles (AVi) in STX16/STX17 DKO cells.

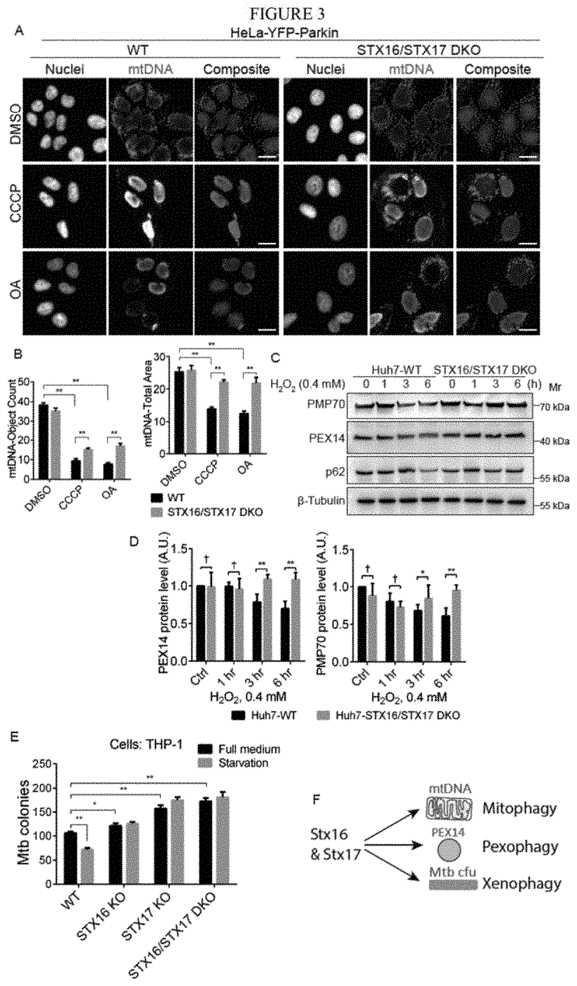

FIG. 3PR shows that STX16/STX17 DKO inhibits mitophagy. (A) High-content images for mitochondria DNA (mtDNA) in WT or STX16/STX17 DKO HeLa-YFP-Parkin cells were treated with CCCP or oligomycin A plus antimycin A (OA) for 16 hours. (B) Quantifications of mtDNA from cells treated as in (A)

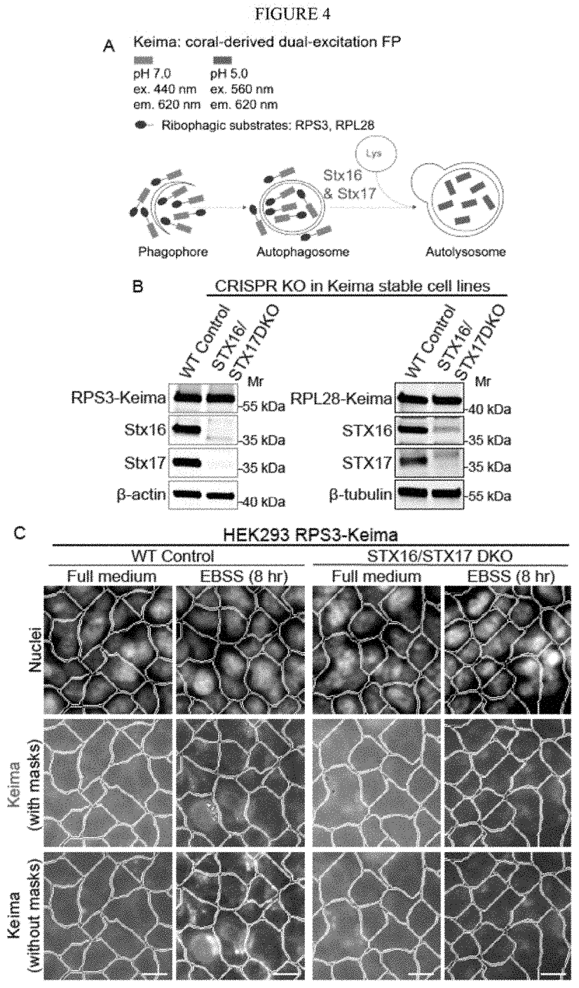

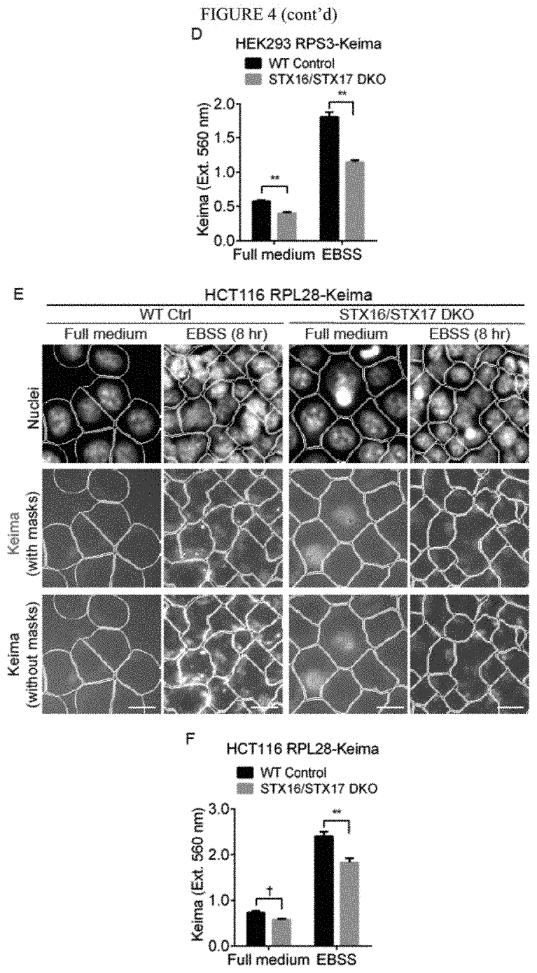

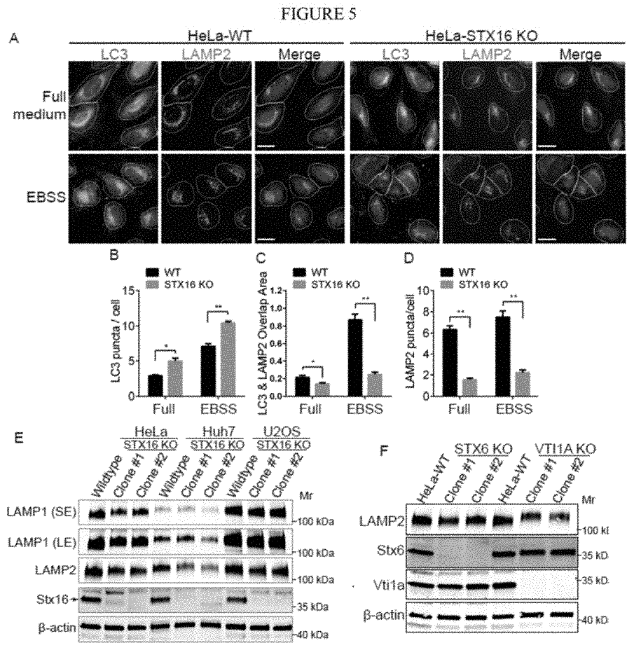

FIG. 4 PR shows that Stx16 functions in the delivery of lysosomal membrane proteins. (A) High-content images show reduced LAMP2 pucnta in STX16-KO cells. (B) Western blot analyses of LAMP1/2 in HeLa, Huh7 and U2OS cells knocked out for STX16. (C) High-content images of LysoTracker staining in WT and STX16-KO HeLa cells. (D,E) High-content microscopy analysis shows increased overlap between LAMP2 and TGN46 in STX16-KO cells.

FIG. 5PR shows that mAtg8s regulate proper localization of Stx16 and control endolysosomal system in the cell. (A & B) Knockout of mATG8s does not reduced the abundance of lysosomal membrane proteins. (C) Knockout of mATG8s reduces the overlap between Stx16 and LBPA; quantification in (F). (D) Knockout of mATG8s reduces the overlap between Stx16 and LAMP2; quantification in (G). (E) High-content microscopy analysis of LysoTracker Red shows reduced acidified compartments in cells knocked out for all 6 mATG8s; quantification in (H).

### DETAILED DESCRIPTION OF THE INVENTION

It is noted that, as used in this specification and the appended claims, the singular forms “a,” “an,” and “the,” include plural referents unless expressly and unequivocally limited to one referent. Thus, for example, reference to “a compound” includes two or more different compound. As used herein, the term “include” and its grammatical variants are intended to be non-limiting, such that recitation of items in a list is not to the exclusion of other like items that can be substituted or other items that can be added to the listed items.

The term “compound” or “agent”, as used herein, unless otherwise indicated, refers to any specific chemical compound or composition (such as an inhibitor or agonist of Atg8, Stx 16, Stx 17, Galectin-8 or Galectin-9, galactose, another mTOR inhibitor and/or a lysosomotropic agent and/or an autophagy modulator agent) disclosed herein and includes tautomers, regioisomers, geometric isomers as applicable, and also where applicable, stereoisomers, including diastereomers, optical isomers (e.g. enantiomers) thereof as well as pharmaceutically acceptable salts or alternative salts thereof. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds as well as diastereomers and epimers, where applicable in context. The term also refers, in context to prodrug forms of compounds which have been modified to facilitate the administration and delivery of compounds to a site of activity.

The term “patient” or “subject” is used throughout the specification within context to describe an animal, generally a mammal, including a domesticated mammal including a farm animal (dog, cat, horse, cow, pig, sheep, goat, etc.) and preferably a human, to whom treatment, including prophylactic treatment (prophylaxis), with the methods and compositions according to the present invention is provided. For treatment of those conditions or disease states which are specific for a specific animal such as a human patient, the term patient refers to that specific animal, often a human.

The terms “effective” or “pharmaceutically effective” are used herein, unless otherwise indicated, to describe an amount of a compound or composition which, in context, is used to produce or affect an intended result, usually the modulation of autophagy within the context of a particular treatment or alternatively, the effect of a bioactive agent which is coadministered with the autophagy modulator (autotoxin) in the treatment of disease.

The terms “treat”, “treating”, and “treatment”, etc., as used herein, refer to any action providing a benefit to a patient at risk for or afflicted by an autophagy mediated disease state or condition as otherwise described herein. The benefit may be in curing the disease state or condition, inhibiting its progression, or ameliorating, lessening or suppressing one or mare symptom of an autophagy mediated disease state or condition, especially including excessive inflammation caused by the disease state and/or condition. Treatment, as used herein, encompasses therapeutic treatment and in certain instances, prophylactic treatment (i.e., reducing the likelihood of a disease or condition occurring), depending on the context of the administration of the composition and the disease state, disorder and/or condition to be treated.

The term “ATg8” is used to describe autophagy-related protein 8, most often human ATg8. Autophagy-related (“Atg”) proteins are eukaryotic factors participating in various stages of the autophagic process. Autophagy-related protein (Atg) is a ubiquitin-like protein required for the formation of autophagosomal membranes. The transient conjugation of Atg to the autophagosomal membrane through a ubiquitin-like conjugation system is essential for autophagy in eukaryotes. Thus, this protein represents a potential target for influencing autophagy in subjects and for treating autophagy mediated disease states and/or conditions. Atg8 in humans has several proteins which exist in a subfamily of Atg8 proteins. These include GABA Type A receptor associated protein type 2 (GABARAPL1), preferably human (Accession Number CAG38511.1 or its isoform 1, Accession Number NP_001350527.1 or isoform 2, Accession Number NP_113600.1); GABA Type A receptor associated protein type 2 (GABARAPL2), preferably human (Accession Number CAG47013.1), both of which (GABARAPL1 and GABARAPL2) are referred to herein as Atg8 protein. Related proteins include GABARAP (also referred to as Atg8A) and MAP1LC3A (also referred to as LC3 protein).

The term Syntaxin 16 or (“Stx16”) is used to describe a protein which is a member of the syntaxin or t-SNARE (target-SNAP receptor) family. It is a 307 amino acid protein, the sequence for which can be found at Genbank AAC05647.1 and set forth below. Stx 16 is a SNARE protein involved in trans-Golgi network trafficking. The interaction of Stx 16 may be modulated in certain autophagy-mediated disease state pursuant to the present invention to provide a favorable therapeutic effect on numerous diseases. The inventors have found that modulators of Stx16 can be combined with modulators of Stx17 (both agonists or inhibitors of Stx16/Stx17) to provide particularly effective synergistic therapy for a large number autophagy-mediated disease and/or conditions.

The full human Syntaxin 16 (Stx16) sequence is as follows:
1 matrrltdaf lllrnnsiqn rqllaeqeld eladdrmalv sgisldpeaa igvtkrpppk
61 wvdgvdeiqy dvgrikqkmk elaslhdkhl nrptlddsse eehaieittq eitqlfhrcq
121 ravqpcragk gpapsrrggc lgtwdvaqa lqelstsfih aqsgylkrmk nreersqhff
181 dtsvplmddg ddntlyhrgf tedqlvlveq ntlmveerer eirqivqsis dlneifrdlg
241 amiveqgtvl dridynveqs ciktedglkq lhkaeqyqkk nrkmlvilil fviiivlivv
301 lvgvksr (SEQ ID NO: 1)

The term “Syntaxin 17” or “Stx17” is used to describe an autophagy factor which is phosphorylated by TBK1. The interaction of Stx17 and TBK1 is reflected in phosphorylation of Stx17 by TBK1 and modulation of its function. Surprisingly, these interactions and phosphorylation of Stx17 by TBK1 occur at the earliest stages of autophagy, i.e. at its initiation. The present inventors demonstrate that phosphorylation of Stx17 is important for assembly of the ULK1 complex and that it is critical for autophagy initiation. Pursuant to the present invention, the inventors show that phosphorylated Stx17 and components of the ULK1 complex, localized to the Golgi in the resting state, respond to induction of autophagy by translocating from the Golgi to participate in the formation of mPAS during autophagy initiation. Accordingly, the present invention provides assays and methods for discovering compounds of unknown activity which inhibit phosphorylation of Stx17 and consequently provide activity as inhibitors of autophagy and potential therapeutic agents for the treatment of autophagy diseases and conditions which require phosphorylation of Stx 17 for initiation. Preferably, in the present invention Syntaxin 17 refers to human Syntaxin 17.

The frill human Syntaxin 17 amino acid sequence (Genbank EAW58920.1) is as follows:
1 msedeekvkl rrlepaiqkf ikiviptdle rlrkhqinie kyqrcriwdk lheehinagr
61 tvqqlrsnir eieldclkvr kddlvllkrm idpvkeeasa ataeflqlhl esveelkkqf
121 ndeetllqpp ltrsmtvgga fhtteaeass qsltqiyalp eipqdqnaae swetleadli
181 elsqlvtdfs llvnsqqeki dsiadhvnsa avnveegtkn lgkaakykla alpvagalig
241 gmvggpigll agfkvagiaa algggvlgft ggkliqrkkq kmmekltssc pdlpsqtdkk
301 cs (SEQIDNO:2)

The terms “ATg8 modulator” and “autophagy related protein 8 modulator” refer to direct car indirect modulators (agonists or inhibitors of ATg8) which find particular use in the present invention. ATg8 agonists include agonists (which are principally indirect agonists) and include for example, biguanides such as metformin, phenformin, buformin, proguanil, chlorproguanil and other agents such as bromhexine and its metabolite ambroxol, 20-hydroxyecdysone, a copper or cobalt salt (e.g., cupric sulfate, tribasic copper, copper chelates such as copper glycinate or copper triglycinate, cobalt carbonate, cobalt sulfate, cobalt chloride or cobalt glucoheptanoate) or a mixture thereof.

ATg8 inhibitors include, for example, compounds according to the chemical structure:

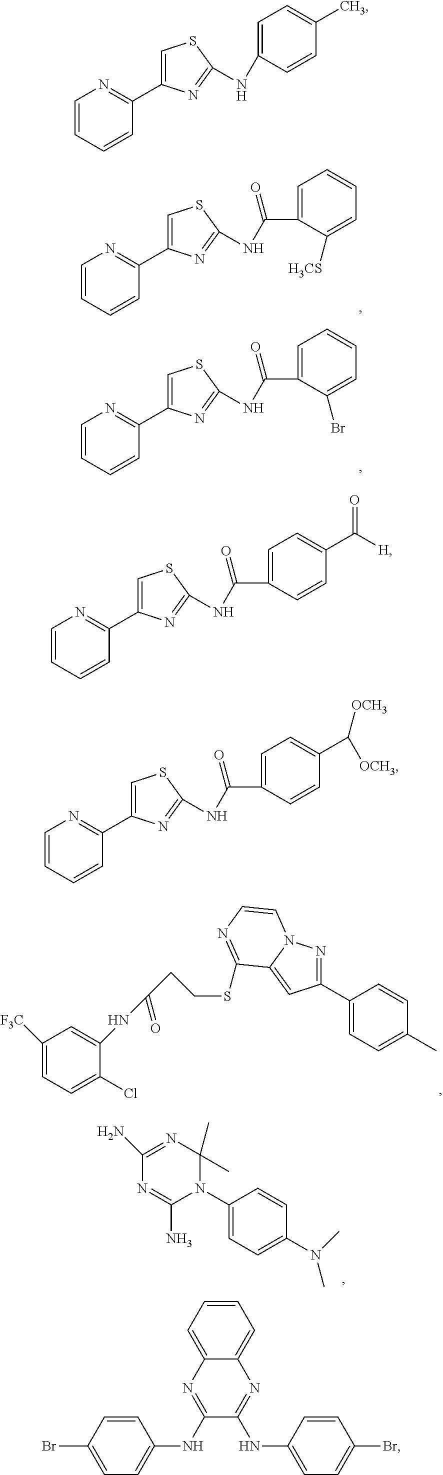

Or a pharmaceutically acceptable salt thereof or a mixture thereof.

In addition, ATg8 inhibitors include TBK1 inhibitors which are indirect inhibitors of ATG8, as well as STX17 inhibitors. These are described herein below. In addition to the TBK1 antagonists (see below), ATg8 antagonists include an anti-ATg8 antibody, which is polyclonal or monoclonal, preferably a monoclonal antibody which is humanized and detects human ATg8. Examples of anti-ATg8 antibodies which may be used in the present invention include humanized anti-ATg8 (Human) mAb which binds to human ATg8 protein.

The terms “Syntaxin 16 modulator” and STX16 modulator refer to direct or indirect modulators (agonists or inhibitors of STX17) which find particular use in file present invention. STX16 agonists include STX16 agonists (which are principally indirect agonists) and include for example, biguanides such as metformin, phenformin, buformin, proguanil, chlorproguanil and other agents such as bromhexine and its metabolite ambroxol.

STX16 antagonists include for example small interfering RNA (siRNA) of the following sequences:

SiRNA for use as STX16 inhibitors have the following sequences:

(SEQ ID NO: 1)
GAACAUGCCAUUGAGAUAA
(SEQ ID NO: 2)
AACCGACGCUUUCUUGUUG
(SEQ ID NO: 3)
GGUGUCAGGCAUCAGCUUA
(SEQ ID NO: 4)
GUAUGAUGUUGGCCGGAUU

In addition to the above STX16 inhibitors, anti-STX16 antibodies, which are polyclonal or monoclonal, preferably a monoclonal antibody which is humanized may be used as an inhibitor of STX16 in the present invention. Examples of anti-Syntaxin 16 antibodies which may be used in the present invention include humanized anti-Syntaxin-16 (Human) mAb which binds to STX16. These antibodies may be delivered into the cell to effect therapy.

The terms “Syntaxin 17 modulator” and “STX17 modulator” refer to direct or indirect modulators (agonists or inhibitors of STX17) which find particular use in the present invention. STX17 agonists for use in the present invention include Dimethhylxanthenone-4-Acetic acid (XAA-5Me): S-Me5thyl-xanthenone-4-Acetic Acid; c-diGMP (cyclic di-GMP) and an Interferon Type I (including IFN-α, IFN-β, IFN-ε IFN-κ, IFN-δ, IFN-τ, IFN-ω and IFN-ν).

STX17 antagonists for use in the present invention include STX17 antagonists (direct or indirect inhibitors, including TBK1 inhibitors) BX795 or MRT67307 (from InvivoGen Corporation), B1-B206, D1-D100, F1-F-160, amlexanox and the compounds listed in columns 249-265 of U.S. Pat. No. 8,569,294, issued Oct. 29, 2013, or a TBK1 inhibitor of US Application Publication No. 2015/0224089, each of which is incorporated by reference in its entirety herein. Note that these TBK1 inhibitors also have an indirect inhibitory effect on ATg8 proteins.

Preferred STX17 (TBK1) Inhibitors from U.S. Pat. No. 8,569,294 include:
2-(3-Phenylpiperazin-1-yl)-3-methyl-6-(4-pyridyl)-3H-pyrimidin-4-one;
2-(3-(4-Fluorophenyl)piperazin-1-yl)-3-methyl-6-(4-pyridyl)-3H-pyrimidin-4-one;
2-(3-(3-Fluorophenyl)piperazin-1-yl)-3-methyl-6-(4-pyridyl)-3H-pyrimidin-4-one;
2-(3-(2-Fluorophenyl)piperazin-1-yl)-3-methyl-6-(4-pyridyl)-3H-pyrimidin-4-one;
2-(3-(4-Chlorophenyl)piperazin-1-yl)-3-methyl-6-(4-pyridyl)-3H-pyrimidin-4-one;
(S)-2-(3-(4-Chlorophenyl piperazin-1-yl)-3-methyl-6-(4-pyridyl)-3H-pyrimidin-4-one;
(R)-2-(3-(4-Chlorophenyl)piperazin-1-yl)-3-methyl-6-(4-pyridyl)-3H-pyrimidin-4-one;
2-(3-(3-Chlorophenyl)piperazin-1-yl)-3-methyl-6-(4-pyridyl)-3H-pyrimidin-4-one;
2-(3-(2-Chlorophenyl)piperazin-1-yl)-3-methyl-6-(4-pyridyl)-3H-pyrimidin-4-one;
2-(3-(4-Bromophenyl)piperazin-1-yl)-3-methyl-6-(4-pyridyl)-3H-pyrimidin-4-one;
2-(3-(3-Bromophenyl)piperazin-1-yl)-3-methyl-6-(4-pyridyl)-3H-pyrimidin-4-one;
2-(3-(2-Bromophenyl)piperazin-1-yl)-3-methyl-6-(4-pyridyl)-3H-pyrimidin-4-one;
2-(3-(4-Methylphenyl)piperazin-1-yl)-3-methyl-6-(4-pyridyl)-3H-pyrimidin-4-one;
2-(3-(3-Methylphenyl)piperazin-1-yl)-3-methyl-6-(4-pyridyl)-3H-pyrimidin-4-one;
2-(3-(2-Methylphenyl)piperazin-1-yl)-3-methyl-6-(4-pyridyl)-3H-pyrimidin-4-one;
2-(3-(4-Cyanophenyl)piperazin-1-yl)-3-methyl-6-(4-pyridyl)-3H-pyrimidin-4-one;
2-(3-(3-Cyanophenyl)piperazin-1-yl)-3-methyl-6-(4-pyridyl)-3H-pyrimidin-4-one;
2-(3-(2-Cyanophenyl)piperazin-1-yl)-3-methyl-6-(4-pyridyl)-3H-pyrimidin-4-one;
2-(3-(4-Methoxyphenylpiperazin-1-yl)-3-methyl-6-(4-pyridyl)-3H-pyrimidin-4-one;
2-(3-(3-Methoxyphenyl)piperazin-1-yl)-3-methyl-6-(4-pyridyl)-3H-pyrimidin-4-one;
2-(3-(2-Methoxyphenyl)piperazin-1-yl)-3-methyl-6-(4-pyridyl)-3H-pyrimidin-4-one;
2-(3-(2-Ethoxyphenyl)piperazin-1-yl)-3-methyl-6-(4-pyridyl)-3H-pyrimidin-4-one;
2-(3-(5-Fluoro-2-methoxyphenyl)piperazin-1-yl)-3-methyl-6-(4-pyridyl)-3H-pyrimidin-4-one;
2-(3-(4-Fluoro-3-methoxyphenyl)piperazin-1-yl)-3-methyl-6-(4-pyridyl)-3H-pyrimidin-4-one;
2-(3-(4-Fluoro-2-methoxyphenyl)piperazin-1-yl)-3-methyl-6-(4-pyridyl)-3H-pyrimidin-4-one;
(S)-2-(3-(4-Fluoro-2-methoxyphenyl)piperazin-1-yl)-3-methyl-6-(4-pyridyl)-3H-pyrimidin-4-one;
(R)-2-(3-(4-Fluoro-2-methoxyphenyl)piperazin-1-yl)-3-methyl-6-(4-pyridyl)-3H-pyrimidin-4-one;
2-(3-(4-Chloro-2-methoxyphenyl)piperazin-1-yl)-3-methyl-6-(4-pyridyl)-3H-pyrimidin-4-one;
2-(3-(4-Fluoro-2-methoxyphenyl)piperazin-1-yl)-3-methyl-6-(4-pyridyl)-3H-pyrimidin-4-one;
2-(3-(2-Fluoro-6-methoxyphenyl)piperazin-1-yl)-3-methyl-6-(4-pyridyl)-3H-pyrimidin-4-one;
2-(3-(5-Bromo-2-methoxyphenyl)piperazin-1-yl)-3-methyl-6-(4-pyridyl)-3H-pyrimidin-4-one;
2-(3-(2-Bromo-4-fluorophenyl)piperazin-1-yl)-3-methyl-6-(4-pyridyl)-3H-pyrimidin-4-one;
2-(3-(2-Chloro-6-fluorophenyl)piperazin-1-yl)-3-methyl-6-(4-pyridyl)-3H-pyrimidin-4-one;
2-(3-(2,4-Difluorophenyl)piperazin-1-yl)-3-methyl-6-(4-pyridyl)-3H-pyrimidin-4-one;
2-(3-(2,6-Difluorophenyl)piperazin-1-yl)-3-methyl-6-(4-pyridyl)-3H-pyrimidin-4-one;
2-(3-(2,6-Dichlorophenyl)piperazin-1-yl)-3-methyl-6-(4-pyridyl)-3H-pyrimidin-4-one;
2-(3-(2,4-Dimethoxyphenyl)piperazin-1-yl)-3-methyl-6-(4-pyridyl)-3H-pyrimidin-4-one;
2-(3-(3,4-Dimethoxyphenyl)piperazin-1-yl)-3-methyl-6-(4-pyridyl)-3H-pyrimidin-4-one;
2-(3-(2,5-Dimethoxyphenyl)piperazin-1-yl)-3-methyl-6-(4-pyridyl)-3H-pyrimidin-4-one;
2-(3-(2,6-Dimethoxyphenyl)piperazin-1-yl)-3-methyl-6-(4-pyridyl)-3H-pyrimidin-4-one;
2-(3-(2,4-Difluoro-6-methoxyphenyl)piperazin-1-yl)-3-methyl-6-(4-pyridyl)-3H-pyrimidin-4-one;
2-(3-(5-Cyano-2-methoxyphenyl)piperazin-1-yl)-3-methyl-6-(4-pyridyl)-3H-pyrimidin-4-one;
2-(3-(4-Cyano-2-methoxyphenyl)piperazin-1-yl)-3-methyl-6-(4-pyridyl)-3H-pyrimidin-4-one;
2-(3-(1-Naphthyl)piperazin-1-yl)-3-methyl-6-(4-pyridyl)-3H-pyrimidin-4-one;
2-(3-(2-Naphthyl)piperazin-1-yl)-3-methyl-6-(4-pyridyl)-3H-pyrimidin-4-one;
2-(3-(2,3-Dihydrobenzofuran-7-yl)piperazin-1-yl)-3-methyl-6-(4-pyridyl)-3H-pyrimidin-4-one;
2-(3-(Benzofuran-2-yl)piperazin-1-yl)-3-methyl-6-(4-pyridyl)-3H-pyrimidin-4-one;
(S)-2-(3-(Benzofuran-2-yl)piperazin-1-yl)-3-methyl-6-(4-pyridyl)-3H-pyrimidin-4-one;
2-(3-(4-(Pyrrolidin-1-yl-methyl)phenyl)piperazin-1-yl)-3-methyl-6-(4-pyridyl)-3H-pyrimidin-4-one;
2-(3-(4-(Pyrrolidin-1-yl)phenyl)piperazin-1-yl)-3-methyl-6-(4-pyridyl)-3H-pyrimidin-4-one;
2-(3-(2-methoxy-4-(pyrrolidin-1-yl)phenyl)piperazin-1-yl)-3-methyl-6-(4-pyridyl)-3H-pyrimidin-4-one;
2-(3-(2-methoxy-5-(pyrrolidin-1-yl)phenyl)piperazin-1-yl)-3-methyl-6-(4-pyridyl)-3H-pyrimidin-4-one;
2-(3-(4-(Phenyl)phenyl)piperazin-1-yl)-3-methyl-6-(4-pyridyl)-3H-pyrimidin-4-one;
2-(3-(4-(4-Fluorophenyl)phenyl)piperazin-1-yl)-3-methyl-6-(4-pyridyl)-3H-pyrimidin-4-one;
2-(3-(4-(4-Methoxyphenyl)phenyl)piperazin-1-yl)-3-methyl-6-(4-pyridyl)-3H-pyrimidin-4-one;
2-(3-(4-(2-Methoxyphenyl)phenyl)piperazin-1-yl)-3-methyl-6-(4-pyridyl)-3H-pyrimidin-4-one;
2-(3-(4-(Morpholin-4-yl)phenyl)piperazin-1-yl)-3-methyl-6-(4-pyridyl)-3H-pyrimidin-4-one;
2-(3-(4-(4-Methylpiperazin-1-yl)phenyl)piperazin-1-yl)-3-methyl-6-(4-pyridyl)-3H-pyrimidin-4-one;
2-(4-Phenylpiperazin-1-yl)-3-methyl-6-(4-pyridyl)-3H-pyrimidin-4-one;
2-(4-Benzylpiperazin-1-yl)-3-methyl-6-(4-pyridyl)-3H-pyrimidin-4-one;
2-(4-Benzoylpiperazin-1-yl)-3-methyl-6-(4-pyridyl)-3H-pyrimidin-4-one;
2-(4-(1,2-Benzisothiazol-3-yl)piperazin-1-yl)-3-methyl-6-(4-pyridyl)-3H-pyrimidin-4-one;
2-(4-Methyl-3-phenylpiperazin-1-yl)-3-methyl-6-(4-pyridyl)-3H-pyrimidin-4-one;
2-(3-(4-Fluoro-2-methoxyphenyl)-4-methylpiperazin-1-yl)-3-methyl-6-(4-pyridyl)-3H-pyrimidin-4-one;
(S)-2-(3-(4-Fluoro-2-methoxyphenyl)-4-methylpiperazin-1-yl)-3H-methyl-6-(4-pyridyl)-3H-pyrimidin-4-one;
(R)-2-(3-(4-Fluoro-2-methoxyphenyl)-4-methylpiperazin-1-yl)-3-methyl-6-(4-pyridyl)-3H-pyrimidin-4-one;
2-(4-Acetyl-3-(4-fluoro-2-methoxyphenyl)piperazin-1-yl)-3-methyl-6-(4-pyridyl)-3H-pyrimidin-4-one;
2-(4-Benzyl-3-(4-fluoro-2-methoxyphenyl)piperazin-1-yl)-3-methyl-6-(4-pyridyl)-3H-pyrimidin-4-one;
2-(4-Benzyl-3-(ethoxycarbonyl)piperazin-1-yl)-3-methyl-6-(4-pyridyl)-3H-pyrimidin-4-one;
2-(4-methyl-3-(1-naphthyl)piperazin-1-yl)-3-methyl-6-(4-pyridyl)-3H-pyrimidin-4-one;
2-(5,5-Dimethyl-3-(2-methoxyphenyl)piperazin-1-yl)-3-methyl-6-(4-pyridyl)-3H-pyrimidin-4-one;
2-(3-Phenylpiperidin-1-yl)-3-methyl-6-(4-pyridyl)-3H-pyrimidin-4-one;
2-(3-(4-Fluorophenyl)piperidin-1-yl)-3-methyl-6-(4-pyridyl)-3H-pyrimidin-4-one;
2-(3-(3-Fluorophenyl)piperidin-1-yl)-3-methyl-6-(4-pyridyl)-3H-pyrimidin-4-one;
2-(3-(2-Fluorophenyl)piperidin-1-yl)-3-methyl-6-(4-pyridyl)-3H-pyrimidin-4-one;
2-(3-(4-Chlorophenyl)piperidin-1-yl)-3-methyl-6-(4-pyridyl)-3H-pyrimidin-4-one;
2-(3-(4-Bromophenyl)piperidin-1-yl)-3-methyl-6-(4-pyridyl)-3H-pyrimidin-4-one;
2-(3-(4-Methoxyphenyl)piperidin-1-yl)-3-methyl-6-(4-pyridyl)-3H-pyrimidin-4-one;
2-(3-(3-Methoxyphenyl)piperidin-1-yl)-3-methyl-6-(4-pyridyl)-3H-pyrimidin-4-one;
2-(3-(2-Methoxyphenyl)piperidin-1-yl)-3-methyl-6-(4-pyridyl)-3H-pyrimidin-4-one;
2-(3-(4-((Pyrrolidin-1-ylmethylphenyl piperidin-1-yl)-3-methyl-6-(4-pyridyl)-3H-pyrimidin-4-one;
(S)-2-(3-(4-(Pyrrolidin-1-yl-methyl)phenyl)piperidin-1-yl)-3-methyl-6-(4-pyridyl)-3H-pyrimidin-4-one;
(R)-2-(3-(4-(Pyrrolidin-1-yl-methyl)phenyl)piperidin-1-yl)-3-methyl-6-(4-pyridyl)-3H-pyrimidin-4-one;
2-(3-Hydroxy-3-phenylpiperidin-1-yl)-3-methyl-6-(4-pyridyl)-3H-pyrimidin-4-one;
2-(3-Phenylpiperazin-1-yl)-3-methyl-6-(4-pyrimidyl)-3H-pyrimidin-4-one;
2-(3-(4-Fluorophenyl)piperazin-1-yl)-3-methyl-6-(4-pyrimidyl)-3H-pyrimidin-4-one;
2-(3-(3-Fluorophenyl)pyrimidin-1-yl)-3-methyl-6-(4-pyrimidyl)-3H-pyrimidin-4-one;
2-(3-(2-Fluorophenyl)piperazin-1-yl)-3-methyl-6-(4-pyrimidyl)-3H-pyrimidin-4-one;
2-(3-(4-Chlorophenyl)piperazin-1-yl)-3-methyl-6-(4-pyrimidyl)-3H-pyrimidin-4-one;
2-(3-(3-Chlorophenyl)piperazin-1-yl)-3-methyl-6-(4-pyrimidyl)-3H-pyrimidin-4-one;
2-(3-(2-Chlorophenyl)piperazin-1-yl)-3-methyl-6-(4-pyrimidyl)-3H-pyrimidin-4-one;
2-(3-(4-Bromophenyl)piperazin-1-yl)-3-methyl-6-(4-pyrimidyl)-3H-pyrimidin-4-one;
2-(3-(2-Bromophenyl)piperazin-1-yl)-3-methyl-6-(4-pyrimidyl)-3H-pyrimidin-4-one;
2-(3-(2-Bromophenyl)piperazin-1-yl)-3-methyl-6-(4-pyrimidyl)-3H-pyrimidin-4-one;
2-(3-(4-Cyanophenyl)piperazin-1-yl)-3-methyl-6-(4-pyrimidyl)-3H-pyrimidin-4-one;
2-(3-(3-Cyanophenyl)piperazin-1-yl)-3-methyl-6-(4-pyrimidyl)-3H-pyrimidin-4-one;
2-(3-(2-Cyanophenyl)piperazin-1-yl)-3-methyl-6-(4-pyrimidyl)-3H-pyrimidin-4-one;
2-(3-(4-Methoxyphenyl)piperazin-1-yl)-3-methyl-6-(4-pyrimidyl)-3H-pyrimidin-4-one;
2-(3-(3 Methoxyphenylpiperazin-1-yl)-3-methyl-6-(4-pyrimidyl)-3H-pyrimidin-4-one;
2-(3-(2-Methoxyphenyl)piperazin-1-yl)-3-methyl-6-(4-pyrimidyl)-3H-pyrimidin-4-one;
2-(3-(2-Ethoxyphenyl)piperazin-1-yl)-3-methyl-6-(4-pyrimidyl)-3H-pyrimidin-4-one;
2-(3-(6-Fluoro-2-methoxyphenyl)piperazin-1-yl)-3-methyl-6-(4-pyrimidyl)-3H-pyrimidin-4-one;
2-(3-(5-Fluoro-2-methoxyphenyl)piperazin-1-yl)-3-methyl-6-(4-pyrimidyl)-3H-pyrimidin-4-one;
2-(3-(4-Fluoro-2-methoxyphenyl)piperazin-1-yl)-3-methyl-6-(4-pyrimidyl)-3H-pyrimidin-4-one;
(S)-2-(3-(4-Fluoro-2-methoxyphenyl)piperazin-1-yl)-3-methyl-6-(4-pyrimidyl)-3H-pyrimidin-4-one;
(R)-2-(3-(4-Fluoro-2-methoxyphenyl)piperazin-1-yl)-3-methyl-6-(4-pyrimidyl)-3H-pyrimidin-4-one;
2-(3-(4-Chloro-2-methoxyphenyl)piperazin-1-yl)-3-methyl-6-(4-pyrimidyl)-3H-pyrimidin-4-one;
2-(3-(5-Bromo-2-methoxyphenyl)piperazin-1-yl)-3-methyl-6-(4-pyrimidyl)-3H-pyrimidin-4-one;
2-(3-(2,6-Dichlorophenyl)piperazin-1-yl)-3-methyl-6-(4-pyridyl)-3H-pyrimidin-4-one;
2-(3-(2,4-Dimethoxyphenyl)piperazin-1-yl)-3-methyl-6-(4-pyridyl)-3H-pyrimidin-4-one;
2-(3-(3,4-Dimethoxyphenyl)piperazin-1-yl)-3-methyl-6-(4-pyridyl)-3H-pyrimidin-4-one;
2-(3-(2,5-Dimethoxyphenyl)piperazin-1-yl)-3-methyl-6-(4-pyrimidyl)-3H-pyrimidin-4-one;
2-(3-(2,6-Dimethoxyphenyl)piperazin-1-yl)-3-methyl-6-(4-pyrimidyl)-3H-pyrimidin-4-one;
2-(3-(2,4-Difluoro-6-Dimethoxyphenyl)piperazin-1-yl)-3-methyl-6-(4-pyrimidyl)-3H-pyrimidin-4-one;
2-(3-(1-Naphthyl)piperazin-1-yl)-3-methyl-6-(4-pyrimidyl)-3H-pyrimidin-4-one;
2-(3-(2-Naphthyl)piperazin-1-yl)-3-methyl-6-(4-pyrimidyl)-3H-pyrimidin-4-one;
2-(3-(2,3-Dihydrobenzofuran-7-yl)piperazin-1-yl)-3-methyl-6-(4-pyrimidyl)-3H-pyrimidin-4-one;
2-(3-(Benzofuran-2-yl piperazin-1-yl)-3-methyl-6-(4-pyrimidyl)-3H-pyrimidin-4-one;
2-(3-(4-(Pyrrolidin-1-yl-methyl)phenyl)piperazin-1-yl)-3-methyl-6-(4-pyrimidyl)-3H-pyrimidin-4-one;
2-(3-(4-(Pyrrolidin-1-yl)phenyl)piperazin-1-yl)-3-methyl-6-(4-pyrimidyl)-3H pyrimidin-4-one;
2-(3-(2-methoxy-4-(pyrrolidin-1-yl)phenyl)piperazin-1-yl)-3-methyl-6-(4-pyrimidyl)-3H-pyrimidin-4-one;
2-(3-(2-methoxy-5-(pyrrolidin-1-yl)phenyl)piperazin-1-yl)-3-methyl-6-(4-pyrimidyl)-3H-pyrimidin-4-one;
2-(3-(4-(Phenyl)phenyl)piperazin-1-yl)-3-methyl-6-(4-pyrimidyl)-3H-pyrimidin-4-one;
2-(3-(4-(4-Fluorophenyl)phenyl)piperazin-1-yl)-3-methyl-6-(4-pyrimidyl)-3H-pyrimidin-4-one;
2-(3-(4-(4-Methoxyphenyl)phenyl)piperazin-1-yl)-3-methyl-6-(4-pyrimidyl)-3H-pyrimidin-4-one;
2-(3-(4-(2-Methoxyphenyl)phenyl)piperazin-1-yl)-3-methyl-6-(4-pyrimidyl)-3H-pyrimidin-4-one;
2-(3-(4-(Morpholin-4-yl)phenyl)piperazin-1-yl)-3-methyl-6-(4-pyrimidyl)-3H-pyrimidin-4-one;
2-(3-(4-(4-Methylpiperazin-1-yl)phenyl)piperazin-1-yl)-3-methyl-6-(4-pyrimidyl)-3H-pyrimidin-4-one;
2-(3-(4-Fluoro-2-methoxyphenyl)-4-methylpiperazin-1-yl)-3-methyl-6-(4-pyrimidyl)-3H-pyrimidin-4-one;
(S)-2-(3-(4-Fluoro-2-methoxyphenyl)-4-methylpiperazin-1-yl)-3-methyl-6-(4-pyrimidyl)-3H-pyrimidin-4-one;
(R)-2-(3-(4-Fluoro-2-methoxyphenyl)-4-methylpiperazin-1-yl)-3-methyl-6-(4-pyrimidyl)-3H-pyrimidin-4-one;
2-(4-Acetyl-3-(4-fluoro-2-methoxyphenyl)piperazin-1-yl)-3-methyl-6-(4-pyrimidyl)-3H-pyrimidin-4-one;
2-(4-Benzyl-3-(4-fluoro-2-methoxyphenyl)piperazin-1-yl)-3-methyl-6-(4-pyrimidyl)-3H-pyrimidin-4-one;
2-(4-(4-fluoro-2-methoxyphenyl)piperidin-1-yl)-3-methyl-6-(4-pyrimidyl)-3H-pyrimidin-4-one;
2-(4-Cyano-4-phenylpiperidin-1-yl)-3-methyl-6-(4-pyrimidyl)-3H-pyrimidin-4-one;
2-(4-(6-Fluorobenofuran-3-yl)piperidin-1-yl)-3-methyl-6-(4-pyrimidyl)-3H-pyrimidin-4-one;
2-(3-(Benzoisoxazol-3-yl)piperidin-1-yl)-3-methyl-6-(4-pyrimidyl)-3H-pyrimidin-4-one;
(S)-2-(3-(Benzoisoxazol-3-yl)piperidin-1-yl)-3-methyl-6-(4-pyrimidyl)-3H-pyrimidin-4-one;
(R)-2-(3-(Benzoisoxazol-3-yl)piperidin-1-yl)-3-methyl-6-(4-pyrimidyl)-3H-pyrimidin-4-one;
2-(3-(6-Fluorobenzoisoxazol-3-yl)piperidin-1-yl)-3-methyl-6-(4-pyrimidyl)-3H-pyrimidin-4-one;
2-(4-(6-Fluorobenzoisoxazol-3-yl)piperidin-1-yl)-3-methyl-6-(4-pyrimidyl)-3H-pyrimidin-4-one;
2-(4-(5-Methylbenzofuran-3-yl)piperidin-1-yl)-3-methyl-6-(4-pyrimidyl)-3H-pyrimidin-4-one; and
2-(4-(6-Fluorobenzothiophene-3-yl)piperidin-1-yl)-3-methyl-6-(4-pyrimidyl)-3H-pyrimidin-4-one.

Additional STX17 (TBK1) inhibitors for use in the present invention include compounds according to the chemical structure:

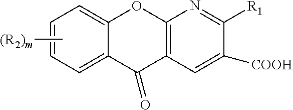

Where R₁ is hydrogen, C₁-C₆ alkyl, phenyl, carboxyl, hydroxyl, O—C₁-C₃ alkyl or amino which is optionally substituted with one C₁-C₃ alkyl group;

m is 0, 1 or 2; and

R₂ is H, C₁-C₆ alkyl, halogen, nitro, hydroxyl, carboxyl, O—C₁-C₃ alkyl, or a butadienylene (—C═C—C═C—) group which forms an unsaturated cyclic ring with any adjacent carbon atoms or nitrogen atom which may be unsubstituted or substituted with at least one C₁-C₃ alkyl group, or

a pharmaceutically acceptable salt thereof.

In alternative embodiments, the inhibitor of Syntaxin 17 is a small molecule compound or antibody which inhibits the phosphorylation of Syntaxin 17. In these embodiments, the preferred inhibitors of Syntaxin 17 phosphorylation include AG1478 (Tyrphostin AG1478) and AG1024 (Tyrphostin AG1024). In embodiments, the inhibitor of Syntaxin 17 is an anti-Syntaxin 17 antibody, which is polyclonal or monoclonal, preferably a monoclonal antibody which is humanized. Examples of anti-Syntaxin 17 antibodies which may be used in the present invention include humanized anti-Syntaxin-17 (Human) mAb which binds to STX17 and prevents phosphorylation of Syntaxin 17. These antibodies may be delivered into the cell to effect therapy.

As used herein, the term “autophagy mediated disease stale or condition” refers to a disease state or condition that results from disruption in autophagy or cellular self-digestion. Autophagy is a cellular pathway involved in protein and organelle degradation, and has a large number of connections to human disease. Autophagic dysfunction which causes disease is associated with metabolic disorders, neurodegeneration, autoimmune diseases, microbial (especially bacterial and viral) infections (especially HIV, HAV, HBV and/or HCV), cancer, aging, cardiovascular diseases and metabolic diseases including diabetes mellitus, among numerous other disease states and/or conditions. Although autophagy plays a principal role as a protective process for fee cell, it also plays a role in cell death. Disease states and/or conditions which are mediated through autophagy (which refers to the feet that fee disease state or condition may manifest itself as a inaction of the increase or decrease in autophagy in the patient or subject to be treated and treatment requires administration of an inhibitor or agonist of autophagy in the patient or subject) include, for example, lysosomal storage diseases (discussed hereinbelow), neurodegeneration (including, for example, Alzheimer's disease, Parkinson's disease, Huntington's disease; other ataxias), immune response (T cell maturation, B cell and T cell homeostasis, counters damaging inflammation), autoimmune diseases and chronic inflammatory diseases resulting in excessive inflammation (these disease states may promote excessive cytokines when autophagy is defective), including, for example, inflammatory bowel disease, including Crohn's disease, rheumatoid arthritis, lupus, multiple sclerosis, chronic obstructive pulmony disease/COPD, pulmonary fibrosis, cystic fibrosis, Sjogren's disease; hyperglycemic disorders, diabetes (I and II), affecting lipid metabolism islet function and/or structure, excessive autophagy may lead to pancreatic β-cell death and related hyperglycemic disorders, including severe insulin resistance, hyperinsulinemia, insulin-resistant diabetes (e.g. Mendenhall's Syndrome, Werner Syndrome, leprechaunism, and lipoatrophic diabetes) and dyslipidemia (e.g. hyperlipidemia as expressed by obese subjects, elevated low-density lipoprotein (LDL), depressed high-density lipoprotein (HDL), and elevated triglycerides) and metabolic syndrome, liver disease (excessive autophagic removal of cellular entities-endoplasmic reticulum), renal disease (apoptosis in plaques, glomerular disease), cardiovascular disease (especially including infarction, ischemia, stroke, pressure overload and complications during reperfusion), muscle degeneration and atrophy, symptoms of aging (including amelioration or the delay in onset or severity or frequency of aging-related symptoms and chronic conditions including muscle atrophy, frailty, metabolic disorders, low grade inflammation, gout, silicosis, atherosclerosis and associated conditions such as cardiac and neurological both central and peripheral manifestations including stroke, age-associated dementia and sporadic form of Alzheimer's disease, and psychiatric conditions including depression), stroke and spinal cord injury, arteriosclerosis, infectious diseases (microbial infections, removes microbes, provides a protective inflammatory response to microbial products, limits adapation of autophagy of host by microbe for enhancement of microbial growth, regulation of innate immunity) including bacterial, fungal, cellular and viral (including secondary disease states or conditions associated wife infectious diseases especially including Mycobacterial infections such as M. tuberculosis, and viral infections such as coronavirus, hepatitis A, B and C and HIV I and II), including AIDS, among numerous others.

In addition, an autophagy disease state or condition includes autoimmune diseases such as myocarditis, Anti-glomercular Base Membrane Nephritis, lupus erythematosus, lupus nephritis, autoimmune hepatitis, primary biliary cirrhosis, alopecia areata, autoimmune urticaria, bullous pemphagoid, dermatitis herpetiformis, epidermolysis bullosa acquisita, linear IgA disease (LAD), pemphigus vulgaris, psoriasis, Addison's disease, autoimmune polyendocrine syndrome I, II and III (APS I, APS II, APS III), autoimmune pancreatitis, type I diabetes, autoimmune thyroiditis, Ord's thyroiditis, Grave's disease, autoimmune oophoritis, Sjogren's syndrome, autoimmune enteropathy, Coeliac disease, Crohn's disease, autoimmune hemolytic anemia, autoimmune lymphoproliferative syndrome, autoimmune neutropenia, autoimmune thrombocytopenic purpura, Cold agglutinin disease, Evans syndrome, pernicious anemia, Adult-onset Still's disease, Felty syndrome, juvenile arthritis, psoriatic arthritis, relapsing polychondritis, rheumatic fever, rheumatoid arthritis, myasthenia gravis, acute disseminated encephalomyelitis (ADEM), balo concentric sclerosis, Guillain-Barré syndrome, Hashimoto's encephalopathy, chronic inflammatory demyelinating polyneuropathy, Lambert-Eaton myasthenic syndrome, multiple sclerosis, autoimmune uveitis, Graves opthalmopathy, Granulomatosis with polyangitis (GPA), Kawasaki's disease, vasculitis and chronic fatigue syndrome, among others.

As used herein, the term “autophagy mediated disease state or condition” refers to a disease state or condition that results from disruption in autophagy or cellular self-digestion. Autophagy is a cellular pathway involved in protein and organelle degradation, and has a large number of connections to human disease. Autophagic dysfunction is associated with cancer, neurodegeneration, microbial infection and ageing, among numerous other disease states and/or conditions. Although autophagy plays a principal role as a protective process for the cell, it also plays a role in cell death. Disease states and/or conditions which are mediated through autophagy (which refers to the fact that the disease state or condition may manifest itself as a function of the increase or decrease in autophagy in the patient or subject to be treated and treatment requires administration of an inhibitor or agonist of autophagy in the patient or subject) include, for example, cancer, including metastasis of cancer, lysosomal storage diseases (discussed hereinbelow), neurodegeneration (including, for example, Alzheimer's disease, Parkinson's disease, Huntington's disease; other ataxias), immune response (T cell maturation, B cell and T cell homeostasis, counters damaging inflammation) and chronic inflammatory diseases (may promote excessive cytokines when autophagy is defective), including, for example, inflammatory bowel disease, including Crohn's disease, rheumatoid arthritis, lupus, multiple sclerosis, chronic obstructive pulmony disease/COPD, pulmonary fibrosis, cystic fibrosis, Sjogren's disease; hyperglycemic disorders, diabetes (T and B), affecting lipid metabolism islet function and/or structure, excessive autophagy may lead to pancreatic β-cell death and related hyperglycemic disorders, including severe insulin resistance, hyperinsulinemia, insulin-resistant diabetes (e.g. Mendenhall's Syndrome, Werner Syndrome, leprechaunism, and lipoatrophic diabetes) and dyslipidemia (e.g. hyperlipidemia as expressed by obese subjects, elevated low-density lipoprotein (LDL), depressed high-density lipoprotein (HDL), and elevated triglycerides) and metabolic syndrome, liver disease (excessive autophagic removal of cellular entities-endoplasmic reticulum), renal disease (apoptosis in plaques, glomerular disease), cardiovascular disease (especially including ischemia, stroke, pressure overload and complications during reperfusion), muscle degeneration and atrophy, symptoms of aging (including amelioration or the delay in onset or severity or frequency of aging-related symptoms and chronic conditions including muscle atrophy, frailty, metabolic disorders, low grade inflammation, atherosclerosis and associated conditions such as cardiac and neurological both central and peripheral manifestations including stroke, age-associated dementia and sporadic form of Alzheimer's disease, pre-cancerous states, and psychiatric conditions including depression), stroke and spinal cord injury, arteriosclerosis, infectious diseases (microbial infections, removes microbes, provides a protective inflammatory response to microbial products, limits adapation of autophagy of host by microbe for enhancement of microbial growth, regulation of innate immunity) including bacterial, fungal, cellular and viral (including secondary disease states or conditions associated with infectious diseases), including AIDS and tuberculosis, among others, development (including erythrocyte differentiation), embryogenesis/fertility/infertility (embryo implantation and neonate survival after termination of transplacental supply of nutrients, removal of dead cells during programmed cell death) and ageing (increased autophagy leads to the removal of damaged organelles or aggregated macromolecules to increase health and prolong lire, but increased levels of autophagy in children/young adults may lead to muscle and organ wasting resulting in ageing/progeria).

The term “lysosomal storage disorder” refers to a disease state or condition that results from a defect in lysosomomal storage. These disease states or conditions generally occur when the lysosome malfunctions. Lysosomal storage disorders are caused by lysosomal dysfunction usually as a consequence of deficiency of a single enzyme required for the metabolism of lipids, glycoproteins or mucopolysaccharides. The incidence of lysosomal storage disorder (collectively) occurs at an incidence of about about 1:5,000-1:10,000. The lysosome is commonly referred to as the cell's recycling center because it processes unwanted material into substances that the cell can utilize. Lysosomes break down this unwanted matter via high specialized enzymes. Lysosomal disorders generally are triggered when a particular enzyme exists in too small an amount or is missing altogether. When this happens, substances accumulate in the cell. In other words, when the lysosome doesn't function normally, excess products destined for breakdown and recycling are stored in the cell. Lysosomal storage disorders are genetic diseases, but these may be treated using autophagy modulators (autostatins) as described herein. All of these diseases share a common biochemical characteristic, i.e., that all lysosomal disorders originate from an abnormal accumulation of substances inside the lysosome. Lysosomal storage diseases mostly affect children who often die as a consequence at an early stage of life, many within a few months or years of birth. Many other children die of this disease following years of suffering from various symptoms of their particular disorder.

Examples of lysosomal storage diseases include, for example, activator deficiency/GM2 gangliosidosis, alpha-mannosidosis, aspartylflucoaminuria, cholesteryl ester storage disease, chronic hexosaminidase A deficiency, cystinosis, Danon disease, Fabry disease, Farber disease, fucosidosis, galactosialidosis, Gaucher Disease (Types I, II and III), GM? Ganliosidosis, including infantile, late infantile/juvenile and adult/chronic), Hunter syndrome (MPS II), I-Cell disease/Mucolipidosis n, Infantile Free Sialic Acid Storage Disease (ISSD), Juvenile Hexosaminidase A Deficiency, Krabbe disease, Lysosomal acid lipase deficiency, Metachromatic Leukodystrophy, Hurler syndrome, Scheie syndrome, Hurler-Scheie syndrome, Sanfilippo syndrome, Morquio Type A and B, Maroteaux-Lamy, Sly syndrome, mucolipidosis, multiple sulfate deficiency, Niemann-Pick disease, Neuronal ceroid lipofuscinoses, CLN6 disease, Jansky-Bielschowsky disease, Pompe disease, pycnodysostosis, Sandhoff disease, Schindler disease, Tay-Sachs and Wolman disease, among others.

An “inflammation-associated metabolic disorder” includes, but is not limited to, lung diseases, hyperglycemic disorders including diabetes and disorders resulting from insulin resistance, such as Type I and Type II diabetes, as well as severe insulin resistance, hyperinsulinemia, and dyslipidemia or a lipid-related metabolic disorder (e.g. hyperlipidemia (e.g., as expressed by obese subjects), elevated low-density lipoprotein (LDL), depressed high-density lipoprotein (HDL), and elevated triglycerides) and insulin-resistant diabetes, such as Mendenhall's Syndrome, Werner Syndrome, leprechaunism, and lipoatrophic diabetes, renal disorders, such as acute and chronic renal insufficiency, end-stage chronic renal failure, glomerulonephritis, interstitial nephritis, pyelonephritis, glomerulosclerosis, e.g., Kimmelstiel-Wilson in diabetic patients and kidney failure after kidney transplantation, obesity, GH-deficiency, GH resistance, Turner's syndrome, Laron's syndrome, short stature, increased fat mass-to-lean ratios, immunodeficiencies including decreased CD4⁺ T cell counts and decreased immune tolerance or chemotherapy-induced tissue damage, bone marrow transplantation, diseases or insufficiencies of cardiac structure or function such as heart dysfunctions and congestive heart failure, neuronal, neurological, or neuromuscular disorders, e.g., diseases of the central nervous system including Alzheimer's disease, or Parkinson's disease or multiple sclerosis, and diseases of the peripheral nervous system and musculature including peripheral neuropathy, muscular dystrophy, or myotonic dystrophy, and catabolic states, including those associated with wasting caused by any condition, including, e.g. mental health condition (e.g., anorexia nervosa), trauma or wounding or infection such as with a bacterium or human virus such as HIV, wounds, skin disorders, gut structure and function that need restoration, and so forth.

An “inflammation-associated metabolic disorder” also includes a cancer and an “infectious disease” as defined herein, as well as disorders of bone or cartilage growth in children, including short stature, and in children and adults disorders of cartilage and bone in children and adults, including arthritis and osteoporosis. An “inflammation-associated metabolic disorder” includes a combination of two or more of the above disorders (e.g., osteoporosis that is a sequela of a catabolic state). Specific disorders of particular interest targeted for treatment herein are diabetes and obesity, heart dysfunctions, kidney disorders, neurological disorders, bone disorders, whole body growth disorders, and immunological disorders.

In one embodiment, “inflammation-associated metabolic disorder” includes: central obesity, dyslipidemia including particularly hypertriglyceridemia, low HDL cholesterol, small dense LDL particles and postpranial lipemia; glucose intolerance such as impaired fasting glucose; insulin resistance and hypertension, and diabetes. The term “diabetes” is used to describe diabetes mellitus type I or type II. The present invention relates to a method for improving renal function and symptoms, conditions and disease states which occur secondary to impaired renal function in patients or subjects with diabetes as otherwise described herein. It is noted that in diabetes mellitus type I and II, renal function is impaired from collagen deposits, and not from cysts in the other disease states treated by the present invention.

Mycobacterial infections often manifest as diseases such as tuberculosis. Human infections caused by mycobacteria have been widespread since ancient times, and tuberculosis remains a leading cause of death today. Although the incidence of the disease declined, in parallel with advancing standards of living, since the mid-nineteenth century, mycobacterial diseases still constitute a leading cause of morbidity and mortality in countries with limited medical resources. Additionally, mycobacterial diseases can cause overwhelming, disseminated disease in immunocompromised patients, hi spite of the efforts of numerous health organizations worldwide, the eradication of mycobacterial diseases has never been achieved, nor is eradication imminent Nearly one third of the world's population is infected with Mycobacterium tuberculosis complex, commonly referred to as tuberculosis (TB), with approximately 8 million new cases, and two to three million deaths attributable to TB yearly. Tuberculosis (TB) is the cause of the largest number of human deaths attributable to a single etiologic agent (see Dye et al., J. Am. Med. Association, 282, 677-686, (1999); and 2000 WHO/OMS Press Release).

Mycobacteria other than M. tuberculosis are increasingly found in opportunistic infections that plague the AIDS patient Organisms from the M. avium-intracellulare complex (MAC), especially serotypes four and eight, account for 68% of the mycobacterial isolates from AIDS patients. Enormous numbers of MAC are found (up to 10¹⁰ acid-fast bacilli per gram of tissue), and consequently, the prognosis for the infected AIDS patient is poor.

In many countries the only measure for TB control has been vaccination with M. bovis bacille Calmette-Guerin (BCG). The overall vaccine efficacy of BCG against TB, however, is about 50% with extreme variations ranging from 0% to 80% between different field trials. The widespread emergence of multiple drug-resistant M. tuberculosis strains is also a concern.

M. tuberculosis belongs to the group of intracellular bacteria that replicate within the phagosomal vacuoles of resting macrophages, thus protection against TB depends on T cell-mediated immunity. Several studies in mice and humans, however, have shown that Mycobacteria stimulate antigen-specific, major histocompatibility complex (MHC) class II- or class I-restricted CD4 and CD8 T cells, respectively. The important role of MHC class I-restricted CD8 T cells was convincingly demonstrated by the failure of β2-microglobulin) deficient mice to control experimental M. tuberculosis infection.

As used herein, the term “tuberculosis” comprises disease states usually associated with infectious caused by mycobacteria species comprising M. tuberculosis complex. The term “tuberculosis” is also associated with mycobacterial infections caused by mycobacteria other than M. tuberculosis. Other mycobacterial species include M. avium-intracellulare, M. kansarii, M. fortutium, M. chelonae, M. leprae, M. africanum, and M. microti, M. avium paratuberculosis, M. intracellulare, M. scrofulaceum, M. xenopi, M. marinum, M. ulcerans.

An “infectious disease” includes but is limited to those caused by bacterial, mycological, parasitic, and viral agents. Examples of such infectious agents include the following: staphylococcus, streptococcaceae, neisseriaaceae, cocci, enterobacteriaceae, pseudomonadaceae, vibrionaceae, Campylobacter, pasteurellaceae, bordetella, francisella, brucella, legionellaceae, bacteroidaceae, gram-negative bacilli, clostridium, corynebacterium, propionibacterium, gram-positive bacilli, anthrax, actinomyces, nocardia, mycobacterium, treponema, borrelia, leptospira, mycoplasma, ureaplasma, rickettsia, chlamydiae, systemic mycoses, opportunistic mycoses, protozoa, nematodes, trematodes, cestodes, adenoviruses, herpesviruses, poxviruses, papovaviruses, hepatitis viruses, orthomyxoviruses, paramyxoviruses, coronaviruses, picornaviruses, reoviruses, togaviruses, flaviviruses, bunyaviridae, rhabdoviruses, coronavirus, human immunodeficiency virus and retroviruses, hi embodiments of the invention, it has been discovered that coronavirus is susceptible to STX17 agonists as otherwise described herein, especially when combined with STX16 agonists and optionally ATg8 agonists.

In certain embodiments, an “infectious disease” is selected from the group consisting of tuberculosis, leprosy, Crohn's Disease, acquired immunodeficiency syndrome, Lyme disease, cat-scratch disease, Rocky Mountain spotted fever and influenza or a viral infection selected from HIV (I and/or II), hepatitis B virus (HBV) or hepatitis C virus (HCV).

The term “mitophagy” refers to an autophagy-mediated disease state or condition where the patient evidences mitochondrial dysfunction. These disease states include, for example, hepatic encephalopathy, liver toxicity (heavy metals, especially cadmium), Alzheimer's disease, Parkinson's disease, mild cognitive impairment, autism, mitochondrial lymphoblast dysfunction, mitochondrial fibroblast dysfunction, mitochondrial neuronal dysfunction, mitochondrial cardiac dysfunction, cardiac hypertrophy and mitochondrial adrenocortical dysfunction, among numerous others.

The term “pexophagy” refers to an autophagy-mediated disease state or condition where the patient evidences peroxisome dysfunction. Disease states and/or conditions related to pexophagy include Zellweger syndrome, rhizomelic chondrodysplasia punctata and infantile refsum disease, among others.

The term “ribophagy” refers to an autophagy-mediated disease state or condition where the patient evidences dysfunction in the balance of ribosome biogenesis and the breakdown of ribosomes. Disease states and/or conditions related to ribophagy include Tuberculosis, Alzheimer's disease, gastric cancer, Diamond-Blackfan anemia, Treacher-Collins Syndrome (TCS), Native American Indian childhood Cirrhosis (NAICC), male infertility, Bowen-Conradi syndrome (BCS), alopecia-neurological defects-endocrinopathy syndrome (AWE syndrome), Schwachman-Diamond Syndrome, primary open angle glaucoma (POAG), neurofibromatosis type 1 (NFI), sever macrocytic anemia, myelodysplastic syndrome and predisposition to cancer.

The term “xenophagy” refers to an autophagy-mediated disease state or condition where the patient evidences dysfunction associated with infectious, including Mycoplasma infections such as M. tuberculosis.

The term “autophagy modulator agent” or “additional autophagy modulator” is used to describe an optional agent which is used in the compositions and/or methods according to the present invention in order to enhance or inhibit an autophagy response in an autophagy mediated disease state which is otherwise treated, ameliorated, inhibited and/or resolved by another agent other than a ATG8 inhibitor or agonist and/or a STX16 and/or STX17 inhibitor or agonist such as for example, Galectin-8 and/or Galectin-9, a modulator/upregulator of Galectin-8 and/or Galectin-9, or an agent which acts similar to Galectin-8 as an inhibitor of mTOR and/or Galectin-9 as a modulator (upregulator) of AMPKinase or a mixture thereof, optionally in combination with a lysosomotropic agent. Additional autophagy modulators include, but are not limited to, autophagy agonists (such as flubendazole, hexachlorophane, propidium iodide, bepridil, clomiphene citrate (Z,E), GBR 12909, propafenone, metixene, dipivefrin, fluvoxamine, dicyclomine, dimethisoquin, ticlopidine, memantine, bromhexine, ambroxol, norcyclobenzaprine, diperodon, nortriptyline or a mixture thereof or their pharmaceutically acceptable salts). Additional autophagy modulators which may be used in the present invention to inhibit, prevent and/or treat an autophagy mediated disease state and/or condition include one or more of benzethonium, niclosamide, monensin, bromperidol, levobunolol, dehydroisoandosterone 3-acetate, sertraline, tamoxifen, reserpine, hexachlorophene, dipyridamole, harmaline, prazosin, lidoflazine, thiethylperazine, dextromethorphan, desipramine, mebendazole, canrenone, chlorprothixene, maprotiline, homochlorcyclizine, loperamide, nicardipine, dexfenfluramine, nilvadipine, dosulepin, biperiden, denatonium, etomidate, toremifene, tomoxetine, clorgyline, zotepine, beta-escin, tridihexethyl, ceftazidime, methoxy-6-harmalan, melengestrol, albendazole, rimantadine, chlorpromazine, pergolide, cloperastine, prednicarbate, haloperidol, clotrimazole, nitrofural, iopanoic acid, naftopidil, Methimazole, Trimeprazine, Ethoxyquin, Clocortolone, Doxycycline, Pirlindole mesylate, Doxazosin, Deptropine, Nocodazole, Scopolamine, Oxybenzone, Halcinonide, Oxybutynin, Miconazole, Clomipramine, Cyproheptadine, Doxepin, Dyclonine, Salbutamol, Flavoxate, Amoxapine, Fenofibrate, Pimethixene and mixtures thereof. The autophagy modulator may be included as optional agents in compositions according to the present invention or used in conjugation with therapies as otherwise described herein to treat an autophagy mediated disease state or condition.

The term “co-administration” or “combination therapy” is used to describe a therapy in which at least two active compounds in effective amounts are used to treat an autophagy mediated disease state or condition as otherwise described herein, either at the same time or within dosing or administration schedules defined further herein or ascertainable by those of ordinary skill in the art. Although the term co-administration preferably includes the administration of two active compounds to the patient at the same time, it is not necessary that the compounds be administered to the patient at the same time, although effective amounts of the individual compounds will be present in the patient at the same time. In addition, in certain embodiments, co-administration will refer to the feet that two compounds are administered at significantly different times, but the effects of the two compounds are present at the same time. Thus, the term co-administration includes an administration in which one active agent is administered at approximately the same time (contemporaneously), or from about one to several minutes to about 24 hours or more after or before the other active agent is administered.

In yet additional embodiments, additional bioactive agents may be further included in compositions according to the present invention in combination with agents which control mTor response to endomembrane damage (e.g. Galectin-8 and/or Galectin-9, a modulator/upregulator of Galectin-8 and/or Galectin-9, or an agent which acts similar to Galectin-8 as an inhibitor of mTOR and/or Galectin-9 as a modulator (upregulator) of AMPKinase or a mixture thereof which may optionally be combined with a lysosomotropic agent and/or an autophagy modulator) and may be any bioactive agent such as an additional mTOR inhibitor (i.e., other than Galectin-8) such as Dactolisib (BEZ23S, NVP-BEX235, rapamycin, everolimis, AZD8055, Temsilolimus, PI-103, KU0063794, Torkinib (PP242), tacrolimus (FK506), ridaforolimus (deforolimus, MK-8669), Sapanisertib (INK 128), Voxtalisib (XL765), Torin 1, Torin 2, Omipalisib (GSK458), OSI-027, PF-04691502, Apitolisib (RG7422), GSK1059615, Gadatolisib (PKI-587), WYE-354, Vistusertib (AZD2014), WYE-132, BGT226, Palomid 529 (P529), PP121, WYE-687, WAY-600, ETP-46464, GDC-0349, XL-388, CC-115, Zotarolimus (ABT-578), GDC-0084, CZ415, 3DBO, SF2523, MHY1485, Chrysophanic Acid, CC-223, LY3023414, among others, with Torin1, Torin 2, pp242, rapamycin/serolimus (which also may function as an autophagy modulator), everolimus, temsirolomis, ridaforolimis, zotarolimis, 32-dexoy-rapamycin, among others being preferred. mTorr inhibitors also include for example, epigallocatechin gallate (EGCG), caffeine, curcumin or reseveratrol (which mTOR inhibitors find particular use as enhancers of autophagy using the compounds disclosed herein), hi certain embodiments, an additional mTOR inhibitor as described above, or more often selected from the group consisting of Torin, pp242, rapamycin/serolimus, everolimus, temsirolomis, ridaforolimis, zotarolimis, 32-dexoy-rapamycin, epigallocatechin gallate (EGCG), caffeine, curcumin or reseveratrol and mixtures thereof may be combined with at least one agent selected from the group consisting of digoxin, xylazine, hexetidine and sertindole, the combination of such agents being effective as autophagy modulators in combination.

The terms “cancer” and “neoplasia” are used throughout the specification to refer to the pathological process that results in the formation and growth of a cancerous or malignant neoplasm, i.e., abnormal tissue that grows by cellular proliferation, often more rapidly than normal and continues to grow after the stimuli that initiated the new growth cease. Malignant neoplasms show partial or complete lack of structural organization and functional coordination with the normal tissue and most invade surrounding tissues, metastasize to several sites, and are likely to recur after attempted removal and to cause the death of the patient unless adequately treated.

As used herein, the terms malignant neoplasia and cancer are used synonymously to describe all cancerous disease states and embraces or encompasses the pathological process associated with malignant hematogenous, ascitic and solid tumors. Representative cancers include, for example, stomach, colon, rectal, liver, pancreatic, lung, breast, cervix uteri, corpus uteri, ovary, prostate, testis, bladder, renal, brain/CNS, head and neck, throat, Hodgkin's disease, non-Hodgkin's lymphoma, multiple myeloma, leukemia, melanoma, non-melanoma skin cancer (especially basal cell carcinoma or squamous cell carcinoma), acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma, among others, which may be treated by one or more compounds according to the present invention. In certain aspects, the cancer which is treated is lung cancer, breast cancer, ovarian cancer and/or prostate cancer.

Neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms (cancer) are distinguished from benign neoplasms in that the former show a greater degree of anaplasia, or loss of differentiation and orientation of cells, and have the properties of invasion and metastasis. Examples of neoplasms or neoplasias from which the target cell of the present invention may be derived include, without limitation, carcinomas (e.g., squamous-cell carcinomas, adenocarcinomas, hepatocellular carcinomas, and renal cell carcinomas), particularly diose of the bladder, bowel, breast, cervix, colon, esophagus, head, kidney, liver, long, neck, ovary, pancreas, prostate, stomach and thyroid; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas (Beers and Berkow (eds.). The Merck Manual of Diagnosis and Therapy, 17.sup.th ed. (Whitehouse Station, N.J.: Merck Research Laboratories, 1999) 973-74, 976, 986, 988, 991). All of these neoplasms may be treated using compounds according to the present invention.

Representative common cancers to be treated with compounds according to the present invention include, for example, prostate cancer, metastatic prostate cancer, stomach, colon, rectal, liver, pancreatic, lung, breast, cervix uteri, corpus uteri, ovary, testis, bladder, renal, brain/CNS, head and neck, throat, Hodgkin's disease, non-Hodgkin's lymphoma, multiple myeloma, leukemia, melanoma, non-melanoma skin cancer, acute lymphocytic leukemia, acute myelogenous leukemia, Ewing's sarcoma, small cell lung cancer, choriocarcinoma, rhabdomyosarcoma, Wilms' tumor, neuroblastoma, hairy cell leukemia, mouth/pharynx, oesophagus, larynx, kidney cancer and lymphoma, among others, which may be treated by one or more compounds according to the present invention. Because of the activity of the present compounds, the present invention has general applicability treating virtually any cancer in any tissue, thus the compounds, compositions and methods of the present invention are generally applicable to the treatment of cancer and in reducing the likelihood of development of cancer and/or the metastasis of an existing cancer.

In certain particular aspects of the present invention, the cancer which is treated is metastatic cancer, a recurrent cancer or a drug resistant cancer, especially including a drug resistant cancer. Separately, metastatic cancer may be found in virtually all tissues of a cancer patient in late stages of the disease, typically metastatic cancer is found in lymph system/nodes (lymphoma), in bones, in lungs, in bladder tissue, in kidney tissue, liver tissue and in virtually any tissue, including brain (brain cancer/tumor). Thus, the present invention is generally applicable and may be used to treat any cancer in any tissue, regardless of etiology.

The term “tumor” is used to describe a malignant or benign growth or tumefacent.

The term “additional anti-cancer compound”, “additional anti-cancer drug” or “additional anti-cancer agent” is used to describe any compound (including its derivatives) which may be used to treat cancer. The “additional anti-cancer compound”, “additional anti-cancer drug” or “additional anti-cancer agent” can be an anticancer agent which is distinguishable from a CIAE-inducing anticancer ingredient such as a taxane, vinca alkaloid and/or radiation sensitizing agent otherwise used as chemotherapy/cancer therapy agents herein. In many instances, the co-administration of another anti-cancer compound according to the present invention results in a synergistic anti-cancer effect. Exemplary anti-cancer compounds for co-administration with formulations according to the present invention include anti-metabolites agents which are broadly characterized as antimetabolites, inhibitors of topoisomerase I and II, alkylating agents and microtubule inhibitors (e.g., taxol), as well as tyrosine kinase inhibitors (e.g., surafenib), EGF kinase inhibitors (e.g., tarceva or erlotinib) and tyrosine kinase inhibitors or ABL kinase inhibitors (e.g. imatinib).

Anti-cancer compounds for co-administration include, for example, agent(s) which may be co-administered with compounds according to the present invention in the treatment of cancer. These agents include chemotherapeutic agents and include one or more members selected from the group consisting of everolimus, trabectedin, abraxane, TLK 286, AV-299, DN-101, pazopanib, GSK690693, RTA 744, ON 0910.Na, AZO 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PDC-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (rack) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid,N-4-[2-(2-amino-4,7-dihydro-4-oxo-1H-pyrrolo[2,3-d 3pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258,); 3-[5-(methylsulfonylpiperadinemethyl)-indolyl]-quinolone, vatalanib, AG-013736, AVE-0005, the acetate salt of [D-Ser(Bu t) 6,Azgly 10] (pyro-Glu-His-Tip-Ser-Tyr-D-Ser(Bu t)-Leu-Arg-Pro-Azgly-NH₂ acetate [C₅₉H₈₄N₁₈Oi₄-(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethyistilbestrol, epirubicin, fludarabine, fludrocortisone, fluoxymesterone, flutamide, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, Vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-fiee paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolinuis, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendranate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitaut, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa, ipilimumab, nivolomuab, pembrolizumab, dabrafenib, trametinib and vemurafenib among others.

Co-administration of one of the formulations of the invention with another anticancer agent will often result in a synergistic enhancement of the anticancer activity of the other anticancer agent, an unexpected result. One or more of the present formulations comprising a ATg8 modulator and/or a STX16 modulator and/or a STX17 modulator optionally in combination with an autophagy modulator (autostatin) as described herein may also be co-administered with another bioactive agent (e.g., antiviral agent, antihyperproliferative disease agent, agents which treat chronic inflammatory disease, among others as otherwise described herein).

The term “antiviral agent” refers to an agent which may be used in combination with autophagy modulators (autostatins) as otherwise described herein to treat viral infections, especially including HIV infections, HBV infections and/or HCV infections. Exemplary anti-HIV agents include, for example, nucleoside reverse transcriptase inhibitors (NRTI), non-nucleoside reverse transcriptase inhibitors (NNRTI), protease inhibitors, fusion inhibitors, among others, exemplary compounds of which may include, for example, 3TC (Lamivudine), AZT (Zidovudine), (−)-FTC, ddI (Didanosine), ddC (zalcitabine), abacavir (ABC), tenofovir (PMPA), D-D4FC (Reverset), D4T (Stavudine), Racivir, L-FddC, L-FD4C, NVP (Nevirapine), DLV (Delavirdine), EFV (Efavirenz), SQVM (Saquinavir mesylate), RTV (Ritonavir), IDV (Indinavir), SQV (Saquinavir), NFV (Nelfinavir), APV (Anprenavir), LPV (Lopinavir), fusion inhibitors such as T20, among others, fuseon and mixtures thereof including anti-HIV compounds presently in clinical trials or in development. Exemplary anti-HBV agents include, for example, hepsera (adefovir dipivoxil), lamivudine, entecavir, telbivudine, tenofovir, emtricitabine, clevudine, valtoricitabine, amdoxovir, pradefovir, racivir, BAM 205, nitazoxanide, UT 231-B, Bay 41-4109, EHT899, zadaxin (thymosin alpha-1) and mixtures thereof. Anti-HCV agents include, for example, interferon, pegylated intergeron, ribavirin, NM 283, VX-950 (telaprevir), SCH 50304, TMC435, VX-500, BX-813, SCH503034, R1626, ITMN-191 (R7227), R7128, PF-868554, TT033, CGH-759, GI 5005, MK-7009, SIRNA-034, MK-0608, A-837093, GS 9190, ACH-1095, GSK625433, TG4040 (MVA-HCV), A-831, F351, NS5A, NS4B, ANA598, A-689, GNI-104, IDX102, ADX184, GL59728, GL60667, PSI-7851, TLR9 Agonist, PHX1766, SP-30 and mixtures thereof.

The term “anti-mycobacterial agent” or “anti-tuberculosis agent” shall refer to traditional agents which are used in the art for the treatment of mycobacterial infections, especially including tuberculosis agents. These agents include, for example, one or more of aminosalicyclic acid/aminosalicylate sodium, capreomycin sulfate, clofazimine, cycloserine, ethambutol hydrochloride (myambutol), kanamycin sulfate, pyrazinamide, rifabutin, rifampin, rifapentine, streptomycin sulfate, gatifloxacin and mixtures thereof, all in therapeutically effective amounts, which may be used in conjunction with other agents described herein in the treatment of mycobacterial infections, especially including Mycobacterium tuberculosis (“tuberculosis”) infections.

According to various embodiments, the combination of compositions and/or compounds according to the present invention may be used for treatment or prevention purposes in the form of a pharmaceutical composition. This pharmaceutical composition may comprise one or more of an active ingredient as described herein.

As indicated, the pharmaceutical composition may also comprise a pharmaceutically acceptable excipient, additive or inert carrier. The pharmaceutically acceptable excipient, additive or inert carrier may be in a form chosen from a solid, semi-solid, and liquid. The pharmaceutically acceptable excipient or additive may be chosen from a starch, crystalline cellulose, sodium starch glycolate, polyvinylpyrolidone, polyvinylpolypyrolidone, sodium acetate, magnesium stearate, sodium laurylsulfate, sucrose, gelatin, silicic acid, polyethylene glycol, water, alcohol, propylene glycol, vegetable oil, corn oil, peanut oil, olive oil, surfactants, lubricants, disintegrating agents, preservative agents, flavoring agents, pigments, and other conventional additives. The pharmaceutical composition may be formulated by admixing the active with a pharmaceutically acceptable excipient or additive.

The pharmaceutical composition may be in a form chosen from sterile isotonic aqueous solutions, pulls, drops, pastes, cream, quay (including aerosols), capsules, tablets, sugar coating tablets, granules, suppositories, liquid, lotion, suspension, emulsion, ointment, gel, and the like. Administration route may be chosen from subcutaneous, intravenous, intrathecal, intestinal, parenteral, oral, buccal, nasal, intramuscular, transcutaneous, transdermal, intranasal, intraperitoneal, and topical. The pharmaceutical compositions may be immediate release, sustained/controlled release, or a combination of immediate release and sustained/controlled release depending upon the compound(s) to be delivered, the compound(s), if any, to be coadministered, as well as the disease state and/or condition to be treated with the pharmaceutical composition. A pharmaceutical composition may be formulated with differing compartments or layers in order to facilitate effective administration of any variety consistent with good pharmaceutical practice.

The subject or patient may be chosen from, for example, a human, a mammal such as domesticated animal, or other animal. The subject may have one or more of the disease states, conditions or symptoms associated with autophagy as otherwise described herein.

The compounds according to the present invention may be administered in an effective amount to treat or reduce the likelihood of an autophagy-mediated disease and/or condition as well one or more symptoms associated with the disease state or condition. One of ordinary skill in the art would be readily able to determine an effective amount of active ingredient by taking into consideration several variables including, but not limited to, the animal subject, age, sex, weight, site of the disease state or condition in the patient, previous medical history, other medications, etc.

For example, the dose of an active ingredient which is useful in the treatment of an autophagy mediated disease state, condition and/or symptom for a human patient is that which is an effective amount and may range from as little as 100 μg or even less to at least about 1,000 mg or more, often 1 mg, to 500 mg or more, which may be administered in a manner consistent with the delivery of the drug and the disease state or condition to be treated. In the case of oral administration, active is generally administered from one to four times or more daily. Transdermal patches or other topical administration may administer drugs continuously, one or more times a day or less frequently than daily, depending upon the absorptivity of the active and delivery to the patient's skin. Of course, in certain instances where parenteral administration represents a favorable treatment option, intramuscular administration or slow IV drip may be used to administer active. The amount of active ingredient which is administered to a human patient is an effective amount and preferably ranges from about 0.05 mg/kg to about 20 mg/kg, about 0.1 mg/kg to about 7.5 mg/kg, about 0.25 mg/kg to about 6 mg/kg, about 1.25 to about 5.7 mg/kg.

The dose of a compound according to the present invention may be administered at the first signs of the onset of an autophagy mediated disease state, condition or symptom. For example, the dose may be administered for the purpose of lung or heart function and/or treating or reducing the likelihood of any one or more of the disease states or conditions which become manifest during an inflammation-associated metabolic disorder or tuberculosis or associated disease states or conditions, including pain, high blood pressure, renal failure, or lung failure. The dose of active ingredient may be administered at the first sign of relevant symptoms prior to diagnosis, but in anticipation of the disease or disorder or in anticipation of decreased bodily function or any one or more of the other symptoms or secondary disease states or conditions associated with an autophagy mediated disorder to condition.

### Examples

### Overview

Mammalian homologs of the yeast Atg8 protein (mAtg8s) are important in autophagy, but their exact mode of action remains to be defined. Recently, syntaxin 17 (Stx17), a SNARE with major roles in autophagy, was shown to bind mAtg8s. Here we broadened the analysis of potential mAtg8-SNARE interactions and identified LC3-interacting regions (LIRs) in several SNAREs. Syntaxin 16 (Stx16), and its cognate SNARE partners all have LIR motifs and bind mAtg8s. A knockout in STX16 caused defects in lysosome biogenesis whereas a double STX16 and STX17 knockout completely blocked autophagic flux and decreased mitophagy, pexophagy, xenophagy, and ribophagy. Mechanistic analyses revealed that mAtg8s and Stx16 maintained several aspects of lysosomal compartments including their functionality as platforms for active mTOR. These findings reveal a broad direct interaction of mAtg8s with SNAREs with impact on membrane remodeling in eukaryotic cells and expand the roles of mAtg8s to lysosome biogenesis.

A report of direct interaction between mAtg8s and Stx17 (Kumar et al., 2018) has set an unanticipated precedent, and here we explored whether there is a broader range of interactions between mAtg8s and mammalian SNAREs. Using bioinformatics and biochemical approaches, we found additional candidate SNAREs with LIR motifs that bind mAtg8s. Among the candidates, we focused on a cognate set of SNAREs, Stx16, Vti1a and Stx6, previously implicated in retrograde transport between endosomes and the trans-Golgi network (TGN). Through mutational and functional analyses, we found that Stx16 acts synergistically with Stx17 and is important for diverse types of autophagy, including mitophagy, pexophagy, ribophagy, and elimination of intracellular Mycobacterium tuberculosis. We have further found that Stx16 is important for cellular lysosomal content and function, and that mAtg8s modulate Stx16 localization on endolysosomal organelles. This uncovers a mechanism different from the previous views of how mAtg8s help grow autophagosomal and complete autolysosomal membranes. The inventors conclude that mAtg8s control autophagy at least in part by directly binding to SNARE proteins engaged in the biogenesis of the endolysosomal and autolysosomal organelles.

### Materials and Methods

### Antibodies and Reagents

Antibodies: rabbit anti-LC3B (L7543, for WB), rabbit anti-STX17 (HPA001204), and mouse anti-FLAG (F1804, for WB and IP) were from Sigma-Aldrich; mouse anti-LC3 (M152-3, for WB and IP) and rabbit anti-LC3 (PM036, for IF) were from MBL International; mouse anti-p62 (#610833) and mouse anti-GM130 (#610823) were from BD Biosciences; rabbit anti-STX16 (NBP1-92467), rabbit anti-VAMP3 (NB300-510), rabbit anti-VAMP4 (NBP2-13512), rabbit anti-VPS33A (NBP2-20872) and rabbit anti-TGN46 (NBP1-49643) were from Novus Biologicals; mouse anti-STX6 (H00010228-M01, for WB and IP) was from Abnova; mouse anti-Vti1a (sc-136117), mouse anti-VAMP8 (sc-166820) mouse anti-ATG3 (sc-393660) and mouse anti-β-actin (sc-47778) were from Santa Cruz Biotechnology, rabbit anti-VPS41 (ab181078), rabbit anti-PEX14 (ab183885), rabbit anti-PMP70 (ab85550), rabbit anti-GFP (ab290) and rabbit anti-β-tubulin (ab18251) were from Abeam; rabbit anti-LAMP1 (D2D11) (#9091), Phospho-AMPKα (Thr172) (#2531), AMPKα (#2532), Phospho-ULK1 (Ser757) (#6888), ULK1 (D8H5) (#8054), Phospho-4EBP1 (Thr37/46) (#2855), 4E-BP1 (#9452), Phospho-mTOR (Ser2448) (#2971), rabbit anti-mTOR (7C10) (#2981), rabbit anti-LKB1 (27D10) (#3050), and Autophagy Atg8 Family Antibody Sampler Kit (#64459) were from Cell Signaling Technology, mouse anti-LBPA (C64) (MABT837) was from EMD Millipore; mouse anti-DNA antibody (#61014) was from Progen; mouse anti-LAMP2 (H4B4) and mouse anti-M6PR (22d4) were from Developmental Studies Hybridoma Bank (DSHB) at the University of Iowa.

Earle's Balanced Salt Solution (EBSS) (E3024) and hydrogen peroxide solution (H1009) were from Sigma-Aldrich. Bafilomycin A1 was from InvivoGen (Cat Code: tlr1-baf1), CCCP (C2759), oligomycin A (#75351) and antimycin A (A8674) were from Sigma-Aldrich. LysoTracker™ Red DND-99 (L7528) and LysoTracker™ Green DND-26 (L7526) were from Thermo Fisher Scientific and used at 100 nM final concentration for 30 min.

### Cell Culture and Transfection

HeLa, HEK293T, U2OS and THP-1 cells wore from the American Type Culture Collection (ATCC). HeLa, HEK293T and U2OS cells were grown in Dulbecco's Modified Eagle's Medium (DMEM) supplemented with 10% fetal bovine serum (FBS), 2 mM L-glutamine, 10 mM HEPES, 1.0 mM sodium pyruvate, and 1×penicillin-streptomycin (Thermo Fisher Scientific) at 37° C. in a 5% CO₂ atmosphere. THP-1 cells were grown in RPMI 1640 with 2 mM L-glutamine adjusted to contain 1.5 g/L sodium bicarbonate, 4.5 g/L glucose, 10 mM HEPES and 1.0 mM sodium pyruvate and supplemented with 0.05 mM β-mercaptoethanol (M6250, Sigma-Aldrich) and 10% FBS. The Huh7 cell line was from Rocky Mountain Laboratory. Keima reporter cell lines HEK293 RPS3-Keima and HCT116 RPL28-Keima were from J. Wade Harper, Harvard University (An and Harper, 2018).

For Co-IP analysis of the interactions between over-expressed Stx16/Vti1a/Stx6 and mAtg8s, HEK293T cells in 10 cm petri dishes were transfected with corresponding plasmids via the ProFection Mammalian Transfection System (E1200, Promega). For other experiments, cells were transfected with Lipofectamine 2000 Transfection Reagent (#11668-019, Thermo Fisher Scientific) according to the manufacturer's manual.

### Plasmid Constructs

STX16 plasmid was from DNASU (# HsCD00396980); STX6 was from Addgene (#31581); VT11A DNA was PCR amplified from cDNA reverse transcribed from total RNAs isolated from HeLa cells. All constructs were transferred into pDEST-EGFP or pDEST-3×FLAG vectors using Gateway cloning system (#12535-019, Thermo Fisher Scientific), and verified by sequencing at GENEWIZ. EGFP-tagged LC3B and GABARAP constructed in pDEST vector were described before (Alemu et al., 2012). LIR-mutants of STX16 were generated with QuikChange Lightning Site-Directed Mutagenesis Kit (#210519, Agilent).

### GST Pull-Down Assay and Peptide Array Analysis

Recombinant GST and GST-fusion proteins were expressed in competent Escherichia coli SoluBL21 (Genlantis, # C700200) by inducing overnight bacterial cultures with 50-75 μg/mL isopropyl β-D-1-thiogalactopyranoside (IPTG). Expressed proteins were purified by immobilization on Glutathione Sepharose 4 Fast Flow beads (GE Healthcare, #17-5132-01). For GST pull-down assays, myc-tagged proteins were m vitro translated in the presence of radioactive methionine (³⁵S-methionine) using the TNT T7 Reticulocyte Lysate System (Promega, #14610). 10 μL of in vitro translated proteins were first precleared to remove unspecific binding with 10 μL of empty Glutathione Sepharose beads in 100 μL of NETN buffer (50 mM Tris pH 8.0, 150 mM NaCl, 1 mM EDTA, 0.5% NP-40) supplemented with cOmplete™ EDTA-free Protease Inhibitor Cocktail (Roche, #1183617001) for 30 min at 4° C. This was followed by incubation of the precleared mixture with purified GST or GST-fusion proteins for 1-2 h at 4° C. The mixture was washed five times with NETN buffer by centrifugation at 2500×g for 2 minutes followed by addition of 2×SDS gel-loading buffer (100 mM Tris pH 7.4, 4% SDS, 20% Glycerol, 0.2% Bromophenol blue and 200 mM dithiothreitol (DTT) (Sigma, # D0632) and heating for 10 minutes. The reaction was then resolved by SDS-PAGE and the gel stained with Coomassie Brilliant Blue R-250 Dye (Thermo Fisher Scientific, #20278) to visualize the GST and GST-fusion proteins. The gel was then vacuum-dried and radioactive signal detected by Bioimaging analyzer BAS-5000 (Fujifilm).

Peptide arrays of 20-mer amino acids of each SNARE protein were synthesized and immobilized on a membrane with the MultiPrep peptide synthesizer (Intavis Bioanalytical Instruments AG). The membrane was first blocked with 5% non-fat dry milk and then incubated with GST-GABARAP for 24 h. The membrane was washed three times and visualized by immunoblotting with anti-GST antibody as described (Rasmussen et al., 2019).

Generation of Knockout Cells with CRISPR/Cas9 gRNA

Sequences of gRNAs targeting STX16 and STX17 are shown in Fig. EV2A. ATG3 gRNA target sequence: TAGTCCACCACTGTCCAACA; STX6 gRNA target sequence: ACATGTCCCAGCGCATCGGA; VT11A gRNA target sequence: AGATACCACCCCAAAGTCGA. All the single KO and STX16/STX17 DKO HeLa, Huh7, U2GS, THP1 and HeLa-YFP-Parkin cells were generated by infecting target cells with single guide RNA (sgRNA) lentiviral vectors, lentiCRISPRv2, as described in (Sanjana et al., 2014). Briefly, HEK293T cells were transfected with lentiCRISPRv2 sgRNA vectors together with psPAX2 and pMD2.G at the ratio of 4 μg, 2.5 μg, 1.5 μg/6-cm dish. 60 hours later, the supernatants containing lentiviruses were collected and spun down at 1250 rpm for 5 min to clear cell debris. Lentiviruses were diluted with DMEM frill medium at 1:2 ratio and used to infect target cells overnight with the presence of 8 μg/mL of polybrene (Hexadimethrine bromide, H9268, Sigma-Aldrich) in 12-well plates. Then the medium with lentivirus was removed and changed to fresh medium for another 24 hours. Cells were selected with puromycin (1 μg/mL) for 5 days before validation of the knockout. Single clones of HeLa, Huh7 and U2GS KO cells were isolated by seeding single cells in 96-well plates after serial dilutions. For THP1 KO cells, it is impossible to isolate single clones due to their suspension nature, and thus depletion of Stx16 and Stx17 was not complete. Data is shown in Fig EV3C. Generation of LC3 TKO, GABARAP TKO and Hexa KO HeLa cells was described as before (Nguyen et al., 2016b).

### Western Blotting and Co-Immunoprecipitation (Co-IP)

Cell lysates were prepared with a standard procedure using RIPA buffer (Pierce, #89900) supplemented with cOmplete™ EDTA-free Protease Inhibitor Cocktail (Roche, #1183617001). Protein concentrations were determined using Pierce™ BCA Protein Assay Kit (#23225). Protein electrophoresis was carried out using TGX™ SDS Gels (Bio-Rad, #4561091), followed by blotting to a nitrocellulose membrane (Bio-Rad, #1620112). Membranes were incubated with primary antibodies diluted in blocking buffer (3% BSA diluted in 1×TBS buffer plus 0.05% Tween-20) overnight at 4° C. After incubation with fluorescently labeled secondary antibodies goat-anti-mouse IRDye 680LT or goat-anti-rabbit IRDye 800CW (LI-COR Biosciences) diluted 1:10 000 in blocking buffer, membranes were scanned using a fluorescence scanner Odyssey (LI-COR Biosciences).

For Co-IP of overexpressed proteins, HEK293T cells cultured in 10-cm dishes were lysed with 1 mL NP-40 cell lysis buffer (Invitrogen, FNN0021). Cell lysates were spun down at top speed for 10 min at 4° C. to clear cell debris. The supernatants were collected and incubated with 2.5 μL anti-FLAG antibody (M2) for 4 hours, followed by incubation with Dynabeads® Protein G for 1 hour, and precipitated proteins were subjected to Western blot analysis with rabbit anti-GFP antibody. For endogenous Co-IP analysis, cell lysates were prepared from cells cultured in 15-cm dishes, and incubated with 2.5 μg mouse anti-Stx6 or mouse anti-LC3 antibody overnight at 4° C., followed by Dynabeads® Protein G (Thermo Fisher Scientific, #10004D) binding and Western blot analysis of bound proteins.

### Immunostaining and Staining of Acidified Compartments for HCM and ImageStream Flow Cytometry

For HCM, cells were seeded in 96-well plates. After treatments, cells were washed once with 1×PBS, followed by fixation with 4% paraformaldehyde (PFA) for 5 min at room temperature. Cells were then permeabilized with 0.2% Triton X-100 for 10 min at room temperature. After incubation with blocking buffer (3% goat serum in 1×PBS), cells were stained with primary antibodies in blocking buffer for 1 h at room temperature or overnight at 4° C., followed by staining with secondary antibodies (Alexa Fluro conjugates. Thermo Fisher Scientific) diluted 1:1000 in blocking buffer for 1 h at room temperature. For LysoTracker Red DND-99, cells in 96-well plates were cultured in full medium or starved for 1 h in EBSS, followed by adding LTR for another 30 min at the final concentration of 100 nM. For ImageStream flow cytometry analysis of LysoTracker Green DND-26, cells in 6-cm dishes were starved in EBSS for 1 h, followed by adding LTG for another 30 min at the final concentration of 100 nM. Cells were collected by trypsinization and resuspended in 200 μL 1×PBS for Amnis analysis.

### High-Content Microscopy (HCM) Computer-Based Image Acquisition and Quantification

High-content microscopy with automated image acquisition and quantification was carried out using a Cellomics ArrayScan VTI platform (Thermo Fisher Scientific). A minimum of 500 primary objects (valid cells) per well were imaged for the quantifications of intracellular targets (regions of interest, ROI). Scanning parameters and object masks were preset with HCS Studio Scan software for algorithm-defined automated image acquisition and analysis. The images were viewed, and ROI quantified with HCS View software iDEV provided with the Cellomics (Mandell et al, 2014).

### ImageStream Flow Cytometry Analysis

ImageStreamX Mark II flow cytometer Amnis® (EMD Millipore) was used to analyze LysoTracker Green DND-26 (LTG) staining after autophagy induction by starvation. The 488 nm laser was used for excitation. Debris and doublets were gated out. Bright field (430-480 nm, Channel 01) and LTG (505-560 nm, Channel 02) channels were measured and at least 8,000 events of single cells per sample were collected. For analysis, the IDEAS version 6.0 software was used. Gating was applied to focused single cells using gradient root mean square of the bright field image then bright field area and aspect ratio, respectively, 50 cells with low/no fluorescent intensity and 50 cells with high fluorescent intensity and verified punctuate LTG staining by image collection from WT cells were selected to create a template. The template was then applied to all other samples in the same experiment to generate quantifications (counts of LTG/cell) for each sample.

### Electron Microscopy

Wild type or STX16/STX17^{DKO} HeLa cells were grown in 6-cm dishes until they became semi-confluent. The cells were starved for 2 hours in EBSS, followed by fixation with 2% glutaraldehyde (EM grade) in 0.2 M HEPES, pH 7.4. After 30 min initial incubation, cells were scraped under a small volume of fixative and transferred to Eppendorf tubes. The tubes were spun at full speed for 10 min at room temperature to get a firm pellet. The pellets were continued being fixed until total fixation time is 2 h. Thin sections were cut using an ultramicrotome, collected onto electron microscopy grids and stained with uranyl acetate and lead citrate. In order to count autophagic compartments or Golgi apparatus, 47 images for each sample were taken at primary magnification of 5000×, using the principles of uniform random sampling. The images were zoomed on computer screen. Autophagic compartments and the Golgi apparatus were counted, and the cytoplasmic area was estimated by point counting (Eskelinen, 2008).

M. tuberculosis Killing Assay

M. tuberculosis killing assay was carried out according to previously described (Kumar et al., 2018). Briefly, Mycobacterium tuberculosis Erdman (Erdman) culture was prepared by thawing frozen stock aliquot and grown in 7H9 Middlebrook liquid medium supplemented with oleic acid, albumin, dextrose and catalase (OADC, Becton Dickinson, Inc., Sparks, M D, USA), 0.5% glycerol and 0.05% Tween 80. Cultures were grown at 37° C. WT, STX16^{KO}, STX17^{KO}, and STX16/STX17^{DKO} THP1 cells (Fig. EV3) were infected with Erdman at MOI 10 and incubated with full medium for 18 h or 16 h followed by starvation in EBSS for 2 h to induce autophagy, lysed, and plated on 7H11 agar plates. CFU was enumerated 3 weeks later.

### Quantifications and Statistical Analysis

Data, means±SEM (n≥3), were analyzed with paired and unpaired two-tailed Student's/-test, or by one-way or two-way ANOVA (analysis of variance) followed by post hoc Tukey's test. Statistical significance was defined as: †, p≥0.05; *, p<0.05: **, p≤0.01.

### Results

### A Subset of SNAREs Including Syntaxin 16 Contain LC3-Interacting Regions

Previous studies have indicated that at least one SNARE involved in autophagy, Stx17, harbors a LIR motif that affects its distribution (Kumar et al., 2018). Here we carried out a broader analysis of all SNAREs for presence of putative LIR motifs using bioinformatics and conventional LIR motif consensus (Birgisdottir et al., 2013) followed up by biochemical assays (FIG. 1A and Appendix Table S1). We tested a subset of SNAREs containing putative LIRs in a screen with peptide arrays for binding to GST-GABARAP as a representative mAtg8 (FIG. 1A). Several SNAREs showed positive signals with GST-GABARAP in peptide arrays. These included only one R-SNARE, VAMP7, and mostly Qa-, Qb-, and Qc-SNAREs acting in different cellular compartments (Jahn & Scheller, 2006): Stx17, included as a positive control (Kumar et al., 2018) and already implicated in autophagy (Itakura et al., 2012), Stx18 acting in the ER (Hatsuzawa et al., 2000), GOSR1 functioning in the Golgi (Mallard et al., 2002, Subramaniam et al., 1996), Stx3 and Stx4 on plasma membrane (Low et al., 1996), Stx19 with no transmembrane domain and partitioning between cytosol and membranes (Wang et al., 2006), and Stx16, Stx6 and Vti1a, all acting in trafficking between endosomal compartments and TGN (Jahn & Scheller, 2006, Malsam & Söllner, 2011) (FIG. 1B and Fig. EV1A). Thus, a number of SNAREs may be binding partners for mAtg8s indicating a previously unappreciated potential for broader intersections between mAtg8 and SNARE systems.

Syntaxin 16 Directly Binds mAtg8s Through its LIR Motif

From the panel of SNAREs showing positive signals with GST-GABARAP in peptide arrays, we focused on a set of three cognate SNAREs, Stx16, Vti1a, and Stx6, known to form a complex (Malsam & Söllner, 2011). Co-immunoprecipitation (Co-IP) analyses of over-expressed fusion proteins confirmed that Stx16, Vti1a and Stx6 interact with LC3B and GABARAP (FIG. 1C). Co-IP analyses of endogenous proteins in HeLa and U2OS cells confirmed interactions between LC3 and Stx16 and Vti1a (Fig. EV1B). In GST pull-down assays with all 6 mAtg8 proteins, Stx16 showed a positive binding signal with LC3C, GABARAP, and GABARAPL1 (FIG. 1D). The peptide array binding results pointed to the ²¹⁹LVLV²²² residues in Stx16 (FIG. 1B), which resembled both the LC3C-preferring LIR of NDP52 (ILVV) (von Muhlinen et al., 2012) and the GABARAP-interaction motifs with important Val residues in second and fourth positions of the core LIR motif (Rogov et al., 2017). This motif was found in the linker region between the Habc and SNARE domains of Stx16 matching the requirement to be outside of known structured regions (Popelka & Klionsky, 2015). When we mutated L-219 and V-222 in the predicted (albeit atypical) LIR motif (²¹⁹LVLV²²² mutated to ²¹⁹AVLA²²²) of Stx16, this reduced Stx16 binding to LC3C and GABARAP (FIG. 1E). Additionally, we changed both Val residues within the ²¹⁹LVLV²²² motif to Ala, and the resulting Stx16 variant showed reduction in binding to LC3C and GABARAP comparable to the ²¹⁹AVLA²²² mutant in GST pull-down experiments (FIG. 1F). Finally, when we mutated all 4 residues within the ²¹⁹LVLV²²² motif, the association between Stx16 and LC3C or GABARAP was completely abolished (FIG. 1F).

Complementary to the above experiments with LIR mutations, we tested whether the previously defined LIR-docking site (LDS) in GABARAP (Behrends et al., 2010, Birgisdottir et al., 2013) was responsible for binding to Stx16. Two conserved hydrophobic pockets (HP1 and HP2) are known to define the LIR-docking site of GABARAP (Behrends et al., 2010). The Y49A mutation in the HP1 pocket of GABARAP reduced its interaction with Stx16, whereas the complete LDS mutant (double HP1 and HP2 Y49A/F104A mutation) abrogated GABARAP's binding to Stx16 in GST pull-down assays (FIG. 1G). Thus, GABARAP and Stx16 interaction depends on the canonical LDS motif in GABARAP.

### Syntaxin 16 is Required for Efficient Autophagy Flux in Response to Starvation

Stx17 has been implicated in mammalian autophagosome-lysosome fusion (Itakura et al., 2012). However, additional SNAREs have now been shown to contribute to this process (Matsui et al., 2018), but relative contributions remain to be fully defined. We generated CRISPR/Cas9-mediated STX17 knockout (KO) in different cell lines (HeLa and Huh7; Fig. EV2A and B) and analyzed autophagy flux by monitoring LC3-II levels (FIG. 2A and B; Fig. EV2C and D). In STX17^{KO} HeLa cells, the autophagic flux continued at almost the same rates as in wild type (WT) control cells, as shown by slight increase of LC3-II levels upon starvation (FIG. 2A). A similar effect was observed in hepatocellular carcinoma Huh7 STX17^{KO} cells, which in general displayed faster LC3-II flux (Fig. EV2C and D). Knocking out Stx17 did not cause any significant change in the levels of p62, an autophagic receptor that is degraded upon starvation (Bjorkoy et al., 2005) and is often used as readout of autophagic flux (Klionsky et al., 2016) (FIG. 2A and Fig. EV2C).

When we knocked out STX16 in HeLa cells, this too had no major effects on LC3-II flux (Fig. EV2A and B; FIGS. 2C and D). However, when a double STX16/STX17knockout was generated (Fig. EV2B), the LC3 flux was completely abrogated, with LC3-II levels in starvation-induced cells accumulating at similar levels as in cells treated with bafilomycin A1 (FIGS. 2C and D), usually considered as a complete blocker of autophagic flux (Klionsky et al., 2016). The LC3-II accumulation could be partially relieved by complementation of STX16/STX17^{DKO} cells transfected with FLAG-tagged WT Stx16 (Fig. EV2E). Given that this indicated a block in autolysosome production, we examined STX16/STX17^{DKO} cells using automated quantitative high-content microscopy (HCM) and tandem mCherry-EYFP-GABARAP, by monitoring and quantifying overlap between red (mCherry) and green (EYFP) fluorescence as another measure of autophagic flux. The overlap between mCherry and EYFP was significantly increased in STX16/STX17^{DKO} cells compared to that in WT cells upon starvation in EBSS (Fig. EV2F and G). At the ultrastructural level, electron microscopy quantification of initial (A Vi) vs. degradative (AVd) autophagic vacuoles (Eskelinen, 2008, Tanaka et al., 2000) also indicated accumulation of AVi vesicles (FIGS. 2E and F). In conclusion, absence of either Stx16 or Stx17 alone cannot block completion of the autophagy pathway, but when both are ablated, this prevents progression to autolysosomes.

### Syntaxin 16 and Syntaxin 17 are Required for Completion of Diverse Selective Autophagy Processes

Mitophagy is a very well-studied process of selective autophagy and is often used as an example to study elimination of defunct organelles (Youle & Narendra, 2011). To further characterize effects of STX16/STX17 double knockout, we employed HCM quantification of mitochondrial DNA (mtDNA) as a measure of cellular mitochondrial content often used in mitophagy studies (Lazarou et al., 2015, Nguyen et al., 2016b). We generated STX16/STX17 DKO in HeLa cells stably expressing YFP-Parkin (Lazarou et al., 2015) (Fig. EV3A). We then stressed mitochondria using two different depolarizing agents: CCCP or oligomycin A+antimycin A (OA), and quantified Parkin-dependent mitophagy in STX16/STX17^{DKO} HeLa/YFP-Parkin cells. In wild type cells, CCCP or OA caused a major reduction in mtDNA (FIGS. 3A and B). Loss of Stx16 and Stx17 caused significant reduction in mitophagy (FIGS. 3A and B). Clearly, additional processes were also involved as STX16/STX17^{DKO} HeLa/YFP-Parkin cells still eliminated mitochondria probably through additional mechanisms (Nguyen et al., 2016a). Previous studies indicated that Stx17 is not important for mitophagy (Nguyen et al., 2016b). Nevertheless, a loss of Stx16 in the STX17^{DKO} background affected mitophagy significantly.

Pexophagy is another classical example of selective autophagy (Gatica et al., 2018, Manjithaya et al., 2010). We monitored pexophagy in cells treated with H₂O₂ (Zhang et al., 2015) using liver cell line Huh7, which expresses wild type LKB1 that can activate AMPK (Tan et al., 2018) (Fig. EV3B). We monitored pexophagy by quantifying levels of PEX14 and PMP70 (Zhang et al., 2015) proteins, and detected decrease in these peroxisomal constituents over a period of 6 hours of exposure to H₂O₂ (FIG. 3C). In cells lacking both Stx16 and Stx17, this reduction in peroxisomal protein levels was abrogate (FIGS. 3C and D). Thus, STX16/STX17 DKO significantly inhibited pexophagy.

Autophagic defense against intracellular microbes, such as Mycobacterium tuberculosis (Mtb) (Gutierrez et al., 2004), sometimes referred to as xenophagy, is considered to be another form of selective autophagy. We tested the survival of M. tuberculosis in macrophage-like cell line THP1 and compared the effects of Mtb killing in WT, single STX16^{KO} or STX17^{KO} and STX16/STX17^{DKO} cells (Fig. EV3C). As previously reported (Gutierrez et al., 2004), induction of autophagy by starvation reduced survival of Mtb in WT THP1 cells (FIG. 3E). However, this effect was lost in STX16^{KO} THP1 cells (FIG. 3E; Fig. EV3C). Further analyses of combination CRISPR KO mutants of STX17 alone, previously reported in THP1 cells to play a role in control of intracellular Mtb (Kumar et al., 2018), or both STX16 and STX17, showed no further additive effects between Stx16 and Stx17 (FIG. 3E).

Taken together, these data indicate that STX16/STX17 DKO significantly reduces mitochondrial (Lazarou et al., 2015), peroxisomal (Manjithaya et al., 2010), and bacterial (Mtb) (Gutierrez et al., 2004) clearance under conditions known to induce autophagy (FIG. 3F).

### Keima-Based Ribophagy Probes Indicate a Role of Stx16 in Autophagic Maturation

Keima fluorescent protein has become an important tool for detection of the arrival of autophagy substrates to autolysosomes (Katayama et al., 2011). In functional autolysosomes, despite being in a degradative compartment, Keima protein is stable but changes its spectral properties (illustrated in FIG. 4A) from maximum excitation wavelength at 440 nm in neutral pH to 586 nm in acidic pH, with emission remaining constant at 620 nm (Violot et al., 2009). These shifts can be monitored using 440 nm and 560 nm excitation filters and emission at 620 nm in an HCM Cellomics system (see Methods). We applied Keima to monitor ribophagy (An & Harper, 2018), and generated STX16/STX17 DKO using CRISPR/Cas9 (FIG. 4B) in HEK293 cells stably expressing RPS3-Keima (fusion between 40S ribosomal protein S3 and mKeima) protein (An & Harper, 2018). RPS3-Keima was diffuse cytoplasmic in untreated cells. Under starvation conditions, we examined ribophagy as previously reported (An & Harper, 2018), by monitoring progression of RPS3-Keima into acidic autolysosomal compartments via quantification of cytoplasmic puncta at 560 nm excitation and 620 emission wavelengths (FIGS. 4C and D). The HEK293 STX16/STX17^{DKO} cells showed significantly fewer autolysosomal puncta originating from RPS3-Keima than in parental WT cells (FIGS. 4C and D). An independent set of experiments was carried out with HCT116 cells stably expressing a fusion between 60S ribosomal protein L28 and mKeima (RPL28-Keima). The inventors generated STX16/STX17 DKO in these cells as well using CRISPR/Cas9 (FIG. 4B). Progression of ribophagy in HCT116 RPL28-Keima STX16/STX17^{DKO} cells was diminished relative to parental WT cells (FIGS. 4E and F). Thus, tire use of previously characterized Keima ribophagy probes (An & Harper, 2018) further confirmed the importance of having both Stx16 and Stx17 for autophagic cargos to arrive into acidified compartments, including during ribophagy.

### Syntaxin 16 Plays a Role in Lysosomal Biogenesis

When the inventors tested autophagy response by monitoring endogenous LC3 puncta, we found an increase in LC3 dots in STX16 knockout cells (HeLa STX16^{KO}) relative to WT cells, under basal conditions and in EBSS-starved cells (FIGS. 5A and B). Next, we determined by HCM the overlap area between LC3 and LAMP2 profiles, and found that the overlap was strongly reduced in HeLa STX16^{KO} vs WT cells (FIGS. 5A and C). When we quantified LAMP2 puncta/cell, we found that LAMP2⁺ puncta were diminished in HeLa STX16^{KO} cells (FIGS. 5A and D). Thus, the appearance of reduced colocalization (overlap area by HCM) between LC3 and LAMP2 was not a result of fewer autolysosomes but was primarily due to the overall reduction of LAMP2⁺ organelles in STX16^{KO} cells (FIGS. 5A and D), a phenotype that could be complemented by reintroducing WT Stx16 but less so with a LIR mutant of Stx16 (Appendix Fig S1A and B). The reduction in cytoplasmic content of LAMP2⁺ organelles in HeLa STX16^{KO} cells was also seen by immunoblots, showing reduced total LAMP1 and LAMP2 protein levels in cells knocked out for Stx16 (FIG. 5E). This was further tested by generating STX16 knockout in different cell lines (Fig. EV2A and B). As with different clones of HeLa STX16^{KO} cells, different single clones of Huh7 STX16^{KO} and osteosarcoma U2OS STX16^{KO} cells had reduced LAMP1 and LAMP2 protein levels (FIG. 5E). Thus, Stx16 is important for the maintenance of homeostatic LAMP levels. We also generated CRISPR knockouts for Stx6 and Vti1a, and found reduced levels of LAMP2 in STX6^{KO} and VT11A^{KO} HeLa cells (FIG. 5F). Thus, Stx16 and its cognate Qb- and Qc-SNAREs affect cellular LAMP levels.

Prior ultrastructure studies have implicated HOPS component VPS41 in LAMP1 and LAMP2 transport from the TGN to late endosomal compartments (Pols et al., 2013). In STX16^{KO} HeLa cells, there was increased colocalization of detectable LAMP2 with the TGN marker TGN46, normalized for LAMP puncta in HCM quantifications (Fig. EV3D and E). We thus tested whether Stx16 and its cognate Qb- and Qc-SNAREs, Vti1a and Stx6 (both showing positivity in mAtg8 peptide binding arrays: FIG. 1A) interact with VPS41. Using FLAG-tagged Stx6 and Stx16 we detected VPS41 in FLAG immunoprecipitates (Fig. EV3F). We found VPS41 but not VPS33A (a component of the HOPS complex) in endogenous protein complexes with Stx6, and Vti1a (Appendix Fig. S1C). These observations suggest a connection of Stx16 (plus Vti1a and Stx6) to VPS41 and the transport of lysosomal proteins such as LAMP1 and LAMP2, previously proposed specifically for VPS41 and not other components of HOPS (Pols et al., 2013).

Starvation Increases Interactions of Stx16 with the Lysosomal R-SNARE VAMP8

The inventors found that Stx16 interacts with VAMP3 and VAMP4 (Fig. EV3F), consistent with the known literature regarding VAMP3 and VAMP4 as cognate R-SNAREs during retrograde transport from endosomes to TGN (Ganley et al., 2008, Mallard et al., 2002). However, upon starvation another R-SNARE, VAMP8, increased its presence in Stx16 complexes (Fig. EV3F). The lysosomal SNARE VAMP8 (Jahn & Scheller, 2006) has not been previously implicated in Stx16-dependent trafficking, and our findings suggest a new relationship detected only under starvation conditions. Similarly, Stx6, a Qc-SNARE working together with Stx16, showed increased interactions with VAMP8 in starved cells (Fig. EV3F). Thus, similar relationship to VAMP8 was observed with both Stx16 and Stx6. We also noticed increased Stx6-VAMP3 interactions in starved cells (Fig. EV3F), and interpret this as an overall increase in trafficking between TGN and the lysosomal/endosomal pathway.

Stx16 Affects mTOR Regulation

Lysosomes are not only digestive organelles but also serve as a principal cytoplasmic location for active mTOR (Betz & Hall, 2013, Lawrence & Zoncu, 2019, Saxton & Sabatini, 2017). Given that the inventors detected defects in LAMP1/2 levels and profiles (FIGS. 5D and E) in STX16^{KO} cells, the inventors wondered whether mTOR would be affected and serve as another reporter of the lysosomal status in this context. The inventors tested mTOR activity by monitoring phosphorylation of its downstream targets, such as 4E-BP1 (Gingras et al., 1999), and ULK1^{S757}, with latter being a principal phospho-site for ULK1 and repression of autophagy by mTOR (Kim et al., 2011). Under resting conditions, no differences in mTOR activation status were observed in STX16^{KO} WT cells (FIG. 5G). Under starvation conditions, as expected, mTOR activity was reduced, but this reduction was much more pronounced in STX16^{KO} cells (FIG. 5G).

In resting cells, active mTOR forms cytoplasmic puncta and localizes to lysosomes (Betz & Hall, 2013, Saxton & Sabatini, 2017), but in starved cells mTOR is less punctate and translocates from lysosomes into the cytoplasm (Sancak et al., 2010). Under resting conditions (full medium), mTOR puncta (quantified by HCM) were not reduced in STX16^{KO} vs WT cells (FIGS. 5H and I). One hour of starvation reduced mTOR puncta, as quantified by HCM (FIGS. 5H and I), reflecting mTOR inactivation. This effect was more pronounced in STX16^{KO} cells relative to WT cells (FIGS. 5H and I). When the cells were starved for 6 hours, a time point that coincided with mTOR persistent inactivation assessed by phosphorylation of its targets (FIG. 5G), we found an even stronger reduction in total mTOR puncta (Appendix Fig. S2A and B). Localization of mTOR to LAMP2⁺ profiles was reduced by starvation (examined at 6 h; Fig. EV4A and B), an effect that was more pronounced in STX16^{KO} relative to WT cells. Thus, Stx16 affects mTOR activation state, correlating with the role of Stx16 in cellular lysosomal status.

### Stx16 is Required for Appropriate Distribution of Acidified Compartments in the Cell

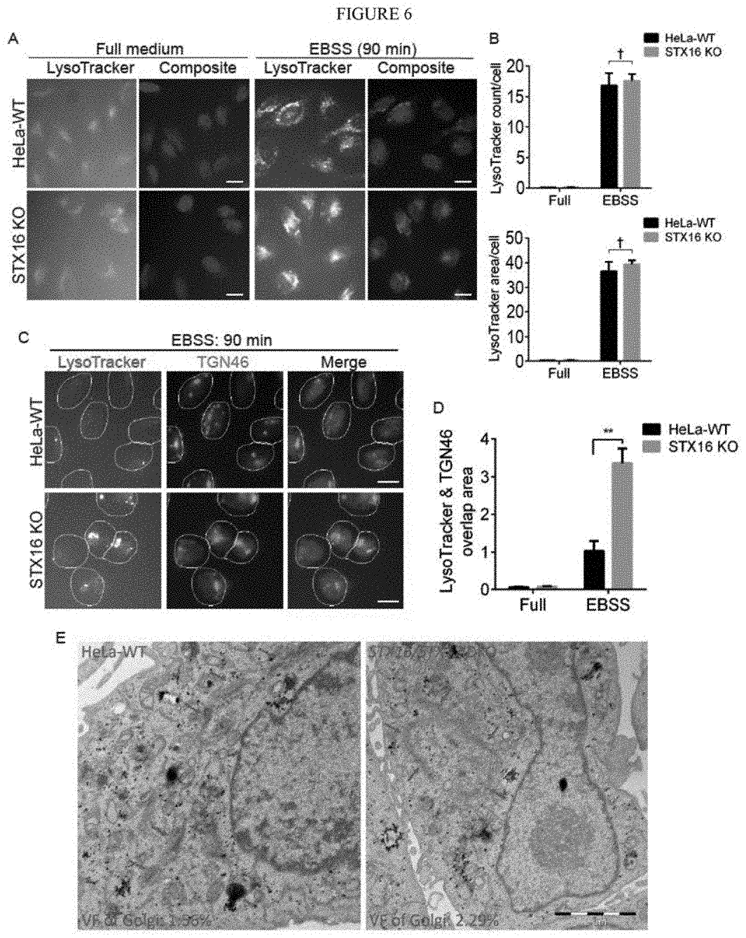

Since STX16 knockout resulted in reduced cellular content of LAMP1/2, which is in lysosomal and additional acidified endosomal compartments (Lippincott-Schwartz & Fambrough, 1987) (Cheng et al., 2018), we anticipated that the cells might have diminished content of acidified organelles. We used LysoTracker Red DND-99 (LTR) as a standard cell biological probe for acidification of intracellular compartments (Wubbolts et al., 1996). Surprisingly, we found no change in total LTR fluorescence by number of profiles/cell or total area/cell (FIGS. 6A and B) utilizing HCM (Cellomics) and comparing WT and STX16^{KO} HeLa cells under autophagy-inducing conditions (starvation). This was confirmed by total LysoTracker Green DND-26 (LTG) fluorescence and LTG puncta/cell using ImageStream flow cytometry (Amnis) in WT vs STX16^{KO} cells induced for autophagy (Appendix Fig. S3A-C). However, when we examined localization and morphology of LTR⁺ profiles in STX16^{KO} cells, under autophagy-inducing conditions when LTR⁺ organelles were strongly enhanced (relative to the resting conditions), we found that the LTR labeling redistributed from its usual presentation as cytoplasmic puncta into a compartment clustering in a perinuclear region colocalizing with the TGN marker TGN46 (FIGS. 6C and D). To ensure that the Golgi apparatus was not perturbed, we re-examined the ultrastructural images in FIG. 2E, and found that the overall morphology of the Golgi stacks was similar in WT and mutant cells, but that the volume fraction of the Golgi compartment was increased (FIG. 6E), consistent with an increased retention of acidified compartments in the Golgi. Furthermore, TGN46 did not show major changes in distribution relative to the Golgi marker GM130 (Fig. EV4C and D) and relative to another factor in TGN-endolysosomal trafficking mannose 6-phosphate receptor/M6PR (Fig EV4E and F). Thus, the effects of Stx16's absence entail redistribution of acidified compartments, in keeping with a reduction in levels of the integral lysosomal membrane proteins LAMP1 and LAMP2.

### Mammalian Atg8s Regulate Stx16 Localization to Endosomal and Lysosomal Compartments

Since Stx16 is an mAtg8-binding SNARE (FIG. 1), we tested whether mAtg8s influenced Stx16 localization in the cell. We used the previously characterized triple LC3 A 3,C knockout (Tri-LC3^{KO}), triple GABARAP, -L1, -L2 knockout (Tri-GBRP^{KO}), and all six mATG8s knockout (Hexa^{KO}) HeLa cells (Nguyen et al., 2016a) (Fig. EV5A). First, we tested LAMP1 and LAMP2 protein levels in Tri-LC3^{KO}, Tri-GBRP^{KO} and Hexa^{KO} cells. LAMP levels were not reduced under resting conditions in mAtg8 mutant cells by quantifying LAMP2⁺ organelles (HCM, number of puncta/cell and total area/cell) (Appendix Fig. S4A and B) as well as by Western blot analysis of LAMP1/2 proteins (Appendix Fig S4C). We nevertheless observed a minor LAMP reduction, possibly reflecting “exhaustion” during long-term (8 h) starvation, in Tri-GBRP^{KO} and Hexa^{KO} cells (Appendix Fig. S4D). When we assessed localization of Stx16 to LAMP2 (lysosome) and LBPA (late endosome), we observed significantly reduced colocalizations between Stx16 and LAMP2 as well as Stx16 and LBPA by several HCM measures: (i) LAMP2-Stx16 overlap (FIGS. 7A and B); and (ii) LBPA-Stx16 overlap (FIG. 7C; Appendix Fig. S5A). Comparing STX16 WT and its ²¹⁹LVL²²², ²¹⁹AVLA^{22Z}, or LIR-4A mutants, the LIR mutants showed reduced localization to LAMP2 profiles when compared to WT (Fig EV5B and C). Thus, mAtg8s and their binding site (LIR) on Stx16 play a role in placement of Stx16 on lysosomes.

Endogenous Co-IP analyses showed reduced interactions between components of the Stx16/Vti1a/Stx6 SNARE complex in Tri-GBRP^{KO} and Hexa^{KO} cells (Fig EV5D), suggesting a regulatory role mAtg8s in SNARE complex assembly or stability. A lipidation status of mAtg8s may be of significance as well, since we observed reduced interactions between Stx6 and Stx16, Vti1a or VPS41 in ATG3^{KO} HeLa cells generated by CRISPR/Cas9 (Fig. EV5E); ATG3 is a key factor required for lipidation of mAtg8s (Mizushima et al., 2011). We next tested cellular acidified compartments by HCM and Amnis, and quantified effects of mAtg8 mutants, hr Hexa⁸⁰ cells, the overall LTR⁺ profiles were reduced by HCM (FIGS. 7D and E) and LTG⁺ profiles were reduced by ImageStream flow cytometry (Appendix Fig. S5B-D). mAtg8s also had an effect on mTOR inactivation in response to starvation, as evidenced by a more pronounced loss of phosphorylation of mTOR targets ULK1 (phospho-Ser757) and 4E-BP1 (phospho-Thr37/46) in Hexa^{KO} cells relative to WT HeLa cells (Fig. EV5F). In summary, albeit mAtg8s do not completely parallel Stx16 phenotype, they do have an effect on Stx16 distribution and mTOR inactivation. Additionally, mAtg8s show strong effects on acidified, LTR⁺ compartments in cells under autophagy inducing conditions. Thus, mAtg8s and Stx16 have partially overlapping effects on the endolysosomal system.

### Discussion

Pursuant to the present invention, the inventors uncovered a broad potential for interactions between the core autophagy components, mAtg8s, and the core membrane fusion system composed of SNAREs. The bioinformatics analyses and follow-up biochemical experiments indicate that mAtg8s and several SNAREs work together in membrane trafficking and organelle biogenesis. One subset of SNAREs interacting with mAtg8s, Stx16-Vti1a-Stx6, was examined in detail, and Stx16 was fully characterized for mAtg8 binding, as has been done for Stx17 (Kumar et al., 2018). This leads us to propose a model departing from the conventional views of how LC3 or other mAtg8s work in autophagy, suggesting a new mechanism of control of membrane trafficking/fusion through direct action of mAtg8s on SNARE molecules (FIG. 7F).

The inventors' findings with Stx16 indicate a function for mAtg8s in the maintenance of lysosomal compartments (FIG. 7F), through binding directly and regulating SNAREs of the Stx16 complex, which leads up to autolysosome biogenesis. This amends the conventional view of mAtg8s expanding the autophagosomal membranes (Carlsson & Simonsen, 2015) and complements more recent views of mAtg8s working in the recruitment of various components of the autophagic apparatus. These include: (i) mAtg8 binding to autophagic receptors from the SLR (Birgisdottir et al., 2013, Rogov et al., 2014) and TRIM (Keown et al., 2018, Maudell et al., 2014) families of receptors; (ii) the ULK1-FIP200-ATG13 autophagy initiation complex (Aleinn et al., 2012); (iii) a large number of ancillary components facilitating the autophagosome-lysosome fusion (Kriegenburg et al., 2018, McEwan et al., 2015, Olsvik et al., 2015, Wang et al., 2016); and (iv) non-autophagic roles of mAtg8s, such LAP (Sanjuan et al., 2007), LAP-like processes (Florey et al., 2015), MVB/exosome formation (Guo et al., 2017), and via binding to TECPR2 in the COPII-dependent ER export (Stadel et al., 2015).

Human cells have 6 mAtg8s, with proposed but not hilly delineated specialized functions (Shpilka et al., 2011). Their role has been established in the recruitment of several important ancillary factors facilitating membrane fusion at the autophagosome-lysosome fusion stage of the autophagic pathway (Nakamura & Yoshimori, 2017). An early study has shown GABARAPL2/GATE-16 interaction with NSF enhancing its ATPase activity thus implicating it in activation of the Golgi SNARE GOS-28/GOSR1 (Sagiv et al., 2000), of consequence for post-mitotic Golgi reformation (Muller et al., 2002) and intra-Golgi transport (Sagiv et al., 2000). This is in keeping with the results of our screen of mAtg8-interacting SNAREs and is compatible with the concept of mAtg8s directly acting on SNAREs.

Stx16 and its cognate SNAREs have been shown to play a role in retrograde trafficking from endosomal compartments to TGN with two distinct pairings of Qa-, Qb-, and Qc-SNAREs: (i) Stx16, Vti1a, Stx10, required specifically for M6PR transport (we did not observe effects on M6PR relative to TGN46 in our studies), which impacts trafficking of lysosomal hydrolases (Ganiev et al., 2008); and (ii) Stx16, Vti1a, and Stx6, which is independent of the Stx10/M6PR pathway and controls trafficking to TGN of endocytosed incoming toxins bound to membrane glycoconjugates (Mallard et al., 2002). There is also a Stx16-independent retrograde pathway that instead depends on GOS-28/GOSR1 (Tai et al., 2004), another SNARE that incidentally binds mAtg8s (see FIG. 1A, 6^{th} peptide array from the top). Of note, GOS-28/GOSR1 has been first described as a GATE-16-binding SNARE (Sagiv et al., 2000) with Golgi functions. Most of the prior studies with Stx16 complexes have been focused on retrograde trafficking to the TGN, and thus our observations could be due to perturbed retrograde membrane and cargo flow. However, our findings that Stx16 is in complexes with VPS41, previously implicated in anterograde transport of LAMP1/2 (Pols et al., 2013), and reduced abundance of LAMP1/2⁺ organelles in STX16^{KO} cells suggest that Stx16 defect blocks the prograde delivery of LAMP1/2 during lysosomal biogenesis.

Trafficking of various components that make up functional lysosomes is only partially understood (Bright et al., 2016, Luzio et al, 2000, Saftig & Klumpennan, 2009) especially regarding anterograde pathways to lysosomes of integral membrane proteins such as LAMP1/2 and trafficking to and assembly at the lysosome of the V₁V₀ holoenzyme vacuolar H⁺-ATPase (Saftig & Klumperman, 2009). Our findings suggest that Stx16 is responsible for LAMP1/2 trafficking to lysosomes with secondary consequences on the overall levels of LAMPs when this delivery is disrupted. It is likely that Six 16 does not affect vacuolar H⁺-ATPase's ability to function but these acidified compartments may be displaced due to a lack of clearly defined lysosomal destination. Some of STX16^{KO} phenotypes (perturbation of acidified compartments) are echoed in mAtg8s null Hexa^{KO} cells, which show diminished overall content of acidified organelles. These phenomena can in part be explained by mAtg8s affecting Stx16 localization. Since Hexa^{KO} effects on acidification are more pronounced than the effects of STX16^{KO}, this indicates additional functions for mAtg8s, specifically in H⁺-ATPase function. For example, it has recently been shown that LC3 affects ATP6V1E1 subunit of the V₁ domain of vacuolar H⁺-ATPase (Guo et al., 2017). Although mAtg8s do not phenocopy in full the Stx16 phenotype, they do affect Stx16 distribution and mTOR activity. Thus, mAtg8s and Stx16 have only partially overlapping effects on the endolysosomal system most likely due to mAtg8 action exceeding the reach of the Stx16 function.

Since lysosomes are the location of active mTOR (Betz & Hall, 2013, Saxton & Sabatini, 2017), we used mTOR activity as another readout of Stx16 effects on the lysosomal function. Somewhat surprisingly, given the strong reduction of LAMP1/2 levels and diminished number of LAMP⁺ organelles, STX16^{KO} did not show effects on basal mTOR activity. However, during starvation, which inactivates mTOR and triggers its translocation from the lysosomes. mTOR was inhibited far more prominently in STX16^{KO} cells and this was also reflected in fewer mTOR⁺ cytoplasmic puncta. One interpretation is that the reduced LAMP1/2 content differentially affects subpopulations of lysosomes (Bright et al., 2016) and that the ones that are platforms for active mTOR are preferentially preserved/maintained. Nevertheless, upon stress and perturbance, inactivation of mTOR is more effective and more durable in STX16^{KO} cells. This could be a reflection of altered trigger thresholds dependent on diminished lysosomal sources of nutrients and/or difficulties in re-establishing nutrient equilibrium and mTOR activity. Alternatively, Stx16 may be important in transport to lysosomes of additional mTOR regulatory components important for its reactivation.

The Qa-SNARE Stx17 was first appreciated as a key SNARE in autophagosome-lysosome fusion (Diao et al., 2015, Guo et al., 2014, Itakura et al., 2012, Takats et al., 2013, Wang et al., 2016). Recent studies have uncovered the yeast SNARE, Ykt6, as important in autophagosome-vacuole/lysosome fusion in yeast (Bas et al., 2018, Gao et al., 2018), and that mammalian Ykt6 ortholog (Matsui et al., 2018, Takats et al., 2018) plays a potentially dominant (over Stx17) role in mammalian autophagosome-lysosome fusion. Whereas the exact mechanism of Ykt6 in maturation has not been agreed upon, with differences in proposed models (Matsui et al., 2018, Takats et al., 2018), it is worth noting that Ykt6 is also part of one of retrograde trafficking routes from endosomes to TGN that includes GOS-28/GOSR1 (Tai et al., 2004), an mAtg8-binding SNARE as discussed above. Nevertheless, our data here suggest that Stx17 still plays a very important role in autophagosomal flux, revealed in the requirement for a STX16/STX17 double KO to block autophagic flux using the conventional and well accepted LC3 flux assay (Klionsky et al., 2008), and reflected in autophagic degradation of a diverse panel of substrates: mitochondria, peroxisomes, M. tuberculosis, and ribosomes.

Stx17 interacts via its LIR motif with mAtg8s, and this may play a role in its recruitment to autophagosomes as a prelude to fusion with the lysosomes (Kumar et al., 2018). However, Stx17's role in autophagy begins much earlier, during initiation (Arasaki et al., 2018, Arasaki et al., 2015, Hamasaki et al., 2013, Kumar & al., 2019) ensuring drat once autophagosomes are formed they are enabled for fusion with endolysosomal compartments. The present screen for other SNAREs that bind mAtg8s yielded a number of candidates in addition to Stx16, such as Vti1a and Stx6, which partner with Stx16, and Stx3 and Stx4, SNAREs that are engaged in secretory autophagy (Kimura et al., 2017). This prompts us to suggest that mAtg8s' function in autophagy should be reconsidered and some of its mechanistic principles revisited. We propose that mAtg8s act by reshaping overall membrane trafficking in the cell through direct interactions and effects on specific SNAREs and redirect membrane flow toward autophagy at its initiation and termination stages.
Alemu E A, Lamark T, Torgersen K M, Birgisdottir A B, Larsen K B, Jain A, Olsvik H, Overvatn A, Kirkin V, Johansen T (2012) ATG8 family proteins act as scaffolds for assembly of the ULK complex: sequence requirements for LC3-interacting region (LIR) motifs. J Biol Chem 287: 39275-90
An H, Harper J W (2018) Systematic analysis of ribophagy in human cells reveals bystander flux during selective autophagy. Nature cell biology 20:135-143
Arasaki K, Nagashima H, Kurosawa Y, Kimura H, Nishida N, Dohmae N, Yamamoto A, Yanagi S, Wakana Y, Inoue H, Tagaya M (2018) MAP1B-LC1 prevents autophagosome formation by linking syntaxin 17 to microtubules. EMBO Rep
Arasaki K, Shimizu H, Mogari H, Nishida N, Hirota N, Furuno A, Kudo Y, Baba M, Baba N, Cheng J, Fujimoto T, Ishihara N, Ortiz-Sandoval C, Barlow L D, Raturi A, Dohmae N, Wakana Y, Inoue H, Tani K, Backs J B et al. (2015) A role for the ancient SNARE syntaxin 17 in regulating mitochondrial division. Dev Cell 32:304-17
Bakula D, Muller A J, Zuleger T, Takacs Z, Franz-Wachtel M, Thost A K, Brigger D, Tschan M P, Frickey T, Robenek H, Macek B, Proikas-Cezanne T (2017) WIPI3 and WIPI4 beta-propellers are scaffolds for LKB1-AMPK-TSC signalling circuits in the control of autophagy. Nat Commun 8: 15637
Bas L, Papinski D, Licheva M, Torggler R, Rohringer S, Schuschnig M, Kraft C (2018) Reconstitution reveals Ykt6 as the autophagosomal SNARE in autophagosome-vacuole fusion. J Cell Biol
Baskaran S, Carlson L A, Stjepanovic G, Young L N, Kim do J, Grub P, Stanley R E, Nogales E, Hurley J H (2014) Architecture and dynamics of the autophagic phosphatidylinositol 3-kinase complex. Elife 3
Behrends C, Sowa M E, Gygi S P, Harper J W (2010) Network organization of the human autophagy system. Nature 466:68-76
Betz C, Hall M N (2013) Where is mTOR and what is it doing there? J Cell Biol 203: 563-74
Bhujabal Z, Birgisdottir A B, Sjottem E, Brenne H B, Overvatn A Habisov S, Kirkin V, Lamark T, Johansen T (2017) FKBP8 recruits LC3A to mediate Parkin-independent mitophagy. EMBO Rep 18:947-961
Birgisdottir A B, Bhujabal Z, Wirth M, Sjottem E, Evjen G, Zhang W, Lee R, O'Reilly N, Tooze S A Lamark T, Johansen T (2019) Members of the autophagy class in phosphatidylinositol 3-kinase complex I interact with GABARAP and GABARAPL1 via LIR motifs. Autophagy In press
Birgisdottir A B, Lamark T, Johansen T (2013) The LIR motif—crucial for selective autophagy. Journal of cell science 126: 3237-47
Bjorkoy G, Lamark T, Brech A, Outzen H, Perander M, Overvatn A, Stenmark H, Johansen T (2005) p62/SQSTMI forms protein aggregates degraded by autophagy and has a protective effect on huntingtin-induced cell death. J Cell Biol 171: 603-14
Bright N A, Davis L J, Luzio J P (2016) Endolysosomes Are the Principal Intracellular Sites of Acid Hydrolase Activity. Curr Biol 26:2233-45
Carlsson S R, Simonsen A (2015) Membrane dynamics in autophagosome biogenesis. Journal of cell science 128:193-205
Chang C, Young L N, Morris K L, von Bulow S, Schoneberg J, Yamamoto-Imoto H, Oe Y, Yamamoto K, Nakamura S, Stjepanovic G, Hummer G, Yoshimori T, Hurley J H (2019) Bidirectional Control of Autophagy by BECN1 BARA Domain Dynamics. Mol Cell 73: 339-353 e6
Cheng X T, Xie Y X, Zhou B, Huang N, Farfel-Becker T, Sheng Z H (2018) Characterization of LAMP1-labeled nondegradative lysosomal and endocytic compartments in neurons. J Cell Biol 217: 3127-3139
Diao J, Liu R, Rong Y, Zhao M, Zhang J, Lai Y, Zhou Q, Wilz L M, Li J, Vivona S, Pfuetzner R A, Branger A T, Zheng Q (2015) ATG14 promotes membrane tethering and fusion of autophagosomes to endolysosomes. Nature 520: 563-6
Dooley H C, Razi M, Poison H E, Girardin S E, Wilson M I, Tooze S A (2014) WIPI2 links LC3 conjugation with PDP, autophagosome formation, and pathogen clearance by recruiting Atg12-5-16L1. Mol Cell 55: 238-52
Engedal N, Seglen P O (2016) Autophagy of cytoplasmic bulk cargo does not require LC3. Autophagy 12:439-41
Eskelinen E L (2008) Fine structure of the autophagosome. Methods Mol Biol 445: 11-28
Florey O, Gammoh N, Kim S E, Jiang X, Overholtzer M (2015) V-ATPase and osmotic imbalances activate endolysosomal LC3 lipidation. Autophagy 11: 88-99
Fujita N, Morita E, Itoh T, Tanaka A, Nakaoka M, Osada Y, Umemoto T, Saitoh T, Nakatogawa H, Kobayashi S, Haraguchi T, Guan J L, Iwai K, Tokunaga F, Saito K, Ishibashi K, Akira S, Fukuda M, Noda T, Yoshimori T (2013) Recruitment of the autophagic machinery to endosomes during infection is mediated by ubiquitin. J Cell Biol 203: 115-28
Gammoh N, Florey O, Overholtzer M, Jiang X (2013) Interaction between FIP200 and ATG16L1 distinguishes ULK1 complex-dependent and -independent autophagy. Nat Struct Mol Biol 20: 144-9
Ganley I G, Espinosa E, Pfeffer S R (2008) A syntaxin 10-SNARE complex distinguishes two distinct transport routes from endosomes to the trans-Golgi in human cells. J Cell Biol 180: 159-72
Ganley I G, Lam du H, Wang J, Ding X, Chen S, Jiang X (2009) ULK1.ATG13.FIP200 complex mediates mTOR signaling and is essential for autophagy. J Biol Chem 284: 12297-305
Gao J, Reggiori F, Uugermann C (2018) A novel in vitro assay reveals SNARE topology and the role of Ykt6 in autophagosome fusion with vacuoles. J Cell Biol
Garcia D, Shaw R J (2017) AMPK: Mechanisms of Cellular Energy Sensing and Restoration of Metabolic Balance. Mol Cell 66: 789-800
Gatica D, Lahiri V, Klionsky D J (2018) Cargo recognition and degradation by selective autophagy. Nature, cell biology 20: 233-242
Gingras A C, Gygi S P, Raught B, Polaldewicz R D, Abraham R T, Hoekstra M F, Aebersold R, Sonenberg N (1999) Regulation of 4E-BP1 phosphorylation: a novel two-step mechanism. Genes Dev 13:1422-37
Guo B, Liang Q, Li L, Hu Z, Wu F, Zhang P, Ma Y, Zhao B, Kovacs A L, Zhang Z, Feng D, Chen S, Zhang H (2014) O-GlcNAc-modification of SNAP-29 regulates autophagosome maturation. Nature cell biology 16:1215-26
Guo H, Chitiprolu M, Roncevic L, Javalet C, Hemming F J, Thing M T, Meng L, Latreille E, Tanese de Souza C, McCulloch D, Baldwin R M, Auer R, Cote J, Russell R C, Sadoul R, Gibbings D (2017) Atg5 Disassociates the V1V0-ATPase to Promote Exosome Production and Tumor Metastasis Independent of Canonical Macroautophagy. Dev Cell 43: 716-730 e7
Gutierrez M G, Master S S, Singh S B, Taylor G A, Colombo M I, Deretic V (2004) Autophagy is a defense mechanism inhibiting BCG and Mycobacterium tuberculosis survival in infected macrophages. Cell 119: 753-66
Hamasaki M, Furata N, Matsuda A, Nezu A, Yamamoto A, Fujita N, Oomori H, Noda T, Haraguchi T, Hiraoka Y, Ainauo A, Yoshimori T (2013) Autophagosomes form at ER-mitochondria contact sites. Nature 495:389-93
Hatsuzawa K, Hirose H, Tani K, Yamamoto A, Scheller R H, Tagaya M (2000) Syntaxin 18, a SNAP Receptor That Functions in the Endoplasmic Reticulum, Intermediate Compartment, and cis-Golgi Vesicle Trafficking, Journal of Biological Chemistry 275:13713-13720
He C, Levine B (2010) The Beclin 1 interactome. Current opinion in cell biology 22: 140-9
Hong W, Lev S (2014) Tethering the assembly of SNARE complexes. Trends in cell biology 24: 35-43
Hosokawa N, Hara T, Kaizuka T, Kishi C, Takamura A, Miura Y, lemma S, Natsume T, Takehana K, Yamada N, Guan J L, Oshiro N, Mizushima N (2009) Nutrient-dependent mTORC1 association with the ULK1-Atg13-FIP200 complex required for autophagy. Molecular biology of the cell 20: 1981-91
Itakura E, Kishi-Itakura C, Mizushima N (2012) The hairpin-type tail-anchored SNARE syntaxin 17 targets to autophagosomes for fusion with endosomes/lysosomes. Cell 151:1256-69
Jahn R, Scheller R H (2006) SNAREs-engines for membrane fusion. Nature reviews Molecular cell biology 7: 631-43
Jao C C, Ragusa M J, Stanley R E, Hurley J H (2013) A HORMA domain in Atg13 mediates PI 3-kinase recruitment in autophagy. Proc Natl Acad Sci USA 110: 5486-91
Jung C H, Jun C B, Ro S H, Kim Y M, Otto N M, Cao J, Kundu M, Kim D H (2009) ULK-Atg13-FIP200 complexes mediate mTOR signaling to the autophagy machinery. Molecular biology of the cell 20:1992-2003
Kabeya Y, Mizushima N, Ueno T, Yamamoto A, Kirisako T, Noda T, Kominami E, Ohsumi Y, Yoshimori T (2000) LC3, a mammalian homologue of yeast Apg8p, is localized in autophagosome membranes after processing. Embo J 19: 5720-8
Katayama H, Kogure T, Mizushima N, Yoshimori T, Miyawaki A (2011) A sensitive and quantitative technique for detecting autophagic events based on lysosomal delivery. Chemistry & biology 18: 1042-52
Keown J R, Black M M, Ferron A, Yap M, Barnett M J, Pearce F G, Stoye J P, Goldstone D C (2018) A helical LC3-interacting region mediates the interaction between the retroviral restriction factor Trim5alpha and mammalian autophagy-related ATG8 proteins. J Biol Chem 293:18378-18386
Kim J, Kim Y C, Fang C, Russell R C, Kim J H, Fan W, Liu R, Zheng Q, Guan K L (2013) Differential regulation of distinct Vps34 complexes by AMPK in nutrient stress and autophagy. Cell 152: 290-303
Kim J, Kundu M, Viollet B, Guan K L (2011) AMPK and mTOR regulate autophagy through direct phosphorylation of Ulk1. Nature cell biology 13: 132-41
Kimura T, Jia J, Kumar S, Choi S W, Gu Y, Mudd M, Dupont N, Jiang S, Peters R, Farzam F, Jain A, Lidke K A, Adams C M, Johansen T, Deretic V (2017) Dedicated SNAREs and specialized TRIM cargo receptors mediate secretory autophagy. EMBO J 36: 42-60
Kimura T, Mandell M, Deretic V (2016) Precision autophagy directed by receptor regulators—emerging examples within the TRIM family. Journal of cell science 129: 881-91
Kirisako T, Baba M, Ishihara N, Miyazawa K, Ohsumi M, Yoshimori T, Noda T, Ohsumi Y (1999) Formation process of autophagosome is traced with Apg8/Aut7p in yeast J Cell Biol 147:435-46
Klionsky D J, Abdelmohsen K, Abe A, Abedin M J, Abeliovich H, Acevedo Arozena A, Adachi H, Adams C M. Adams P D, Adeli K, Adhihetty P J, Adler S G, Agam G, Agarwal R, Aghi M K, Agnello M. Agostinis P, Aguilar P V, Aguirre-Ghiso J, Airoldi E M et al. (2016) Guidelines for the use and interpretation of assays for monitoring autophagy (3rd edition). Autophagy 12:1-222
Klionsky D J, Abeliovich H, Agostinis P, Agrawal D K, Aliev G, Askew D S, Baba M, Baehrecke E H, Bahr B A, Ballabio A, Bamber B A, Bassham D C, Bergamini E, Bi X, Biard-Piechaczyk M, Blum J S, Bredesen D E, Brodsky J L, Brumell J H, Brunk U T et al. (2008) Guidelines for the use and interpretation of assays for monitoring autophagy in higher eukaryotes. Autophagy 4:151-75
Kriegenburg F, Ungermann C, Reggiori F (2018) Coordination of Autophagosome-Lysosome Fusion by Atg8 Family Members. Curr Biol 28: R512-R518
Kumar S, al. e (2019) Phosphorylation of Syntaxin 17 by TBK1 controls autophagy initiation Developmental Cell In press
Kumar S, Jain A, Farzam F, Jia J, Gu Y, Choi S W, Mudd M H, Claude-Taupin A, Wester M J, Lidke K A, Rusten T E, Deretic V (2018) Mechanism of Stx17 recruitment to autophagosomes via IRGM and mammalian Atg8 proteins. J Cell Biol 217:997-1013
Lawrence R E, Zoncu R (2019) The lysosome as a cellular centre for signalling, metabolism and quality control. Nature cell biology
Lazarou M, Sliter D A, Kane L A, Sarraf S A, Wang C, Burman J L, Sideris D P, Fogel A I, Youle R J (2015) The ubiquitin kinase PINK1 recruits autophagy receptors to induce mitophagy. Nature 524: 309-14
Levine B, Kroemer G (2019) Biological Functions of Autophagy Genes: A Disease Perspective. Cell 176:11-42
Lippincott-Schwartz J, Fambrough D M (1987) Cycling of the integral membrane glycoprotein, LEP100, between plasma membrane and lysosomes: kinetic and morphological analysis. Cell 49: 669-77
Liu L, Feng D, Chen G, Chen M, Zheng Q, Song P, Ma Q, Zhu C, Wang R, Qi W, Huang L, Xue P, Li B, Wang X, Jin H, Wang J, Yang F, Liu P, 22m Y, Sui S et al. (2012) Mitochondrial outer-membrane protein FUNDC1 mediates hypoxia-induced mitophagy in mammalian cells. Nature cell biology 14:177-85
Low S H, Chapin S J, Weimbs T, Komuves L G, Barnett M K, Mostov K E (1996) Differential localization of syntaxin isoforms in polarized Madin-Darby canine kidney cells. Molecular biology of the cell 7:2007-18
Luzio J P, Rous B A, Bright N A, Pryor P R, Mullock B M, Piper R C (2000) Lysosome-endosome fusion and lysosome biogoiesis. Journal of cell science 113:1515-24
Mallard F, Tang B L, Galli T, Tenza D, Saint-Pol A Yue X, Antony C, Hong W, Good B, Johannes L (2002) Early/recycling endosomes-to-TGN transport involves two SNARE complexes and a Rab6 isoform. J Cell Biol 156: 653-64
Malsam J, Söllner TH (2011) Organization of SNAREs within the Golgi Stack. Cold Spring Harbor Perspectives in Biology
Mandell M A, Jain A Arko-Mensah J, Chauhan S, Kimura T, Dinkins C, Silvestri G, Munch J, Kirchhoff F, Simonsen A Wei Y, Levine B, Johansen T, Deretic V (2014) TRIM proteins regulate autophagy and can target autophagic substrates by direct recognition. Dev Cell 30: 394-409
Manjithaya R, Nazarko T Y, Farre J C, Subramani S (2010) Molecular mechanism and physiological role of pexophagy. FEBS letters 584: 1367-73
Marino G, Pietrocola F, Eisenberg T, Kong Y, Malik S A Andryushkova A Schroeder S, Peudl T, Harger A Niso-Santano M, Zamzami N, Scoazec M, Durand S, Enot D P, Fernandez A F, Martins L Kepp O, Senovilla L, Bauvy C, Morselli E et al. (2014) Regulation of autophagy by cytosolic acetyl-coenzyme A. Mol Cell 53: 710-25
Matsui T, Jiang P, Nakano S, Sakamaki Y, Yamamoto H, Mizushima N (2018) Autophagosomal YKT6 is required for fusion with lysosomes independently of syntaxin 17. J Cell Biol 217: 2633-2645
McEwan D G, Popovic D, Gubas A Terawaki S, Suzuki H, Stadel D, Coxon F P, Miranda de Stegmann D, Bhogaraju S, Maddi K, Kirchof A, Gatti E, Helfrich M H, Wakatsuki S, Behrends C, Pierre P, Dikic I (2015) PLEKHM1 regulates autophagosome-lysosome fusion through HOPS complex and LC3/GABARAP proteins. Mol Cell 57: 39-54
Mizushima N, Noda T, Yoshimori T, Tanaka Y, Ishii T, George M D, Klionsky D J, Ohsumi M, Ohsumi Y (1998a) A protein conjugation system essential for autophagy. Nature 395: 395-8
Mizushima N, Sugita H, Yoshimori T, Ohsumi Y (1998b) A new protein conjugation system in human. The counterpart of the yeast Apg12p conjugation system essential for autophagy. J Biol Chem 273:33889-92
Mizushima N, Yoshimori T, Ohsumi Y (2011) The role of Atg proteins in autophagosome formation. Anna Rev Cell Dev Biol 27:107-32
Moreau K, Retina M, Rubinsztein D C (2013) Connections between SNAREs and autophagy. Trends Biochem Sci 38: 57-63
Muller J M, Shorter J, Newman R, Deinhardt K, Sagiv Y, Elazar Z, Warren G, Shima D T (2002) Sequential SNARE disassembly and GATE-16-GOS-28 complex assembly mediated by distinct NSF activities drives Golgi membrane fusion. J Cell Biol 157:1161-73
Nair U, Jotwani A, Geng J, Gammoh N, Richerson D, Yen W E, Griffith J, Nag S, Wang K, Moss T, Baba M, McNew J A, Jiang X, Reggiori F, Melia T J, Klionsky D J (2011) SNARE proteins are required for macroautophagy. Cell 146: 290-302
Nakamura S, Yoshimori T (2017) New insights into autophagosome-lysosome fusion. Journal of cell science 130:1209-1216
Nakatogawa H, Ichimura Y, Ohsumi Y (2007) Atg8, a ubiquitin-like protein required for autophagosome formation, mediates membrane tethering and hemifusion. Cell 130: 165-78
Nguyen T N, Padman B S, Lazarou M (2016a) Deciphering the Molecular Signals of PINK1/Parkin Mitophagy. Trends in cell biology 26:733-744
Nguyen T N, Padman B S, Usher J, Oorschot V, Ramm G, Lazarou M (2016b) Atg8 family LC3/GABARAP proteins are crucial for autophagosome-lysosome fusion but not autophagosome formation during PINK1/Parkin mitophagy and starvation. J Cell Biol 215: 857-874
Nishimura T, Kaizuka T, Cadwell K, Sahani M H, Saitoh T, Akira S, Virgin H W, Mizushima N (2013) FIP200 regulates targeting of Atg16L1 to the isolation membrane. EMBO Rep 14: 284-91
Noda T, Ohsumi Y (1998) Tor, a phosphatidylinositol kinase homologue, controls autophagy in yeast. J Biol Chem 273: 3963-6
Olsvik H L, Lamark T, Takagi K, Bowitz Larsen K, Evjen G, Overvatn A, Mizushima T, Johansen T (2015) FYCO1 Contains a C-terminally Extended, LC3A/B-preferring LC3-Interacting Region (LIR) Motif Required for Efficient Maturation of Autophagosomes During Basal Autophagy. J Biol Chem
Park J M, Jung C H, Seo M, Otto N M, Grunwald D, Kim K H, Moriarity B, Kim Y M, Starker C, Nho R S, Voytas D, Kim D H (2016) The ULK1 complex mediates MTORC1 signaling to the autophagy initiation machinery via binding and phosphorylating ATG 14. Autophagy 12: 547-64
Petiot A, Ogier-Denis E, Blommaart E F, Meijer A J, Codogno P (2000) Distinct classes of phosphatidylinositol 3′-kinases are involved in signaling pathways that control macroautophagy in HT-29 cells. J Biol Chan 275:992-8.
Pols M S, van Meel E, Oorschot V, ten Brink C, Fukuda M, Swetha M G, Mayor S, Klumperman J (2013) hVps41 and VAMP7 function in direct TGN to late endosome transport of lysosomal membrane proteins. Nat Commun 4:1361
Ponpuak M, Mandell M A, Kimura T, Chanhan S, Cleyrat C, Deretic V (2015) Secretory autophagy. Curr Opin Cell Biol 35: 106-116
Popelka H, Klionsky D J (2015) Analysis of the native conformation of the LIR/AIM motif in the Atg8/LC3/GABARAP-binding proteins. Autophagy 11:2153-9
Randow F, Youle R J (2014) Self and nonself: how autophagy targets mitochondria and bacteria. Cell Host Microbe 15: 403-11
Rasmussen M S, Birgisdottir A B, Johansen T (2019) Use of Peptide Arrays for Identification and Characterization of LIR Motifs. Methods Mol Biol 1880: 149-161
Rogov V, Dotsch V, Johansen T, Kirkin V (2014) Interactions between autophagy receptors and ubiquitin-like proteins form the molecular basis for selective autophagy. Mol Cell 53: 167-78
Rogov W, Stolz A, Ravichandran A C, Rios-Szwed D O, Suzuki H, Kniss A, Lohr F, Wakatsuki S, Dotsch V, Dikic I, Dobson R C, McEwan D G (2017) Structural and functional analysis of the GABARAP interaction motif (GIM). EMBO Rep 18:1382-1396 Saftig P, Klumperman J (2009) Lysosome biogenesis and lysosomal membrane proteins: trafficking meets function. Nature reviews Molecular cell biology 10: 623-35
Sagiv Y, Legesse-Miller A, Porat A, Elazar Z (2000) GATE-16, a membrane transport modulator, interacts with NSF and the Golgi v-SNARE GOS-28. EMBO J 19: 1494-504
Sancak Y, Bar-Peled L, Zoncu R, Markhard A L, Nada S, Sabatini D M (2010) Ragulator-Rag complex targets mTORC1 to the lysosomal surface and is necessary for its activation by amino acids. Cell 141:290-303
Sandoval H, Thiagarajan P, Dasgupta S K, Schumacher A, Prchal J T, Chen M. Wang J (2008) Essential role for Nix in autophagic maturation of erythroid cells. Nature 454:232-5
Sanjana N E, Shalem O, Zhang F (2014) Improved vectors and genome-wide libraries for CRISPR screening. Nat Methods 11: 783-784
Sanjuan M A, Dillon C P, Tait S W, Moshiach S, Dorsey F, Connell S, Komatsu M, Tanaka K, Cleveland J L, Withoff S, Green D R (2007) Toll-like receptor signalling in macrophages links the autophagy pathway to phagocytosis. Nature 450:1253-7
Saxton R A, Sabatini D M (2017) mTOR Signaling in Growth, Metabolism, and Disease. Cell 168:960-976
Scott R C, Schuldiner O, Neufeld T P (2004) Role and regulation of starvation-induced autophagy in the Drosophila fat body. Dev Cell 7:167-78
Seglen P O, Luhr M, Mills I G, Saetre F, Szalai P, Engedal N (2015) Macroautophagic cargo sequestration assays. Methods 75:25-36
Shpilka T, Weidberg H, Pietrokovski S, Elazar Z (2011) Atg8: an autophagy-related ubiquitin-like protein family. Genome Biol 12: 226
Skytte Rasmussen M, Mouilleron S, Kumar Shrestha B, Wirth M, Lee R, Bowitz Larsen K, Abudu Princely Y, O'Reilly N, Sjottem E, Tooze S A, Lamark T, Johansen T (2017) ATG4B contains a C-terminal LIR motif important for binding and efficient cleavage of mammalian orthologs of yeast Atg8. Autophagy 13: 834-853
Stadel D, Millarte V, Tillmann K D, Huber J, Tamin-Yecheskel B C, Akutsu M, Demishtein A, Ben-Zeev B, Anikster Y, Perez F, Dotsch V, Elazar Z, Rogov V, Farhan H, Behrends C (2015) TECPR2 Cooperates with LC3C to Regulate COPII-Dependent ER Export. Mol Cell 60: 89-104
Stolz A, Ernst A, Dikic I (2014) Cargo recognition and trafficking in selective autophagy. Nature cell biology 16:495-501
Subramaniam V N, Peter F, Philp R, Wong S H, Hong W (1996) GS28, a 28-Kilodalton Golgi SNARE That Participates in ER-Golgi Transport. Science 272:1161-1163
Tai G, Lu L, Wang T L, Tang B L, Goud B, Johannes L, Hong W (2004) Participation of the syntaxin 5/Ykt6/GS28/GS 15 SNARE complex in transport from the early/recycling endosome to the trans-Golgi network. Molecular biology of the cell 15:4011-22
Takats S, Glatz G, Szenci G, Boda A, Horvath G V, Hegedus K, Kovacs A L, Juhasz G (2018) Non-canonical role of the SNARE protein Ykt6 in autophagosome-lysosome fusion. PLoS Genet 14: e1007359
Takats S, Nagy P, Varga A, Pircs K, Karpati M, Varga K, Kovacs A L, Hegedus K, Juhasz G (2013) Autophagosomal Syntaxin 17-dependent lysosomal degradation maintains neuronal function in Drosophila. J Cell Biol 201:531-9
Tan X Liao Z, Liang H, Chen X Zhang B, Chu L (2018) Upregulation of liver kinase B1 predicts poor prognosis in hepatocellular carcinoma. International journal of oncology 53: 1913-1926
Tanaka Y, Guhde G, Suter A, Eskelinen E L, Hartmann D, Lullmann-Rauch R, Janssen P M Blanz J, von Figura K, Saftig P (2000) Accumulation of autophagic vacuoles and cardiomyopathy in LAMP-2-deficient mice. Nature 406: 902-6
Tsuboyama K, Koyama-Honda I, Sakamaki Y, Koike M Morishita H, Mizushima N (2016) The ATG conjugation systems are important for degradation of the inner autophagosomal membrane. Science 354:1036-1041
Velikkakath A K, Nishimura T, Oita E, Ishihara N, Mizushima N (2012) Mammalian Atg2 proteins are essential for autophagosome formation and important for regulation of size and distribution of lipid droplets. Molecular biology of the cell 23: 896-909
Violot S, Carpentier P, Blanchoin L, Bourgeois D (2009) Reverse pH-dependence of chromophore protonation explains the large Stokes shift of the red fluorescent protein mKeima. J Am Chem Soc 131:10356-7
von Muhlinen N, Akutsu M Ravenhill B J, Foeglein A, Bloor S, Rutherford T J, Freund S M, Komander D, Randow F (2012) LC3C, Bound Selectively by a Noncanonical LIR Motif in NDP52, Is Required for Antibacterial Autophagy. Molecular cell 48: 329-42
Wang Y, Foo L Y, Guo K, Gan B Q, Zeng Q, Hong W, Tang B L (2006) Syntaxin 9 is Enriched in Skin Hair Follicle Epithelium and Interacts With the Epidermal Growth Factor Receptor. Traffic 7: 216-226
Wang Z, Miao G, Xue X, Guo X, Yuan C, Wang Z, Zhang G, Chen Y, Feng D, Hu J, Zhang H (2016) The Vici Syndrome Protein EPG5 Is a Rab7 Effector that Determines the Fusion Specificity of Autophagosomes with Late Endosomes/Lysosomes. Mol Cell 63: 781-95
Wei Y, Chiang W C, Sumpter R, Jr., Mishra P, Levine B (2017) Prohibitin 2 Is an Inner Mitochondrial Membrane Mitophagy Receptor. Cell 168:224-238 e10
Weidberg H, Shpilka T, Shvets E, Abada A Shimron F, Elazar Z (2011) LC3 and GATE-16 N termini mediate membrane fusion processes required for autophagosome biogenesis. Developmental cell 20: 444-54
Weidberg H, Shvets E, Shpilka T, Shimron F, Shinier V, Elazar Z (2010) LC3 and GATE-16/GABARAP subfamilies are both essential yet act differently in autophagosome biogenesis. The EMBO journal 29:1792-802
Wild P, Farhan H, McEwan D G, Wagner S, Rogov W, Brady N R, Richter B, Korac J, Waidmann O, Choudhary C, Dotsch V, Bumann D, Dikic I (2011) Phosphorylation of the autophagy receptor optineurin restricts Salmonella growth. Science 333:228-33
Wubbolts R, Fernandez-Borja M, Oomen L, Verwoerd D, Janssen H, Calafat J, Tulp A, Dusseljee S, Neefjes J (1996) Direct vesicular transport of MHC class II molecules from lysosomal structures to the cell surface. J Cell Biol 135:611-22
Xie Z, Nair U, Klionsky D J (2008) Atg8 controls phagophore expansion daring autophagosome formation. Molecular biology of the cell 19:3290-8
Youle R J, Narendra D P (2011) Mechanisms of mitophagy. Nature reviews Molecular cell biology 12: 9-14
Young A R, Chan E Y, Hu X W, Kochi R, Crawshaw S G, High S, Hailey D W, Lippincott-Schwartz J, Tooze S A (2006) Starvation and ULK1-dependent cycling of mammalian Atg9 between the TGN and endosomes. Journal of cell science 119: 3888-900
Zhang J, Tripathi D N, Jing J, Alexander A, Kim J, Powell R T, Dere R, Tait-Mulder J, Lee J H, Paull T T, Pandita R K, Charaka V K, Pandita T K, Kastan M B, Walker C L (2015) ATM functions at the peroxisome to induce pexophagy in response to ROS. Nature cell biology 17: 1259-1269.

1. A method of treating an autophagy-mediated disease state or condition in a patient or subject in need comprising administering an ATg8 modulator and optionally a STX16 and/or STX17 modulator all in therapeutically effective amounts to said subject.

2. The method according to claim 1 wherein said ATg8 modulator is an ATg8 inhibitor.

3. The method according to claim 1 wherein said ATg8 modulator is an ATg8 agonist.

4. The method according to claim 2 wherein said ATg8 inhibitor is combined with a therapeutically effective amount of a STX16 and/or a STX17 inhibitor.

5. The method according to claim 4 wherein said treatment provides a synergistic effect on the autophagy-mediated disease state or condition.

6. The method according to claim 2 wherein said disease state or condition is cancer or an autoimmune disease.

7. The method according to claim 2 wherein said disease state or condition is rheumatoid arthritis, malaria, antiphospholipid antibody syndrome, lupus, chronic urticarial, Sjogren's disease, autoimmune-related Type 1 diabetes, rheumatoid arthritis (RA), psoriasis/psoriatic arthritis, multiple sclerosis, inflammatory bowel disease (IBD) including Crohn's disease and ulcerative colitis, Addison's disease, Grave's disease, Hashimoto's thyroiditis, Myasthenia gravis, autoimmune vasculitis, pernicious anemia and celiac disease.

8. (canceled)

9. The method according to claim 6 wherein said disease state or condition is an autoimmune disease.

10. The method according to claim 2 wherein said ATg8 inhibitor is a TBK1 antagonist, an anti-ATg8 antibody or a compound according to the chemical structure: 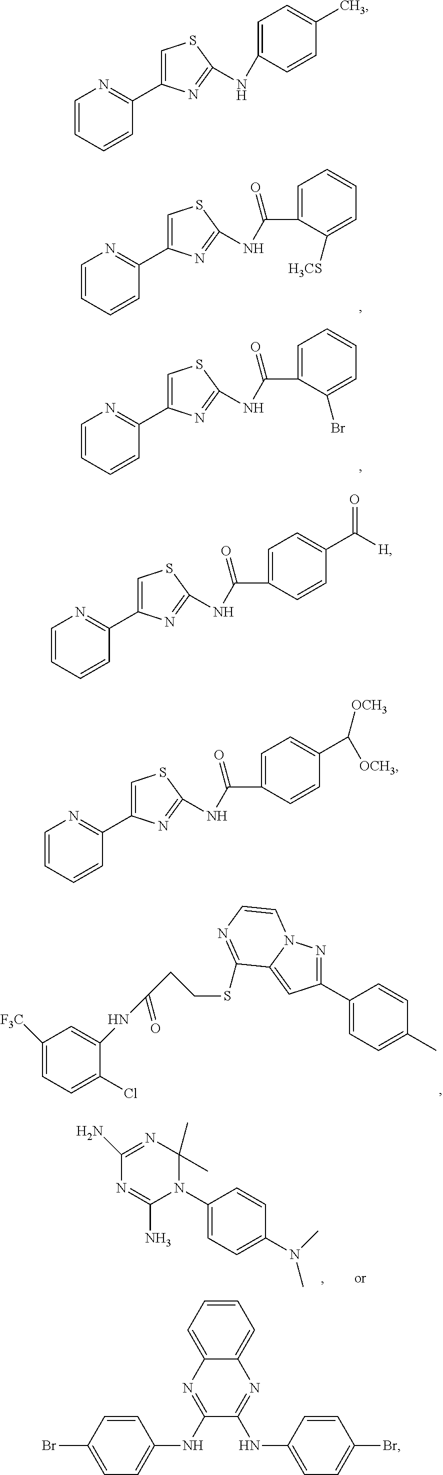
Or a pharmaceutically acceptable salt thereof or a mixture thereof.

11-13. (canceled)

14. The method according to claim 4 wherein said STX16 inhibitor is an anti-STX16 antibody or a small interfering RNA (SiRNA) having one of the following sequences:
(SEQ ID NO: 1)
GAACAUGCCAUUGAGAUAA;
(SEQ ID NO: 2)
AACCGACGCUUUCUUGUUG;
(SEQ ID NO: 3)
GGUGUCAGGCAUCAGCUUA
or
(SEQ ID NO: 4)
GUAUGAUGUUGGCCGGAUU.

15. The method according to claim 4 wherein said STX17 inhibitor is an anti-STX antibody, the compound AG1478 (Tyrphostin AG1478) or AG1024 (Tyrphostin AG1024) or a TPK1 inhibitor according to any of claims 11-13 hereof.

16. The method according to claim 2 wherein said ATg8 agonist is combined with a therapeutically effective amount of a STX16 and/or a STX17 agonist.

17. (canceled)

18. (canceled)

19. The method according to claim 16 wherein said treatment provides a synergistic effect on the autophagy-mediated disease state or condition.

20. (canceled)

21. The method according to claim 1 wherein said autophagy-mediated disease state or condition is hepatic encephalopathy, liver toxicity from heavy metals, especially cadmium, Alzheimer's disease, Parkinson's disease, mild cognitive impairment, autism, mitochondrial lymphoblast dysfunction, mitochondrial fibroblast dysfunction, mitochondrial neuronal dysfunction, mitochondrial cardiac dysfunction, cardiac hypertrophy, mitochondrial adrenocortical dysfunction, Zellweger syndrome, rhizomelic chondrodysplasia pimelate, infantile refsum disease, Alzheimer's disease, gastric cancer, Diamond-Blackfan anemia, Treacher-Collins Syndrome (TCS), Native American Indian childhood Cirrhosis (NAICC), male infertility, Bowen-Conradi syndrome (BCS), alopecia-neurological defects-endocrinopathy syndrome (AWE syndrome), Schwachman-Diamond Syndrome, primary open angle glaucoma (POAG), neurofibromatosis type 1 (NFI), sever macrocytic anemia, myelodysplastic syndrome, predisposition to cancer and Mycoplasma infections.

22. The method according to claim 19 wherein said Mycoplasma infection is a M. tuberculosis infection.

23. A method of treating an autophagy-mediated disease state or condition in a patient or subject in need comprising co-administering a STX16 and STX17 modulator, each in therapeutically effective amounts to said subject, optionally in combination with an ATg8 modulator, said method producing a synergistic therapeutic effect in said patient or subject.

24. The method according to claim 23 wherein said STX16 and STX17 modulators are inhibitors of STX16 and STX17.

25. The method according to claim 23 wherein said STX16 and STX17 modulators are agonists of STX16 and STX17.

26. The method according to claim 24 wherein an ATg8 inhibitor is combined with said STX16 and said STX17 inhibitors.

27. The method according to claim 25 wherein an ATg8 agonist is combined with said STX16 and said STX17 agonists.

28. The method according to claim 23 wherein said disease state or condition is cancer or an autoimmune disease.

29. The method according to claim 23 wherein said disease state or condition is rheumatoid arthritis, malaria, antiphospholipid antibody syndrome, lupus, chronic urticarial, Sjogren's disease, autoimmune-related Type 1 diabetes, rheumatoid arthritis (RA), psoriasis/psoriatic arthritis, multiple sclerosis, inflammatory bowel disease (IBD) including Crohn's disease and ulcerative colitis, Addison's disease, Grave's disease, Hashimoto's thyroiditis, Myasthenia gravis, autoimmune vasculitis, pernicious anemia and celiac disease.

30. (canceled)

31. (canceled)

32. The method according to claim 23 wherein said STX17 inhibitor is a TBK1 antagonist, an anti-STX17 antibody or the compound AG1478 (Tyrphostin AG1478) or AG1024 (Tyrphostin AG1024).

33. (canceled)

34. (canceled)

35. (canceled)

36. The method according to claim 26 wherein said Atg8 inhibitor is a TK1 antagonist, an anti-ATg8 antibody or a compound according to the chemical structure: 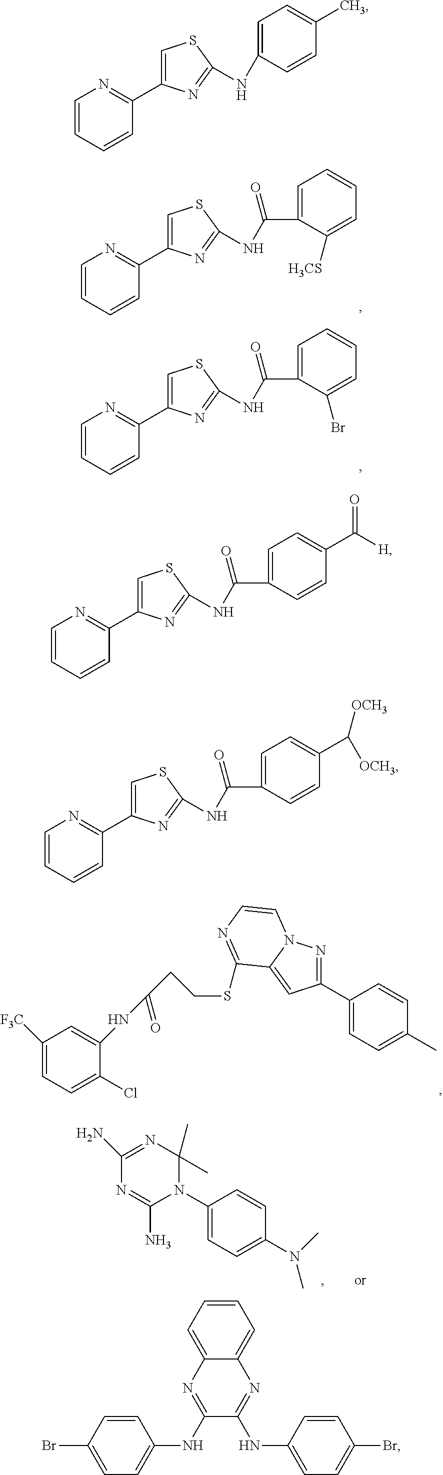
Or a pharmaceutically acceptable salt thereof or a mixture thereof.

37. The method according to claim 23 wherein said STX16 inhibitor is an anti-STX16 antibody or a small interfering RNA (SiRNA) having one of the following sequences:
(SEQ ID NO: 1)
GAACAUGCCAUUGAGAUAA;
(SEQ ID NO: 2)
AACCGACGCUUUCUUGUUG;
(SEQ ID NO: 3)
GGUGUCAGGCAUCAGCUUA
or
(SEQ ID NO: 4)
GUAUGAUGUUGGCCGGAUU.

38. (canceled)

39. The method according to claim 27 wherein said STX16 agonist is metformin, phenformin, buformin, proguanil, chlorproguanil, bromhexine, ambroxol, 20-hydroxyecdysone, a copper salt, a cobalt salt or a mixture thereof.

40. The method according to claim 27 wherein said STX17 agonist is Dimethhylxanthenone-4-Acetic acid (XAA-5Me); 5-Me5thyl-xanthenone-4-Acetic Acid; c-diGMP (cyclic di-GMP) or an Interferon Type I selected from the group consisting of IFN-α, IFN-β, IFN-ε IFN-κ, IFN-δ, IFN-τ, IFN-ω, IFN-ν or a mixture thereof.

41. (canceled)

42. The method according to claim 23 wherein said autophagy-mediated disease state or condition is hepatic encephalopathy, liver toxicity from heavy metals, especially cadmium, Alzheimer's disease, Parkinson's disease, mild cognitive impairment, autism, mitochondrial lymphoblast dysfunction, mitochondrial fibroblast dysfunction, mitochondrial neuronal dysfunction, mitochondrial cardiac dysfunction, cardiac hypertrophy, mitochondrial adrenocortical dysfunction, Zellweger syndrome, rhizomelic chondrodysplasia punctate, infantile refsum disease, Alzheimer's disease, gastric cancer, Diamond-Blackfan anemia, Treacher-Collins Syndrome (TCS), Native American Indian childhood Cirrhosis (NAICC), male infertility, Bowen-Conradi syndrome (BCS), alopecia-neurological defects-endocrinopathy syndrome (AWE syndrome), Schwachman-Diamond Syndrome, primary open angle glaucoma (POAG), neurofibromatosis type 1 (NFI), sever macrocytic anemia, myelodysplastic syndrome, predisposition to cancer and Mycoplasma infections.

43. The method according to claim 42 wherein said Mycoplasma infection is a M. tuberculosis infection.

44. A method of treating coronavirus in a patient in need, said method comprising administering to said patient a therapeutically effective amount of a STX17 agonist.

45. The method according to claim 44 wherein said STX17 agonist is Dimethhylxanthenone-4-Acetic acid (XAA-5Me); 5-Me5thyl-xanthenone-4-Acetic Acid; c-diGMP (cyclic di-GMP) or an Interferon Type I selected from the group consisting of IFN-α, IFN-β, IFN-ε IFN-κ, IFN-δ, IFN-τ, IFN-ω, IFN-ν or a mixture thereof.

46. The method according to claim 44 wherein said STX17 agonist is combined with a therapeutically effective amount of an ATg8 agonist or a STX16 agonist.

47. (canceled)

48. A pharmaceutical composition comprising an effective amount of a STX16 modulator and a STX17 modulator in further combination with a pharmaceutically acceptable carrier, additive or excipient.

49. The composition according to claim 49 further including a ATg8 modulator.

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)
